(19) United States
(12) Patent Application Publication
(10) Pub. No.: US 2018/0179552 A1
(43) Pub. Date: Jun. 28, 2018

(54) Methods for Inducing Apomixis in Plants

(71) Applicant: LEIBNIZ-INSTITUT FÜR PFLANZENGENETIK UND KULTURPFLANZENFORSCHUNG (IPK), Gatersleben (DE)

(72) Inventors: Timothy Sharbel, Saskatoon, Saskatchewan (CA); José M. Corral, Quedlinburg (DE)

(21) Appl. No.: 15/814,618

(22) Filed: Nov. 16, 2017

Related U.S. Application Data
(62) Division of application No. 14/648,401, filed on May 29, 2015, now abandoned.

(30) Foreign Application Priority Data
Nov. 29, 2012 (EP) 12194821.0

(51) Int. Cl. C12N 15/82; C12N 9/22

(52) U.S. Cl. CPC C12N 15/8287; C12N 15/8217; C12N 9/22

(57) ABSTRACT

The present invention relates to methods for inducing apomixis in a plant, methods for the production of apomictic plants and the plants and plant seeds obtained thereby.

### CROSS-REFERENCE TO RELATED APPLICATIONS

This application is a Division of U.S. National Phase application Ser. No. 14/648,401, filed May 29, 2015, which is a 35 U.S.C. § 371 National Phase of International Patent Application No. PCT/EP2013/074842, filed Nov. 27, 2013 and incorporated herein by reference in its entirety, which claims the benefit of European Patent Application No. 12194821.0, filed Nov. 29, 2012, which is incorporated herein by reference in its entirety.

### INCORPORATION OF SEQUENCE LISTING

A computer readable form of the sequence listing named “WO_2014083047.txt” and which is 448727 bytes in size and created on May 29, 2015, is electronically filed herewith and is incorporated herein by reference in its entirety. This Sequence Listing consists of SEQ ID NOs: 1-119.

### DESCRIPTION

The present invention relates to methods for inducing apomixis in a plant, methods for the production of apomictic plants and the plants and plant seeds obtained thereby.

Apomixis in flowering plants is defined as the asexual formation of a seed from the maternal tissues of the ovule, avoiding the processes of meiosis and fertilisation, leading to embryo development (Bicknell and Koltunow, 2004). As a consequence, plants generated from apomictically formed seeds are genetically identical to their progenitor.

Generally speaking, apomixis is characterised by the production without meiosis of an unreduced egg cell (apomeiosis) which undergoes parthenogenetic development into an embryo which is genetically identical to the mother plant. Some aspects of sexuality can be maintained, as fertilisation (i. e. pseudogamy) is for the most part obligate for the production of a functional endosperm (i. e. the embryo's nourishing tissue) with a balanced maternal to paternal genome ratio.

Naturally occurring vegetative, non-sexual reproduction in plants through seeds, also called apomixis, is a genetically controlled reproductive mechanism of plants primarily found in some polyploid non-cultivated species. Various types of apomixis, inter alia gametophytic and sporophytic, can be distinguished. In sporophytic apomixis also called adventitive embryony, a somatic embryo develops not from the gametophyte but directly from the cells of the nucellus, ovary wall or integuments. Somatic embryos from surrounding cells invade the sexual ovary, one of the somatic embryos out-competes the other somatic embryos and the sexual embryo, and utilizes the produced endosperm.

Gametophytic apomixis is a naturally-occurring type of asexual seed formation whereby progeny, which are clonal to the maternal genotype, are produced from meiotically-unreduced embryo sacs, i. e. the female gametophyte. Most gametophytic apomictic species are found in the Asteraceae, Rosaceae and Poaceae, where they have arisen independently and recurrently. Polyploidy, facultative apomixis (both sexual and apomictic seed production within one individual), and faster development of the apomeiotic ovule relative to the sexual one are traits which are shared among most of these taxa.

Apomixis is derived from sex, and three independent developmental steps must be acquired for a sexual plant to produce seeds apomictically: the formation of an unreduced megaspore, that means the formation of an embryo sac having the same ploidy as the somatic cells of the mother plant from a meiotically-unreduced megaspore (diplospory, apomeiosis) or from nucellar cell (apospory), the subsequent development of an embryo from an unreduced egg in the absence of fertilization (parthenogenesis) and fertilization of the binucleate central cell to form a functional endosperm (pseudogamy). The term “apomeiosis” covers both apospory and diplospory. The apomeiotically-derived embryo thus receives its entire genome through the female line. As these components are under separate genetic control, it has been difficult to envision how all three could evolve in unison in a sexual ancestor considering random mutations, since the expression of any single step would decrease the fitness of its sexual carrier. It is widely accepted that apomictic seed development results from deregulation of the sexual development pathway, which would be manifested at multiple loci simultaneously. In wild apomictic taxa, this coordinated deregulation is hypothesized to be influenced by global regulatory changes resulting from hybridization and/or polyploidy (Grossniklaus, 2001, From sexuality to apomixis: Molecular and genetic approaches, In: The flowering of apomixis: From Mechanisms to Genetic Engineering, 168-211).

Recent reports analyse the gene expression of apomeiosis, that means unreduced gamete formation, in microdissected ovules of Boechera, and were able to identify quite a large number of differentially expressed alleles between sexual and apomeiotic ovules in a particular stage of the development, namely the megaspore mother cell (MMC) stage. Further studies focussed on heterochrony of gene expression patterns over a series of developmental stages in sexual and apomeiotic ovules (Sharbel et al., 2009, The Plant Journal, 58, 870-882, Sharbel et al., 2010, The Plant Cell, 22, 655-671). However, although the state of the art expectedly show that apomictic and sexual ovules are characterised by specific molecular signatures, it does not provide any clue on how to induce apomixis in a desired plant in a reliable and foreseeable manner, in particular by means of conventional gene transfer techniques.

In fact, one of the main difficulties in identifying the molecular genetic mechanisms controlling apomixis is that the genomes of virtually all apomicts are both polyploidy and hybrid in nature. Although considerable efforts, including in-depth functional molecular analyses, have been undertaken to analyse the molecular framework underlying apomictic phenomena, so far it still remains a challenge to control separately for the influences of either effect, both of which can have diverse regulatory consequences.

Engineering apomixis to a controllable, more reproducible trait would provide many advantages in plant improvement and cultivar development. Apomixis would provide for true-breeding, seed propagated hybrids. Harnessing apomixis would, thus, greatly facilitate and accelerate the ability of plant breeders to fix and faithfully propagate genetic heterozygosity and associated hybrid vigour in crop plants. Moreover, apomixis could shorten and simplify conventional breeding processes so that selfing and progeny testing to produce or stabilize a desirable gene combination could be eliminated.

The controlled use of apomixis would therefore certainly simplify commercial hybrid seed production. In particular, the need for physical isolation of commercial hybrid production fields would be eliminated, available land could be used to grow hybrid seed instead of dividing space between pollinators and male sterile lines and finally the need to maintain parental line seed stocks would be eliminated.

Apomixis would provide for the use as cultivars of genotypes with unique gene combinations since apomictic genotypes breed true irrespective of heterozygosity. Genes or groups of genes could thus be fixed in super genotypes. Every superior apomictic genotype from a sexual-apomictic cross would have the potential to be a cultivar. Apomixis would therefore allow plant breeders to develop cultivars with specific stable traits for such characters as height, seed and forage quality and maturity.

Thus, the application of apomixis in agriculture is considered an important enabling technology that would greatly facilitate the fixation and faithful propagation of genetic heterozygosity and associated hybrid vigor in crop plants (Spillane, 2004, Nat Biotech 22(6), 687-691).

All these potential benefits which rely on the production of seed via apomixis are presently, however, unrealized, to a large extent because of the problem of engineering apomictic capacity into plants of interest.

US 2002/0069433 A1 discloses methods for increasing the probability of vegetative reproduction of a new plant generation wherein a gene which encodes a protein acting in the signal transduction cascade triggered by the somatic embryogenesis receptor kinase is transgenically expressed. US 2008/0155712 A1 discloses processes for identifying in a plant, in particular maize, sequences responsible for apomictic development, in particular by genome mapping. WO 99/35258 A1 discloses nucleic acid markers for an apospory specific genomic region from the genus Pennisetum. U.S. Pat. No. 7,541,514 B2 discloses methods for producing apomictic plants from sexual plants by selecting, collecting and breeding specific plant lines.

None of said disclosures provide methods which can easily be used in gene transfer methods to obtain in a controllable and inexpensive way apomixis in plants.

The technical problem underlying the present invention is therefore to provide methods to overcome the above-identified problems, in particular to provide methods to introduce apomixis into a plant for instance by means of recombinant gene technology, in particular by means of recombinant DNA transfer technology, in particular to provide methods to induce apomixis in plants and to obtain apomictic plants, in particular in a controllable, foreseeable, reliable, easy and cost-effective way.

The present invention solves its underlying problem by the provision of the teaching of the independent claims, in particular by the provision of methods to induce apomixis in plants, methods to produce apomictic plants and plants obtained thereby.

Accordingly, the present invention relates to a method for the production of a transgenic apomictic plant, comprising the following steps:
a) providing a plant cell,
b) transforming said plant cell with at least one plant vector containing at least one exogenous nucleotide sequence element so as to obtain a transgenic plant cell comprising said at least one exogenous nucleotide sequence element and which transgenic plant cell comprises a nucleotide sequence coding for a trans-acting apomixis effector, a cis-acting regulatory element and a nucleotide sequence coding for a protein with the activity of a DEDDh exonuclease, which is under control of said cis-acting regulatory element, wherein said transacting apomixis effector is capable of interacting with said ci-sacting regulatory element and wherein said cis-acting regulatory element comprises at least one regulatory nucleotide core sequence selected from the group consisting of
the ATHB-5 binding site of any one of SEQ ID No. 66 or 67, the LIM-1 binding site of any one of SEQ ID No. 68 to 73, the SORLIP1AT binding site of any one of SEQ ID No. 74 or 75, the SORLIP2AT binding site of any one of SEQ ID No. 76 or 77 and the POLASIG1 binding site of any one of SEQ ID No. 78 or 79, and
c) regenerating the transformed plant cell into a transgenic plant exhibiting apomixis.

Thus, the present invention provides methods for the production of a transgenic apomictic plant. These methods comprise, in a preferred embodiment consist of, a series of process steps a), b) and c). Performing said process steps a), b) and c) is also suitable to induce apomixis in a plant. Thus, the present invention also relates to a method for inducing apomixis in a plant, in particular consisting of, comprising the above-identified steps a), b) and c). For such a teaching the following technical considerations of the present invention apply as well as is evident to the skilled person.

The present method teaches to provide a plant cell, in particular a plant cell from a sexually propagating plant, and to transform said plant cell with at least one plant vector containing at least one exogenous nucleotide sequence element so as to obtain a transgenic plant cell, that means a plant cell which comprises in addition to the genetic material being endogenously present in the plant cell provided in step a) at least one exogenous nucleotide sequence element which is thus naturally not present or not present at the specific genomic position in said plant cell provided in step a). Said at least one exogenous nucleotide sequence element which is transferred by the plant vector into the plant cell is a nucleotide sequence coding for a trans-acting apomixis effector, a cis-acting regulatory element, in particular a promoter, most preferably a promoter containing a regulatory nucleotide core sequence, or comprises both. In a preferred embodiment, said exogenous nucleotide sequence element comprises a cis-acting regulatory element functionally and operably linked to a nucleotide sequence coding for a protein with the activity of a DEDDh exonuclease. The transgenic plant being transformed with said vector thus receives stably integrated into its genome said at least one exogenous nucleotide sequence element.

Said transgenic plant cell being obtained in process step b) comprises a nucleotide sequence coding for a trans-acting apomixis effector, a cis-acting regulatory element and a nucleotide sequence coding for a protein with the activity a DEDDh exonuclease, wherein said nucleotide sequence coding for a protein with the activity of a DEDDh exonuclease is under regulatory control, in particular under transcriptional control, of said cis-acting regulatory element and wherein at least the nucleotide sequence coding for the trans-acting apomixis effector or the cis-acting regulatory element, optionally operably linked to a nucleotide sequence coding for a protein with the activity of a DEDDh exonuclease, has been transformed into said plant cell in process step b). Accordingly, at least one of these two above-identified nucleotide sequences is an exogenous nucleotide sequence being introduced into the plant cell to be transformed.

The trans-acting apomixis effector is in a particularly preferred embodiment a trans-acting transcription factor, in particular a DNA-binding transcription factor.

In a preferred embodiment of the present invention the plant vector used to transform the plant cell provided in step (a) comprises as an exogenous nucleotide sequence element a nucleotide sequence coding for said trans-acting apomixis effector or a nucleotide sequence comprising said cis-acting regulatory element or both. Thus, the present invention postulates that the exogenous nucleotide sequence element characterising the transgenic plant cell obtained in step b) by transforming a plant cell is either a nucleotide sequence coding for a trans-acting apomixis effector or a nucleotide sequence comprising a cis-acting regulatory element, in particular a so called “regulatory nucleotide core sequence” of the present invention or both. In a particularly preferred embodiment, said plant vector comprises—in case it contains the cis-acting regulatory element—further the nucleotide sequence for a protein with the activity of a DEDDh exonuclease operably linked to said cis-acting regulatory element. Thus, the presently obtained transgenic plant cell of process step b) is characterised by the presence of a transgenic nucleotide sequence comprising a trans-acting apomixis effector, a transgenic cis-acting regulatory element, in particular a regulatory nucleotide core sequence of the present invention, or both, and wherein said nucleotide sequences are either not endogenously present in the plant cell provided in step a) or not present at said specific genomic location achieved after the transformation step.

The present teaching is based on the inventors' contribution that providing the specific trans-acting apomixis effector of the present invention, in particular in an expression-increased manner, in a transformed plant cell allows the induction of an apomictic phenotype in a plant cell. Thus, in one embodiment of the present invention it is postulated to transform said plant cell with at least one plant vector comprising a nucleotide sequence coding for a transacting apomixis effector, which is preferably under control of regulatory sequences, in particular a strong constitutive or an inducible promoter, in particular allowing an increased expression in comparison to the wild type expression. Thus, such a transacting apomixis effector coding nucleotide sequence will, once transformed, integrated and expressed in a plant cell, preferably allow for the enhanced or modified expression and production of a transacting apomixis effector so as to allow, in a preferred embodiment together with a cis-acting regulatory element operably linked to a nucleotide sequence coding for a protein with the activity of a DEDDh exonuclease, the production of the desired apomictic phenotype. The cis-acting regulatory element, preferably operably linked to a nucleotide sequence coding for a protein with the activity of a DEDDh exonuclease, may be the endogenously present nucleotide sequence or may be an exogenous transgenic nucleotide sequence element itself.

In one embodiment, the introduction of the specific cis-acting regulatory elements of the present invention comprising at least one regulatory nucleotide core sequence, preferably operably linked to a DEDDh exonuclease, causes the expression of said exonuclease in the ovule of a transgenic plant obtained by the present invention and thereby provides an apomictic phenotype to the plants of the present invention. The present invention thus teaches the specific interaction of specific transacting apomixis effectors with specific cis-acting regulatory elements, in particular regulatory nucleotide core sequences of the present invention.

The present invention is essentially based on nucleic acid molecules which represent the so-called apollo gene, which means “Apomixis linked locus”, or essential and specific parts thereof. Said gene, in particular its coding sequence, codes for the apollo protein which upon expression in the plant ovule leads to the production of apomictic seed.

The present invention advantageously uses polynucleotides, in particular polynucleotides coding for a protein capable of inducing apomixis in a plant, namely the apollo protein, and polynucleotides capable of functioning as regulatory elements for said coding sequence, in isolated and purified form. Furthermore, the present invention is based on the teaching that plants, in particular their genome, comprise endogenously nucleotide sequences, hereinafter also called “polynucleotide” or “polynucleotide sequence”, coding said apollo protein capable of inducing apomixis and its regulatory elements, hereinafter also called “endogenously present polynucleotide coding a protein capable of inducing apomixis in a plant”. Thus, both the coding and the regulatory sequences as specified for instance in SEQ ID No. 37, 40, 43, 46, 49 or 52 are usually endogenously present in various allelic states in their natural and original genome environment in a plant, particularly in Brassicaceae, preferably Boechera, and are responsible for the development of a sexual or apomictic phenotype in the plant. In the naturally occurring sexually propagating plant, said nucleotide sequences in their sexual allelic state (hereinafter also termed “sex alleles”), such as in SEQ ID No. 46, 49 or 52, however, are in the ovule of said plant repressed, suppressed or not, activated or inactivated, that means not expressed, thereby preventing apomixis. In contrast, said polynucleotide in its apomictic allelic state (hereinafter also termed “apo alleles”), such as in SEQ ID No. 37, 40 or 43 is induced or derepressed, or not inactivated, that means is expressed in the ovule of a plant propagating asexually, that means an apomictic plant.

In particular, the invention is based on the teaching that in a plant ovule of a sexually propagating plant the endogenously present gene coding for the apollo protein with an apomixis-inducing capacity is suppressed, repressed, not activated or inactivated in said tissue and therefore needs to be activated in order to produce an apomictic plant. Both in sexually and apomictic plants the coding regions of the apollo gene in its apomictic and sexual allelic form, are functionally equivalent. Differences in their expression are due to their different regulatory elements, preferably as specified in SEQ ID No. 55 to 62 and 65 to 119. In particular, apomictic regulatory elements, preferably the promoter sequence given in SEQ ID No. 55, 57, 58, 59 and 107 to 119, are in particular characterised by the presence of specific promoter insertions, most preferably a regulatory nucleotide core sequence being any one of SEQ ID No. 66 to 79 which leads to an expression in the ovule of a coding element linked to said regulatory element.

The sexual regulatory element used in the present invention is in particular characterised by the absence of such a promoter insert, in particular the absence of the regulatory nucleotide core sequences specified above, e. g. of SEQ ID No. 65, in particular by the absence of the nucleotide core sequence being any one of SEQ ID No. 66 to 79. Preferably, the sexual regulatory element, preferably the promoter, is represented in particular by the presence of a regulatory element, i. e. the regulatory nucleotide target sequence having a nucleotide sequence as given in SEQ ID No. 80 to 85 and as contained in the promoter sequences given in SEQ ID No. 56, 60, 61, 62 and 86 to 106 and provides a somatic gene expression, but not an expression in the ovule, possibly due to being suppressed in said tissue.

In particular, the invention therefore provides the teaching to modify, in particular activate or induce, that means to get nucleotide sequences coding the apollo protein in an ovule expressed in order to achieve a plant of a desired phenotype, in particular an apomictic phenotype. This can preferably be achieved by transforming a plant with regulatory nucleotide core sequences of the present invention inducing the expression of a transgenic or an endogenously present polynucleotide coding for the present protein capable of inducing apomixis, that means the apollo protein in said plant.

The present invention in one preferred embodiment relates to a method according to the present invention, wherein the regulatory nucleotide core sequence contained in the cis-acting regulatory element is a transcription binding site (in the following also termed “TBS” or transcription factor binding site) for ATHB-5, LIM-1, SORLIP1AT, SORLIP2AT or POLASIG1.

The present invention in one preferred embodiment relates to a method according to the present invention, wherein the cis-acting regulatory element is a transgenic cis-acting regulatory element.

The present invention in one preferred embodiment relates to a method according to the present invention, wherein the plant cell provided in step a) is transformed in step b) with a plant vector containing an exogenous nucleotide sequence element, in particular a nucleotide sequence, comprising the cis-acting regulatory element.

The present invention in one preferred embodiment relates to a method according to the present invention, wherein the exogenous nucleotide sequence element comprising the cis-acting regulatory element additionally comprises a nucleotide sequence coding for a protein with the activity of a DEDDh exonuclease.

The present invention in one embodiment also relates to a method for the production of a transgenic apomictic plant, in particular according to the above, comprising the following steps:
m) providing a plant cell of a sexually propagating plant, which comprises a nucleotide sequence coding for a protein with the activity of a DEDDh exonuclease under control of a cis-acting regulatory element,
n) modifying the cis-acting regulatory element controlling the nucleotide sequence coding for a protein with the activity of a DEDDh exonuclease by creating at least one regulatory nucleotide core sequence to be contained in said cis-acting regulatory element and being selected from the group consisting of the ATHB-5 binding site of any one of SEQ ID No. 66 or 67, the LIM-1 binding site of any one of SEQ ID No. 68 to 73, the SORLIP1AT binding site of any one of SEQ ID No. 74 or 75, the SORLIP2AT binding site of any one of SEQ ID No. 76 or 77 and the POLASIG1 binding site of any one of SEQ ID No. 78 or 79, and
o) regenerating the plant cell obtained in step n), which contains the newly created at least one regulatory nucleotide core sequence into a transgenic plant exhibiting apomixis.

In a preferred embodiment, the cis-acting regulatory element controlling the nucleotide sequence coding for a protein with the activity of a DEDDh exonuclease contained in a plant cell of a sexually propagating plant is the wild type cis-acting regulatory element of said DEDDh exonuclease in said sexually propagating plant, in particular the cis-regulatory element of the sex apollo gene. Thus, said cis-acting regulatory elements most preferably contain at least one regulatory nucleotide target sequence, but not a regulatory nucleotide core sequence.

In the context of the present invention the term “creating at least one regulatory nucleotide core sequence” refers to the insertion or deletion of at least one nucleotide in the cis-acting regulatory element or an inversion of at least two nucleotides in said cis-acting regulatory element so as to produce, that means create, at least one regulatory nucleotide core sequence.

According to the above teaching, the cis-acting regulatory element is modified, in particular mutated, so as to contain at least one regulatory nucleotide core sequence of the present invention which in the plant cell provided in step m) was either not naturally present at said place or not present at all. Said mutation may be an insertion of one or more additional nucleotide sequences, a deletion of at least one nucleotide or an inversion of existing nucleotide sequences so as to provide in the cis-acting regulatory element at least regulatory nucleotide core sequence as identified above.

In one embodiment said modification in step n), in particular mutation, is caused by or is associated with an induced mutation, for instance the recombination, duplication, deletion, excision, insertion or inversion of all or part of a cis-regulatory element endogenously being present in a sex allele of the apollo gene and being operably linked to the coding sequence of the polypeptide capable of inducing apomixis in a plant ovule which modification allows the expression of said polypeptide consequently leading to apomixis in the plant.

The present invention in one embodiment also relates to a method for the production of a transgenic apomictic plant, in particular according to the above, comprising the following steps:
x) providing a plant cell of a sexually propagating plant, which comprises a nucleotide sequence coding for a protein with the activity of a DEDDh exonuclease under control of a cis-acting regulatory element,
y) modifying the cis-acting regulatory element controlling the nucleotide sequence coding for a protein with the activity of a DEDDh exonuclease by mutating, for instance deleting, at least one regulatory nucleotide target sequence contained in said cis-acting regulatory element and being selected from the group consisting of any one of SEQ ID No. 80 to 85 and
z) regenerating the plant cell obtained in step y), which contains the deletion of said at least one regulatory nucleotide target sequence, into a transgenic plant exhibiting apomixis.

In a preferred embodiment, the cis-acting regulatory element controlling the nucleotide sequence coding for a protein with the activity of a DEDDh exonuclease contained in a plant cell of a sexually propagating plant is the wild type cis-acting regulatory element of said DEDDh exonuclease in said sexually propagating plant, in particular the cis-regulatory element of the sex apollo gene. Thus, said cis-acting regulatory elements most preferably contain at least one regulatory nucleotide target sequence, but not a regulatory nucleotide core sequence.

In the context of the present invention the term “mutating at least one regulatory nucleotide target sequence” refers to the insertion or deletion of at least one nucleotide in the regulatory nucleotide target sequence or an inversion of at least two nucleotides in said regulatory nucleotide target sequence. Mutating the at least one regulatory nucleotide target sequence therefore has the effect that the nucleotide sequence as a result of said mutation is different in at least one nucleotide with regard to the original at least one regulatory nucleotide target sequence present in the plant cell provided in step x).

In one embodiment said modification performed in step y), in particular mutation, is caused by or associated with an induced mutation, for instance a recombination, duplication, deletion, excision, insertion or inversion, of all or part of a regulatory nucleotide target sequence endogenously being present in a sex allele of the apollo gene and being operably linked to the coding sequence of the polypeptide capable of inducing apomixis in a plant ovule which modification allows the expression of said polypeptide consequently leading to apomixis in the plant.

The present invention thus allows and enables the induction of apomixis in a plant by modifying, in particular inducing, hereinafter also called activating, the expression of the endogenously present regulatory elements of the endogenously present nucleotide sequence encoding a protein capable of inducing apomixis in a sexual plant by structurally modifying said endogenously present regulatory nucleotide target sequence for instance by mutating, in particular by excision, insertion, duplication or inversion of said regulatory nucleotide target sequence so as to completely delete it or remove it to another genomic location. Said structural modification may preferably be achieved by any means for mutation, for instance radiation, use of chemical agents or of nucleotide sequences, in particular a DNA molecule, introduced into a plant cell, which means, in particular sequence, is capable of structurally interfering with said regulatory nucleotide target sequence and which sequence may be a transposon or any other sequence being able to interfere, for instance recombine or insert into said regulatory nucleotide target sequence in the ovule of a sexually propagating plant.

In one further embodiment of the present invention a method is provided for the production of a transgenic apomictic plant, in particular according to the above, comprising above-identified process steps x), y), n) and z). Thus, in this embodiment the cis-acting regulatory element controlling the nucleotide sequence coding for a protein with the activity of a DEDDh exonuclease is mutated by creating at least one regulatory nucleotide core sequence and by deleting at least one regulatory nucleotide target sequence such as identified in the present invention.

Thus, the present invention postulates in one embodiment the interruption, that means in particular deletion, of a binding site for a suppressor in sexual ovules by mutational processes so as to cause said site not any more recognised by said suppressor, but in a preferred embodiment by an activator in ovules of plants, thus becoming apomictic of the reproductive process. In one embodiment the interruption of a suppressor binding site, preferably a regulatory nucleotide target sequence, is sufficient to produce from a sexual apollo allele an apomictic apollo allele. In another embodiment, the creation of an activator binding site, preferably a regulatory nucleotide core sequence, in a sexual apollo allele so as to create an apomictic apollo allele is sufficient to obtain an apomictic phenotype. In another embodiment, both interruption of a suppressor binding site and creation of an activator binding site, optionally both of said sites being at the same location, is sufficient to produce an apomictic phenotype. In another embodiment, the interruption of the suppressor binding site and the creation of the activator binding site occurs at different sites of the cis-regulatory element of the present invention.

The present invention in one preferred embodiment relates to a method according to the present invention, wherein the nucleotide target sequence contained in the cis-acting regulatory element of a sex apollo allele is a transcription binding site for Dof2, Dof3 or PBF.

The present invention in one preferred embodiment relates to a method according to the present invention, wherein the plant cell provided in step a), x) or m) is transformed with a plant vector containing an exogenous nucleotide sequence element comprising a nucleotide sequence encoding a trans-acting apomixis effector.

In a particularly preferred embodiment, the exogenous nucleotide sequence encoding the transacting apomixis effector comprises a regulatory element, in particular a promoter controlling the expression of said transacting apomixis effector, in particular comprises a promoter providing for a high efficiency, constitutive or inducible expression of said effector.

The present invention in one preferred embodiment relates to a method according to the present invention, wherein the trans-acting apomixis effector is an over-expressed trans-acting apomixis effector.

The present invention in one preferred embodiment relates to a method according to the present invention, wherein the trans-acting apomixis effector is a transcription factor, in particular ATHB-5, LIM-1, SORLIP1AT, SORLIP2AT or POLASIG1. In a furthermore preferred embodiment of the present invention the transcription factor is a genetically modified transcription factor for instance providing an enhanced transcription efficiency.

The present invention in one preferred embodiment relates to a method according to the present invention, wherein the nucleotide sequence coding for a protein with the activity of a DEDDh exonuclease comprises a nucleotide sequence selected from the group consisting of al) the polynucleotide defined in any one of SEQ ID No. 22 to 54, or a fully complementary strand thereof, in particular of any one of SEQ ID No. 23, 25, 27, 28, 29, 30, 33, 35, 37, 38, 40, 41, 43, 44, 47, 50 or 53, or a fully complementary strand thereof, b1) a polynucleotide encoding a polypeptide with the amino acid sequence defined in any one of SEQ ID No. 1 to 21 or a fully complementary strand thereof, preferably of any one of SEQ ID No. 4 to 9, SEQ ID No. 13 to 15 or SEQ ID No. 19 to 21, or a fully complementary strand thereof, and c1) a polynucleotide variant having a degree of sequence identity of more than 70% to the nucleic acid sequence defined in a1) or b1) of a fully complementary strand thereof, preferably wherein the sequence identity is based on the entire sequence and is determined by BLAST analysis, preferably in the NCBI database, in particular by GAP analysis using Gap Weight of 50 and Length Weight of 3.

The present invention in one preferred embodiment relates to a method according to the present invention, wherein the nucleotide sequence coding for a protein with the activity of a DEDDh exonuclease comprises a nucleotide sequence selected from the group consisting of a2) the polynucleotide defined in any one of SEQ ID No. 22, 23, 27, 28, 32, 33 or a fully complementary strand thereof, preferably any one of SEQ ID No. 23, 28 or 33, or a fully complementary strand thereof, b2) a polynucleotide encoding a polypeptide with the amino acid sequence defined in any one of SEQ ID No. 4, 5, 6 or a fully complementary strand thereof, and c2) a polynucleotide variant having a degree of sequence identity of more than 70% to the nucleic acid sequence defined in a2) or b2) or a fully complementary strand thereof, preferably wherein the sequence identity is based on the entire sequence and is determined by BLAST analysis, preferably in the NCBI database, in particular by GAP analysis using Gap Weight of 50 and Length Weight of 3.

The present invention also uses in a preferred embodiment the above-identified polynucleotide coding for a protein with the activity of a DEDDh exonuclease which is in particular characterised by the presence of at least one specific duplicated marker sequence in an exon, namely the fifth exon, of said sequence and which represents a nucleotide stretch duplication. Preferably, said duplicated marker nucleotide sequence is given in SEQ ID No. 64 and its corresponding amino acid sequence in SEQ ID No. 63.

The present invention in an embodiment also relates to a method for identifying an apomixis effector in a plant, wherein a nucleotide sequence selected from the group consisting of the ATHB-5 binding site of any one of SEQ ID No. 66 or 67, the LIM-1 binding site of any one of SEQ ID No. 68 to 73, the SORLIP1AT binding site of any one of SEQ ID No. 74 or 75, the SORLIP2AT binding site of any one of SEQ ID No. 76 or 77 and the POLASIG1 binding site of any one of SEQ ID No. 78 or 79 is used in a DNA-protein-binding assay so as to identify proteins binding to said nucleotide sequences.

The present invention in an embodiment also relates to a transgenic apomictic plant produced according to any one of the present methods.

The present invention in an embodiment also relates to a transgenic plant material from a plant according to the above.

The “regulatory nucleotide core sequence” of the present invention which presence is useful for the generation of the desired apomictic phenotype is in a preferred embodiment a transcription factor binding site, in particular a transcription binding site, and is particularly preferred selected from the group consisting of binding sites for ATHB-5, LIM-1, SORLIP1AT, SORLIP2AT and POLASIG1. Thus, said regulatory nucleotide core sequences are located in the cis-acting regulatory element of a nucleotide sequence coding for a protein with the activity of a DEDDh exonuclease and are in even more preferred embodiments located in the following specifically identified positions. These positions are given herein with regard to SEQ ID No. 27.

In a preferred embodiment, the ATHB-5 transcription binding site (SEQ ID No. 66 and 67) is located within the cis-regulatory sequence and with reference to SEQ ID No. 27 at position 62 to 70 in the (+) (in the following also termed “sense” or “positive” strand) strand.

In a particularly preferred embodiment, the LIM-1 transcription binding site (SEQ ID No. 68, 69 and 70) is located in the cis-regulatory sequence and with reference to SEQ ID No. 27 in the (+) strand at position 43 to 54. Most preferably, the LIM-1 transcription binding site is located in the (−) strand (in the following also termed “anti-sense” or “negative” strand) and is represented by SEQ ID No. 71, 72 or 73.

In a furthermore preferred embodiment, the SORLIP1AT transcription binding site (SEQ ID No. 74) is located within the cis-regulatory sequence and with reference to SEQ ID No. 27 at position 51 to 55 in the (+) strand. Most preferably, the SORLIP1AT transcription binding site is present in the (−) strand and is presented by SEQ ID No. 75.

In a furthermore preferred embodiment, the SORLIP2AT transcription binding site (SEQ ID No. 76) is located within the cis-regulatory sequence and with regard to SEQ ID No. 27 at position 53 to 57 in the (+) strand. Most preferably the SORLIP2AT transcription binding site is present in the (−) strand and is represented by SEQ ID No. 77.

In a furthermore preferred embodiment, the POLASIG1 transcription binding site (SEQ ID No. 78) is located within the cis-regulatory sequence and with regard to SEQ ID No. 27 at position 64 to 69 in the (+) strand. Most preferably, the POLASIG1 transcription binding site is present in the (−) strand and is represented by SEQ ID No. 79.

In another embodiment of the present invention it is postulated to modify a sexually propagating plant, in particular a sex allele of the apollo gene, so as to mutate, in particular interrupt, delete or functionally inactivate a transcription factor binding site, in particular a transcription binding site, present in a cis-acting regulatory element of a nucleotide sequence coding for a protein with activity of a DEDDh exonuclease and wherein said binding site is hereinafter also termed a “regulatory nucleotide target sequence” which is preferably selected from the group consisting of transcription factor binding sites for the transcription factors Dof2, Dof3 and PBF. Preferably, said regulatory nucleotide target sequence is mutated, preferably interrupted, preferably deleted, in a sex allele so as to produce an apo allele. Most preferably, the position of said deletion with regard to SEQ ID No. 32 is given in the following.

The nucleotide target sequence contained in the sex alleles and to be interrupted to obtain apo alleles are present, given in relation to SEQ ID No. 32, in case of Dof2 (SEQ ID No. 80) at position 59 to 69, preferably on the (−) strand (SEQ ID No. 81), in case of Dof3 (SEQ ID No. 82) at position 60 to 65, preferably on the (−) strand (SEQ ID No. 83), and in case of PBF (SEQ ID No. 84) at position 61 to 65, preferably on the (−) strand (SEQ ID No. 85).

The present inventors identified said cis-regulatory elements and revealed that the promoter of the apollo gene containing an apomixis-specific polymorphism (TGGCCCGTGAAGTTTATTCC) (SEQ ID No. 65) is characterized on the (+) strand by a transcription binding site (agtTTATTc) (SEQ ID No. 67) for the ATHB-5 transcription factor which is absent in all sex alleles. The same polymorphism generates in the (−) strand TBSs for Lim1 (aagaggaGGTGG) (SEQ ID No. 70), SORLIP1AT (GTGGC) (SEQ ID No. 74), SORLIP2AT (GGCCC) (SEQ ID No. 76) and POLASIG1 (TTTATT) (SEQ ID No. 78). Sex alleles of the present invention contain in that region on the (−) strand TBSs for Dof2/Dof3 (ttGCTTTaaaa (SEQ ID No. 80) and TGCTTT (SEQ ID No. 82)) and PBF (GCTTT) (SEQ ID No. 84). The upper case letters in the above represent invariable nucleotides, while the lower case letters represent variable nucleotides.

Without being bound by theory, it appears that in sexual Boechera genotypes the apollo gene is actively expressed or derepressed in any allelic form in leaves but it is specifically repressed or not activated in ovules entering meiosis; and in apomictic Boechera, the apollo gene is as well actively expressed or derepressed in any allelic form in leaves but it is not repressed or inactivated in ovules entering apomeiosis due to the presence of a polymorphism in the 5′ UTR. Sequence analysis for transcription factor binding sites on the 5′ UTR region revealed that the polymorphism contains, fully or partially, specific TBSs for the ATHB-5, LIM1, SORLIP1AT, SORLIP2AT and POLASIG1 transcription factors in apo alleles. Instead, in sex alleles the region occupied by the apospecific polymorphism, contain specific TBSs for Dof2, Dof3 and PBF transcription factors.

ATHB-5 is a class I HDZip (homeodomain-leucine zipper) protein that is a positive regulator of ABA-responsiveness mediating the inhibitory effect of ABA on growth during seedling establishment. ATHB-5 has been also found to be maternally expressed when analyzing cDNA-AFLP on A. thaliana siliques.

LIM1 is a widespread transcription factor being already detected in many model plants like Glycine max, Lotus japonicus, Nicotiana tabacum and A. thaliana which function is still not well known.

SORLIP1AT and SORLIP2AT are sequences over-represented in light-induced promoters in arabidopsis. SORLIP1 is the most over-represented and seems to be strand-independent.

POLASIG1 sequence is a canonical nucleotide sequence (AAUAA) highly conserved across the majority of pre-mRNA. This is a signal for the cleavage and polyadenylation specificity factor (CPSF) which is involved in the cleavage of the 3′ signaling region from a pre-mRNA. This target is senseful for an ORF lying on the negative strand.

Dof (DNA-binding one finger) is a family of plant proteins that share a highly conserved and unique DNA binding domain with one Cys2/Cys2 zinc finger motif. Many gene promoters have been already associated with Dof proteins but their regulation mechanisms and physiological functions remain elusive. In maize, Dof2 is mainly expressed in leaves, stems and roots, and it has been shown to act as a transcriptional repressor. In rice, OsDof3 is specifically expressed in the scutellum and the endosperm in response to gibberellic acid (GA) during germination. In Arabidopsis, the maternally expressed AtDof3.7 is involved in the control of seed germination.

PBF (Prolamin box binding factor) binding activity has been detected in maize endosperm nuclei, and in combination with the leucine zipper (bZIP) transcription factor Opaque 2 (O2), it is important in the regulation of 22-kDa zein gene expression (which mRNA and protein expression is limited to the endosperm).

Thus, the present invention provides advantageous means and methods to induce apomixis in a plant. The polynucleotides used in the present invention, in particular those which code for a protein capable of inducing apomixis, can be used to be transformed in a plant cell so as to produce a plant which comprises said exogenously introduced polynucleotide, expresses said polynucleotide in a plant ovule and thereby produces an apomictic phenotype and apomictic plant. This can in a particularly preferred embodiment be achieved by using the polynucleotides, preferably defined in any one of SEQ ID No. 22 to 54, preferably 23, 25, 27, 28, 29, 30, 33, 35, 37, 38, 40, 41, 43, 44, 47, 50 or 53, in particular 23, 25, 28, 30, 33, 35, 38, 41, 44, 47, 50 or 53, coding for a protein capable of inducing apomixis in a plant ovule, preferably defined in any one of SEQ ID No. 4 to 21, preferably SEQ ID No. 4 to 9, SEQ ID No. 13 to 15 or SEQ ID No. 19 to 21, under control of a promoter providing an expression in the ovule due to the presence of a cis-regulatory element comprising at least one regulatory core sequence of the present invention.

Thus, in one preferred aspect of the present invention the isolated nucleic acid molecules comprise polynucleotides, in particular polynucleotides as specifically disclosed herein or polynucleotide variants, for use in inducing apomixis, which code for a protein capable of inducing apomixis in a plant, in particular in a plant ovule, in particular code for a protein with a specific exonuclease activity capable of inducing apomixis, in particular apomeiosis, in a plant ovule, and wherein said specific polynucleotides variants thereof can advantageously be used to be transferred into a plant, in particular plant cell, be stably integrated in its genome and can preferably be expressed in the ovule of the obtained transformed plant in order to produce a transgenic apomictic transgenic plant, which produces apomictic seed. In a preferred embodiment of the present invention it is postulated to transfer a polynucleotide encoding a protein capable of inducing apomixis in a plant and being specified in any one of the consensus SEQ ID No. 1 to 9, preferably SEQ ID No. 4 to 9, most preferably SEQ ID No. 4 or 7, most preferably SEQ ID No. 5 or 8, most preferably SEQ ID No. 6 or 9 and in particular as specified in any one of the specific SEQ ID No. 10 to 21, preferably SEQ ID No. 13 to 15 or 19 to 21, into a plant so as to allow expression of said polynucleotide under control of an promoter allowing expression in the ovule, which comprises a cis-regulatory element comprising at least one regulatory core sequence of the present invention, thereby producing the desired apollo protein in the ovule.

The present invention also provides polynucleotides which are capable of functioning as a regulatory element, preferably a cis-regulatory element, and which can be used to transform plant cells and whereby said polynucleotides capable of functioning as regulatory elements structurally modify the regulatory elements of the endogenously present genes which code for proteins capable of inducing apomixis so as to derepress, that means activate, the endogenously present regulatory elements of said genes thereby allowing the expression of the protein capable of inducing apomixis and producing plants with an apomictic phenotype. This particular approach is based on the findings of the present invention that the gene coding for the protein capable of inducing apomixis is present also in wild type plants, but is, however, not activated, that means is not induced and therefore is not expressed in the ovule of a sexually propagating plant. Without being bound by theory, in wild type sexually propagating plants the expression of the endogenously present gene coding for a protein capable of inducing apomixis is suppressed or inactivated, most likely due to suppressed regulatory elements of the protein-coding regions. Thus, the present invention teaches in one embodiment the introduction of regulatory elements, in particular nucleotide sequences, preferably DNA molecules comprising, preferably consisting of, the present regulatory nucleotide core sequences of any one of SEQ ID No. 66 to 79, which structurally interfere with the endogenously present and suppressed regulatory elements of a nucleotide sequence region coding for a protein capable of inducing apomixis in a plant ovule allows the reversion of the suppression of the regulatory elements and induces the expression of the coding sequence.

Accordingly, the present invention uses isolated nucleic acid molecules, which comprise polynucleotides, that means the polynucleotides specifically disclosed herein, for use in inducing apomixis, wherein the specific polynucleotides represent or comprise or consist of regulatory elements, in particular the present regulatory nucleotide core sequences of any one of SEQ ID No. 66 to 79, and are useful for inducing apomixis in a plant in so far as they allow a regulatable expression of coding sequences operably linked thereto in the plant ovule, in particular during ovule development in a plant. Thus, these regulatory nucleotide core sequences provide a non-suppressability to a coding sequence in the plant ovule and provide the advantage of being capable to direct expression of coding sequences in the ovule of plants.

In a further embodiment, the present invention uses these specific polynucleotides which are capable of acting as regulatory nucleotide core sequences, in particular in case of being part of a promoter, such as identified in any one of SEQ ID No. 55, 57, 58, 59 or 107 to 119, which very specifically act in a regulatory manner in the ovule. In one preferred embodiment of such a promoter, hereinafter also called apo-promoter, of the present invention, said regulatory nucleotide core sequence causes the promoter to be expressed in the ovule of said plant.

Thus, the present invention very advantageously allows the vegetative production of seed identical to the parent. In particular and preferably, the present nucleotide acid molecules can be transformed into a desired plant, for instance high yielding hybrids, in order to change their reproductive mode into apomictic seed production. Thus, high yielding hybrids can according to the present invention be used in seed production to multiply identical copies of said high yielding hybrid seed which would greatly reduce the cost for the seed production and in turn increases the number of genotypes which could commercially be offered. Further on, genes can be evaluated directly in commercial hybrids, since the progeny would not segregate saving the cumbersome backcrossing procedures. Apomixis can be used to stabilise desirable phenotypes even with complex traits such as hybrid vigor. Such traits can be maintained very easily and be multiplied via apomixis indefinitive. Further, the present invention provides the possibility to combine it with male sterility, advantageously preventing genetically engineered stabilised traits from being hybridised with undesired relatives.

The present invention provides a solution to the above-identified technical problem by providing specific isolated nucleic acid molecules which can be used for inducing apomixis in a plant, in particular in a plant ovule, preferably for inducing apomeiosis and/or parthenogenesis in a plant, preferably in a plant ovule.

The nucleic acid molecules for use in the present invention comprise in one preferred embodiment specific polynucleotides characterised by their ability to induce apomixis in a plant and by the presence of specific consensus nucleotide sequence patterns according to any one of SEQ ID No. 27, 28, 29, 30 or 31, in particular 27, 28, 29, 30, preferably 27 or 29, which represent nucleotide patterns present in all specifically disclosed apomixis-inducing alleles of the present invention.

In a further preferred embodiment the specific polynucleotides are the various apomixis-inducing alleles, which are specifically used according to the present invention and are characterised in any one of SEQ ID No. 37 to 45.

The present invention is preferably characterised by using polynucleotides and polypeptides in specific and in consensus forms. The consensus forms are generalised sequence motifs, that means patterns, being in one embodiment found in all of the polymorphic apollo genes identified and isolated according to the present invention, in particular are common to the coding sequence of all the different polymorphic forms including the apomictic and sexual forms. The consensus sequences are also given as generalised sequence motifs solely found in the apomictic polymorphic alleles or, in another embodiment, are solely found in the sexual polymorphic allelic forms isolated. The apomictic and sexual alleles can be classified by different consensus sequences for their regulatory elements and share the same, similar or equivalent consensus sequence for their coding regions. In the consensus sequence “Xaa” stands for any naturally occurring amino acid and “n” for any one of the nucleotides a, t, g or c.

The specific polynucleotides and polypeptides used in the present invention are specifically isolated and analysed and display the consensus sequence pattern in exemplified form.

In a particularly preferred embodiment the present invention therefore uses consensus and specific polynucleotides and polypeptides characterised in the following tables I to III.

TABLE I
Apollo-amino acid sequences (polypeptides)

| SEQ | | | | coded by |
|---|---|---|---|---|
| ID No. | type | subtype | characterisation | SEQ ID No. |
| 1 | consensus | Global | Exonuclease domain | 26 |
| 2 | consensus | Apo | Exonuclease domain | 31 |
| 3 | consensus | Sex | Exonuclease domain | 36 |
| 4 | consensus | Global | protein with duplication | 22, 23 |
| 5 | consensus | Apo | protein with duplication | 27, 28 |
| 6 | consensus | Sex | protein with duplication | 32, 33 |
| 7 | consensus | Global | protein without duplication | 24, 25 |
| 8 | consensus | Apo | protein without duplication | 29, 30 |
| 9 | consensus | Sex | protein without duplication | 34, 35 |
| 10 | specific | Apo | A011a Exonuclease | 39 |
| | | | domain | |
| 11 | specific | Apo | A043a Exonuclease | 42 |
| | | | domain | |
| 12 | specific | Apo | A081a Exonuclease | 45 |
| | | | domain | |
| 13 | specific | Apo | A011a Protein | 37, 38 |
| 14 | specific | Apo | A043a Protein | 40, 41 |
| 15 | specific | Apo | A081a Protein | 43, 44 |
| 16 | specific | Sex | S011a Exonuclease | 48 |
| | | | domain | |
| 17 | specific | Sex | S355a Exonuclease | 51 |
| | | | domain | |
| 18 | specific | Sex | S390a Exonuclease | 54 |
| | | | domain | |
| 19 | specific | Sex | S011a Protein | 46, 47 |
| 20 | specific | Sex | S355a Protein | 49, 50 |
| 21 | specific | Sex | S390a Protein | 52, 53 |

legend: A011a, A043a, A081a: apomictic Boechera holboellii alleles; S011a, S355a, S390a: sexual Boechera holboellii alleles
“consensus” means consensus sequence, that means a general sequence motif present in more than one specific allele of the apollo gene with specifically identified positions for observed sequence deviations, namely nucleotide/amino acid polymorphisms. In amino acid sequences “Xaa” can be any naturally occurring amino acid. In nucleotide sequences “n” can be any of a, g, t or c, in introns “n” can additionally designate a missing nucleotide.
“specific” means a specifically isolated polymorphic allele with sequenced or deduced nucleotide and amino acid sequence.
“Global” means a consensus sequence both for apomictic and sexual apollo gene or protein.
“Apo” means apomictic apollo gene or protein.
“Sex” means sexual apollo gene or protein.
“protein” means apollo protein.
“Exonuclease domain” means the fragment of the apollo protein in which the specific biologically active DEDDh 3′-5′ exonuclease activity is located.
“duplication” means a duplicated marker sequence optionally present in the coding region of the apomictic and sexual allele of the apollo gene and specified in SEQ ID No. 63 (amino acid) and 64 (nucleotide).

TABLE II
Apollo-protein coding polynucleotides

| SEQ | | | |
|---|---|---|---|
| ID No. | type | subtype | characterisation |
| 22 | consensus | Global | genomic with duplication |
| 23 | consensus | Global | coding with duplication |
| 24 | consensus | Global | genomic without duplication |
| 25 | consensus | Global | coding without duplication |
| 26 | consensus | Global | Exonuclease domain |
| 27 | consensus | Apo | genomic with duplication |
| 28 | consensus | Apo | coding with duplication |
| 29 | consensus | Apo | genomic without duplication |
| 30 | consensus | Apo | coding without duplication |
| 31 | consensus | Apo | Exonuclease domain |
| 32 | consensus | Sex | genomic with duplication |
| 33 | consensus | Sex | coding with duplication |
| 34 | consensus | Sex | genomic without duplication |
| 35 | consensus | Sex | coding without duplication |
| 36 | consensus | Sex | Exonuclease domain |
| 37 | specific | Apo | A011a genomic |
| 38 | specific | Apo | A011a coding |
| 39 | specific | Apo | A011a Exonuclease domain |
| 40 | specific | Apo | A043a genomic |
| 41 | specific | Apo | A043a coding |
| 42 | specific | Apo | A043a Exonuclease domain |
| 43 | specific | Apo | A081a genomic |
| 44 | specific | Apo | A081a coding |
| 45 | specific | Apo | A081a Exonuclease domain |
| 46 | specific | Sex | S011a genomic |
| 47 | specific | Sex | S011a coding |
| 48 | specific | Sex | S011a Exonuclease domain |
| 49 | specific | Sex | S355a genomic |
| 50 | specific | Sex | S355a coding |
| 51 | specific | Sex | S355a Exonuclease domain |
| 52 | specific | Sex | S390a genomic |
| 53 | specific | Sex | S390a coding |
| 54 | specific | Sex | S390a Exonuclease domain |

legend: see table I; “genomic” means genomic DNA sequence, preferably including regulatory elements, exons and introns.
“coding” means solely the coding DNA sequence which codes the full length apollo protein.

TABLE III
Apollo-regulatory polynucleotides, peptides and inserts

| SEQ | | | |
|---|---|---|---|
| ID No. | type | subtype | characterisation |
| 55 | consensus | Apo | promoter |
| 56 | consensus | Sex | promoter |
| 57 | specific | Apo | A011a promoter |
| 58 | specific | Apo | A043a promoter |
| 59 | specific | Apo | A081a promoter |
| 60 | specific | Sex | S011a promoter |
| 61 | specific | Sex | S355a promoter |
| 62 | specific | Sex | S390a promoter |
| 63 | specific | Apo/Sex | duplication, amino acids |
| 64 | specific | Apo/Sex | duplication, DNA |
| 65 | specific | Apo | promoter insert |
| 66 | specific | Apo | ATHB-5 binding (+) |
| 67 | more specific | Apo | ATHB-5 binding (+) |
| 68 | specific | Apo | LIM-1 binding (+) |
| 69 | more specific | Apo | LIM-1 binding (+) |
| 70 | most specific | Apo | LIM-1 binding (+) |
| 71 | specific | Apo | LIM-1 binding (−) |
| 72 | more specific | Apo | LIM-1 binding (−) |
| 73 | most specific | Apo | LIM-1 binding (−) |
| 74 | specific | Apo | SORLIP1AT binding (+) |
| 75 | specific | Apo | SORLIP1AT binding (−) |
| 76 | specific | Apo | SORLIP2AT binding (+) |
| 77 | specific | Apo | SORLIP2AT binding (−) |
| 78 | specific | Apo | POLASIG1 binding (+) |
| 79 | specific | Apo | POLASIG1 binding (−) |
| 80 | specific | Sex | Dof2 binding (+) |
| 81 | specific | Sex | Dof2 binding (−) |
| 82 | specific | Sex | Dof3 binding (+) |
| 83 | specific | Sex | Dof3 binding (−) |
| 84 | specific | Sex | PBF binding (+) |
| 85 | specific | Sex | PBF binding (−) |
| 86 | specific | Sex | 329S2_S1 promoter |
| 87 | specific | Sex | 33A2_S6 promoter |
| 88 | specific | Sex | 385S2_S3 promoter |
| 89 | specific | Sex | 385S2_S11 promoter |
| 90 | specific | Sex | 390S2_S16 promoter |
| 91 | specific | Sex | 390S2_S1 promoter |
| 92 | specific | Sex | 1A2_S6 promoter |
| 93 | specific | Sex | 344S7_S2 promoter |
| 94 | specific | Sex | 111A2_S13 promoter |
| 95 | specific | Sex | 43A3_S4 promoter |
| 96 | specific | Sex | 215A3_S13 promoter |
| 97 | specific | Sex | 104A3_S7 promoter |
| 98 | specific | Sex | 355S2_S3 promoter |
| 99 | specific | Sex | 376S2_S5 promoter |
| 100 | specific | Sex | 369S2_S3 promoter |
| 101 | specific | Sex | 66A3_S8 promoter |
| 102 | specific | Sex | 168A2_S4 promoter |
| 103 | specific | Sex | 380S2_S13 promoter |
| 104 | specific | Sex | 215A3_S5 promoter |
| 105 | specific | Sex | 11A2_S8 promoter |
| 106 | specific | Sex | 1A2_S7 promoter |
| 107 | specific | Apo | 33A2_A5 promoter |
| 108 | specific | Apo | 168A2_A6 promoter |
| 109 | specific | Apo | 1A2_A3 promoter |
| 110 | specific | Apo | 11A2_A5 promoter |
| 111 | specific | Apo | 111A2_A8 promoter |
| 112 | specific | Apo | 43A3_A7 promoter |
| 113 | specific | Apo | 215A3_A7 promoter |
| 114 | specific | Apo | 104A3_A4 promoter |
| 115 | specific | Apo | 43A3_A3 promoter |
| 116 | specific | Apo | 66A3_A3 promoter |
| 117 | specific | Apo | 1A2_A6 promoter |
| 118 | specific | Apo | 11A2_A3 promoter |
| 119 | specific | Apo | 11A2_A1 promoter |

legend: see table I; “promoter insert”: regulatory insertion of 20 bp found in apo-promoters; (+): positive (sense) strand; (−): negative (anti-sense) strand.

The present invention uses in one embodiment global consensus genomic sequences, in particular those of SEQ ID No. 22 and 24 which represent nucleotide sequence patterns found in the apomictic and sexual alleles in so far as the nucleotide sequences given are to be found in both types of alleles.

Thus, in a particularly preferred embodiment of the present invention polynucleotides coding for the apollo protein are used which are characterised by any one of the polynucleotide sequences given in SEQ ID No. 23, 25 to 31, 33, 35 to 45, 47, 48, 50, 51, 53 or 54 which are consensus and specific sequences found in apomictic and sexual alleles and which code for the consensus or specific apollo protein used in the present invention of any one of SEQ ID No. 1 to 21, preferably of SEQ ID No. 4 to 9, 13 to 15 or 19 to 21 or an essential part thereof, namely the exonuclease domain of SEQ ID No. 1 to 3, 10 to 12 or 16 to 18. Most preferred are polynucleotides identified in Table I coding for the consensus apollo proteins or essential parts thereof, namely any one of SEQ ID No. 1 to 21, preferably 4, 5, 6, 7, 8, 9, 13, 14, 15, 19, 20 or 21, in particular 4, 5, 6, 7, 8 or 9.

The present invention also uses functionally equivalent polynucleotides for inducing apomixis in a plant, in particular in a plant ovule, preferably for inducing apomeiosis and/or parthenogenesis in a plant, preferably in a plant ovule, which do not exactly show the specific nucleotide sequence of said specific nucleotide sequence patterns or apomixis-inducing alleles and in particular given in the sequence identity protocols given herein, but which do exhibit slight deviations therefrom and which are in the context of the present invention termed “polynucleotide variants”. Such polynucleotide variants are allelic, polymorphic, mutated, truncated or prolonged variants of the polynucleotides defined in the present sequence identity protocols and which therefore show deletions, insertions, inversions or additions of nucleotides in comparison to the polynucleotides defined in the present sequence identity protocol. Thus, polynucleotide or polypeptide variants of the present invention, hereinafter also termed “functional equivalents” of a polynucleotide or polypeptide, have a structure and a sufficient length to provide the same biological activity, that means the same capability to induce apomixis in the plant as the specifically disclosed polynucleotides or polypeptides of the present invention.

A polypeptide coded by a polynucleotide variant used in the present invention is—in case its amino acid sequence is altered in comparison to the amino acid sequence of the polypeptide coded by the polynucleotide of the present invention—termed a polypeptide variant. However, due to the degeneracy of the genetic code a polynucleotide variant not necessarily codes in any case for a polypeptide variant but may also code a polypeptide of the present invention.

The term “variant” refers to a substantially similar sequence of the specifically disclosed polynucleotides or polypeptides used in the present invention. Generally, polynucleotide variants of the invention will have at least 60%, 65%, or 70%, preferably 75%, 80% or 90%, more preferably at least 95% and most preferably at least 98% sequence identity to the present polynucleotides, in particular those representing the present apomixis-inducing alleles, in particular its coding sequence, wherein the % sequence identity is based on the entire sequence and is determined by BLAST analysis, preferably in the NCBI database, in particular by GAP analysis using Gap Weight of 50 and Length Weight of 3.

Generally, polypeptide sequence variants used in the invention will have at least about 50%, 55%, 60%, 65%, 70%, 75% or 80%, preferably at least about 85% or 90%, and more preferably at least about 95% sequence identity to the present protein capable of inducing apomixis, wherein the % sequence identity is based on the entire sequence and is determined by BLAST analysis, preferably in the NCBI database, in particular by GAP analysis using Gap Weight of 12 and Length Weight of 4.

According to the present invention a number of amino acids of the present polypeptides can be replaced, inserted or deleted without altering a protein's function. The relationship between proteins is reflected by the degree of sequence identity between aligned amino acid sequences of individual proteins or aligned component sequences thereof.

Dynamic programming algorithms yield different kinds of alignments. Algorithms as proposed by Needleman and Wunsch and by Sellers align the entire length of two sequences providing a global alignment of the sequences. The Smith-Waterman algorithm yields local alignments. A local alignment aligns the pair of regions within the sequences that are most similar given the choice of scoring matrix and gap penalties. This allows a database search to focus on the most highly conserved regions of the sequences. It also allows similar domains within sequences to be identified. To speed up alignments using the Smith-Waterman algorithm both BLAST (Basic Local Alignment Search Tool) and FASTA place additional restrictions on the alignments.

Within the context of the present invention alignments are conveniently performed using BLAST, a set of similarity search programs designed to explore all of the available sequence databases regardless of whether the query is protein or DNA. Version BLAST 2.2 (Gapped BLAST) of this search tool has been made publicly available (currently http World Wide Web internet site “ncbi.nlm.nih.gov/BLAST” or http World Wide Web internet site “blast.ncbi.nlm.nih.gov/BLAST.cgi”). It uses a heuristic algorithm which seeks local as opposed to global alignments and is therefore able to detect relationships among sequences which share only isolated regions. The scores assigned in a BLAST search have a well-defined statistical interpretation. Particularly useful within the scope of the present invention are the blastp program allowing for the introduction of gaps in the local sequence alignments and the PSI-BLAST program, both programs comparing an amino acid query sequence against a protein sequence database, as well as a blastp variant program allowing local alignment of two sequences only.

Sequence alignments using BLAST can also take into account whether the substitution of one amino acid for another is likely to conserve the physical and chemical properties necessary to maintain the structure and function of a protein or is more likely to disrupt essential structural and functional features. For example non-conservative replacements may occur at a low frequency and conservative replacements may be made between amino acids within the following groups: (i) serine and threonine; (ii) glutamic acid and aspartic acid; (iii) arginine and lysine; (iv) asparagine and glutamine; (v) isoleucine, leucine, valine and methionine; (vi) phenylalanine, tyrosine and tryptophan (vii) alanine and glycine.

Such sequence similarity is quantified in terms of percentage of positive amino acids, as compared to the percentage of identical amino acids.

The polynucleotide or polypeptide variants used in the present invention, however, are in spite of their structural deviations also capable of exhibiting the same or essentially the same biological activity as the polynucleotides or polypeptides defined in the sequence identity protocols of the present invention.

In the context of the present invention the term “biological activity” refers to the capability of the polynucleotide or polypeptide of the present invention or their variants to induce apomixis in a plant. The term “to induce apomixis in a plant” refers to the capability of a polynucleotide or polypeptide or variant thereof to induce an asexual production of viable seed in a plant, in particular in the ovule of a plant, in particular the capability to induce apomeiosis or parthenogenesis or both apomeiosis and parthenogenesis in a plant ovule, in particular by coding or exerting an exonuclease activity in the ovule.

In one embodiment of the present invention a polynucleotide of the present invention, in particular comprising a cis-regulatory element used herein, is able to induce apomixis in a plant ovule by activating or derepressing, in particular by structurally changing, a regulatory element of an endogenously present gene coding for a protein with an exonuclease activity capable of inducing apomixis in a plant, preferably by expression in the plant ovule. Such a gene is in particular characterised by having a regulatory nucleotide core sequence according to the present invention and thereby allowing, upon derepression, that means induction, the expression of said endogenously coded protein with an exonuclease activity capable of inducing apomixis in the plant.

In the context of the present invention, the term “inducing the expression of a gene—or polynucleotide—coding for protein capable of inducing apomixis” therefore refers to the activation, hereinafter also termed derepression, of a regulatory element governing the expression of said coding sequence, that means refers to the activation of expression allowing the production of a functional apollo protein in the plant ovule.

In a particularly preferred embodiment the biological activity exerted by a polypeptide used in the present invention, that means a protein capable of inducing apomixis in a plant, is a specific exonuclease activity characterised by a specificity in so far as its expression is activated in the ovule of an apomictic plant and repressed or inactivated in a sexual plant.

In particular, the presently used protein, namely the apollo protein, which is capable of inducing apomixis in a plant, in particular a plant ovule and having a specific exonuclease activity is, without being bound by theory, a DEDD 3′→5′ exonuclease, also termed a DNA Q protein, which preferably is characterised by four acidic residues, namely three aspartats (D) and glutamate (E) distributed in three separate sequence segments, namely exo I, exo II and exo III (Moser et al., Nucl. Acids. Res 25 (1997), 5110-5118). Furthermore, these proteins are characterised by either a tyrosine (y) or histidine (h) amino acid located at its active side determinative for being a DEDDy or DEDDh protein. In a preferred embodiment, the present polypeptide capable of inducing apomixis in a plant ovule is a DEDDh exonuclease, preferably comprising the amino acid sequence as given in any one of SEQ ID No. 1 to 3, 10 to 12 or 16 to 18, preferably catalysing the excision of nucleoside monophosphates at the DNA or RNA termini in the 3′-5′ direction. In particular, the present exonuclease is a plant DEDDh exonuclease.

In a particularly preferred embodiment the specific biological activity performed by the polypeptide capable of inducing apomixis in the plant ovule in said plant ovule, that means the apollo protein, appears to be a meiosis-modifying, in particular meiosis-altering, changing or varying activity, in particular is a meiosis-inhibiting activity thereby preventing the reduction of chromosome number in the germ cells.

The isolated and/or used nucleic acid molecules used in the present invention may be present in isolated form. The isolated nucleic acid molecules used in the present invention may, however, also be combined with other nucleic acid molecules, for instance regulatory elements or vectors, thereby forming another molecule comprising not solely the nucleic acid molecule of the present invention. In this case the “nucleic acid molecule “of the present invention is also termed a “nucleic acid sequence” of the present invention.

In the context of the present invention the term “comprising” is understood to have the meaning of “including” or “containing” which means that one first entity contains a second entity, wherein said first entity may in addition to the second entity further contain a third entity. Thus, in particular, the term “a nucleic acid molecule comprising a polynucleotide” means that the nucleic acid molecule of the present invention contains a polynucleotide or a polynucleotide variant of the present invention, but may in addition contain other nucleotides or polynucleotides. In a particular preferred embodiment the term “comprising” as used herein is also understood to mean “consisting of” thereby excluding the presence of other elements besides the explicitly mentioned element. Thus, the present invention also relates to nucleic acid molecules which consist of polynucleotides or polynucleotide variants of the present invention, meaning that the nucleic acid molecule is only composed of the polynucleotide or polynucleotide variant of the present invention and does not comprise any further nucleotides, polynucleotides or other elements. According to this embodiment, the nucleic acid molecule of the present invention is the polynucleotide or polynucleotide variant of the present invention.

Both, the nucleic acid molecule used in the present invention and the polynucleotide comprised therein do exhibit the desired biological activity of being capable of inducing apomixis.

The term “apomixis” refers to the replacement of the normal sexual reproduction by asexual reproduction, that means preferably reproduction without fertilisation of the egg cell, in particular that means only fertilisation of the central cell which is a pseudogamous event, in particular without any fertilisation, in particular the term refers to asexual reproduction through seeds, leading to apomictically produced offsprings or progeny genetically identical to the parent plant, in particular the female plant.

The term “gene” refers to a coding nucleotide sequence and associated regulatory nucleotide sequences. The coding sequence is transcribed into RNA, which depending on the specific gene, will be mRNA, rRNA, tRNA, snRNA, sense RNA or antisense RNA. Examples of regulatory sequences, hereinafter also termed regulatory elements, are promoter sequences, 5′ and 3′ untranslated sequences and termination sequences. Further elements that may be present are, for example, introns or enhancers. A structural gene may constitute an uninterrupted coding region or it may include one or more introns bounded by appropriate splice junctions. The structural gene may be a composite of segments derived from different sources, naturally occurring or synthetic.

The gene to be expressed may be modified in that known mRNA instability motifs or polyadenylation signals are removed or codons which are preferred by the plant into which the sequence is to be inserted may be used.

The present invention also uses the present nucleic acid molecules, in particular a polynucleotide or polynucleotide variant of the present invention, in particular a DNA sequence, wherein said nucleic acid molecule or sequence encodes a polypeptide capable of inducing apomixis, in particular in a plant, preferably plant ovule, and having, preferably comprising, the amino acid sequence depicted in SEQ ID No. 1, 2, 3, 10, 11, 12, 16, 17 or 18, or a polypeptide variant thereof, that means a functional equivalent of a polypeptide used in the present invention, preferably a polypeptide being in terms of biological activity similar thereto. The present invention, thus, also uses a polypeptide variant of the present invention, in particular having a length of at least 150, at least 200, at least 250, at least 300, at least 350, at least 400, at least 450, at least 500 amino acids which after alignment reveals at least 40% and preferably at least 50%, at least 60%, at least 70%, at least 80%, at least 90%, at least 95%, at least 99% or more sequence identity with the, preferably full-length, polypeptide of the present invention, in particular as characterised in any one used in SEQ ID No. 1 to 21, preferably 4, 5, 6, 7, 8, 9, 13, 14, 15, 19, 20 or 21.

The terms “protein” and “polypeptide” are used interchangeably and refer to a molecule with a particular amino acid sequence comprising at least 20, 30, 40, 50 or 60 amino acid residues.

The term “polypeptide” thus means proteins used in the present invention and variants thereof, in particular protein fragments, modified proteins, amino acid sequences and synthetic amino acid sequences. According to the present invention, the polypeptide can be glycosylated or not.

A polypeptide variant used in the present invention which is truncated is also termed a “fragment” used in the present invention. Thus, the term “fragment” refers to a portion of a polynucleotide sequence or a portion of a polypeptide, that means an amino acid sequence of the present invention and hence polypeptide encoded thereby. Fragments of a polynucleotide sequence such as SEQ ID No. 26, 31, 36, 39, 42, 45, 48, 51 or 54, may encode polypeptide fragments that retain the biological activity of the polypeptide of the present invention, such as given in any one of SEQ ID No. 1, 2, 3, 10, 11, 12, 16, 17 or 18. Alternatively, fragments of a polynucleotide sequence that are useful as hybridization probes generally do not encode fragments of a polypeptide retaining biological activity. Fragments of a polynucleotide sequence are generally greater than 20, 30, 50, 100, 150, 200 or 300 nucleotides and up to the entire nucleotide sequence encoding the polypeptide used in the present invention. Generally, the fragments have a length of less than 1000 nucleotides and preferably less than 500 nucleotides. Fragments used in the invention include antisense sequences used to decrease expression of the present polynucleotides. Such antisense fragments may vary in length ranging from at least 20 nucleotides, 50 nucleotides, 100 nucleotides, up to and including the entire coding sequence.

The term “regulatory element” refers to a sequence located upstream (5′), within and/or downstream (3′) to a coding sequence whose transcription and expression is controlled by the regulatory element, potentially in conjunction with the protein biosynthetic apparatus of the cell. “Regulation” or “regulate” refer to the modulation of the gene expression induced by DNA sequence elements located primarily, but not exclusively upstream (5′) from the transcription start of the gene of interest. Regulation may result in an all or none response to a stimulation, or it may result in variations in the level of gene expression. In the context of the present invention a regulatory element is preferably a cis-regulatory element.

A regulatory element, in particular DNA sequence, such as a promoter is said to be “operably linked to” or “associated with” a DNA sequence that codes for a RNA or a protein, if the two sequences are situated and orientated such that the regulatory DNA sequence effects expression of the coding DNA sequence.

A “promoter” is a DNA sequence initiating transcription of an associated DNA sequence, in particular being located upstream (5′) from the start of transcription and being involved in recognition and being of the RNA-polymerase. Depending on the specific promoter region it may also include elements that act as regulators of gene expression such as activators, enhancers, and/or repressors. A regulatory nucleotide core sequence and a regulatory nucleotide target sequence of the present invention is usually part of such a promoter.

A “3′ regulatory element” (or “3′ end”) refers to that portion of a gene comprising a DNA segment, excluding the 5′ sequence which drives the initiation of transcription and the structural portion of the gene, that determines the correct termination site and contains a polyadenylation signal and any other regulatory signals capable of effecting messenger RNA (mRNA) processing or gene expression. The polyadenylation signal is usually characterised by effecting the addition of polyadenylic acid tracts to the 3′ end of the mRNA precursor. Polyadenylation signals are often recognised by the presence of homology to the canonical form 5′-AATAAA-3′.

The term “coding sequence” refers to that portion of a gene encoding a protein, polypeptide, or a portion thereof, and excluding the regulatory sequences which drive the initiation or termination of transcription.

The gene, coding sequence or the regulatory element may be one normally found in the cell, in which case it is called “endogenous” or “autologous”, or it may be one not normally found in a cellular location, in which case it is termed “heterologous”, “exogenous” or “transgenic”.

A “heterologous” gene, coding sequence or regulatory element may also be autologous to the cell but is, however, arranged in an order and/or orientation or in a genomic position or environment not normally found or occurring in the cell in which it is transferred.

The term “vector” refers to a recombinant DNA construct which may be a plasmid, virus, autonomously replicating sequence, an artificial chromosome, such as the bacterial artificial chromosome BAC, phage or other nucleotide sequence, in which at least two nucleotide sequences, at least one of which is a nucleic acid molecule of the present invention, have been joined or recombined. A vector may be linear or circular. A vector may be composed of a single or double stranded DNA or RNA. A vector may be derived from any source. Such a vector is preferably capable of introducing the regulatory element, for instance a promoter fragment, and the nucleic acid molecute of the present invention, preferably a DNA sequence for inducing apomixis, in a plant, in sense or antisense orientation along with appropriate 3′ untranslated sequence into a cell, in particular a plant cell. In the context of the present invention the term “vector” is used interchangeably with the term “plant vector”.

The term “expression” refers to the transcription and/or translation of an endogenous gene or a transgene in plants.

“Marker genes” usually encode a selectable or screenable trait. Thus, expression of a “selectable marker gene” gives the cell a selective advantage which may be due to their ability to grow in the presence of a negative selective agent, such as an antibiotic or a herbicide compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell gives the cell both, a negative and a positive selective advantage. On the other hand a “screenable marker gene” does not confer a selective advantage to a transformed cell, but its expression makes the transformed cell phenotypically distinct from untransformed cells.

The term “expression in the vicinity of the embryo sac” refers to expression in carpel, integuments, ovule, ovule primordium, ovary wall, chalaza, nucellus, funicle or placenta. The term “integuments” refers to tissues which are derived therefrom, such as endothelium. The term “embryogenic” refers to the capability of cells to develop into an embryo under permissive conditions.

The term “plant” refers to any plant, but particularly seed plants.

The term “transgenic plant” or “transgenic plant cell” or “transgenic plant material” refers to a plant, plant cell or plant material which is characterised by the presence of a polynucleotide or polynucleotide variant of the present invention, which may—in case it is autologous to the plant—either be located at another place or in another orientation than usually found in the plant, plant cell or plant material or which is heterologous to the plant, plant cell or plant material. Preferably, the transgenic plant, plant cell or plant material expresses the polynucleotide or its variants such as to induce apomixis.

The term “plant cell” describes the structural and physiological unit of the plant, and comprises a protoplast and a cell wall. The plant cell may be in form of an isolated single cell, such as a stomatal guard cells or a cultured cell, or as a part of a higher organized unit such as, for example, a plant tissue, or a plant organ.

The term “plant material” includes plant parts, in particular plant cells, plant tissue, in particular plant propagation material, preferably leaves, stems, roots, emerged radicles, flowers or flower parts, petals, fruits, pollen, pollen tubes, anther filaments, ovules, embryo sacs, egg cells, ovaries, zygotes, embryos, zygotic embryos per se, somatic embryos, hypocotyl sections, apical meristems, vascular bundles, pericycles, seeds, roots, cuttings, cell or tissue cultures, or any other part or product of a plant.

Thus, the present invention also provides plant propagation material of the transgenic plants of the present invention. Said “plant propagation material” is understood to be any plant material that may be propagated sexually or asexually in vivo or in vitro. Particularly preferred within the scope of the present invention are protoplasts, cells, calli, tissues, organs, seeds, embryos, pollen, egg cells, zygotes, together with any other propagating material obtained from transgenic plants. Parts of plants, such as for example flowers, stems, fruits, leaves, roots originating in transgenic plants or their progeny previously transformed by means of the methods of the present invention and therefore consisting at least in part of transgenic cells, are also an object of the present invention. Especially preferred plant materials, in particular plant propagation materials, are apomictic seeds.

Particularly preferred plants are monocotyledonous or dicotyledonous plants. Particularly preferred are crop or agricultural plants, such as sunflower, peanut, corn, potato, sweet potato, bean, pea, chicory, lettuce, endive, cabbage, cauliflower, broccoli, turnip, radish, spinach, onion, garlic, eggplant, celery, carrot, squash, pumpkin, zucchini, cucumber, apple, pear, melon, strawberry, grape, raspberry, pineapple, soybean, Cannabis, Humulus (hop), tomato, sorghum, sugar cane, and non-fruit bearing trees such as poplar, rubber, Paulownia, pine, elm,Lolium, Festuca, Dactylis, alfalfa, safflower, tobacco, cassava, coffee, coconut, pineapple, citrus trees, cocoa, tea, banana, avocado, fig, guava, mango, olive, papaya, cashew, macadamia, almond, green beans, lima beans, peas, fir, hemlock, spruce, redwood, in particular maize, wheat, barley, sorghum, rye, oats, turf and forage grasses, millet, rice and sugar cane. Especially preferred are maize, wheat, sorghum, rye, oats, turf grasses and rice.

Particularly preferred are also ornamental plants such as ornamental flowers and ornamental crops, for instance Begonia, Carnation, Chrysanthemum, Dahlia, Gardenia, Asparagus, Geranium, Daisy, Gladiolus, Petunia, Gypsophila, Lilium, Hyacinth, Orchid, Rose, Tulip, Aphelandra, Aspidistra, Aralia, Clivia, Coleus, Cordyline, Cyclamen, Dracaena, Dieffnbachia, Ficus, Philodendron, Poinsettia, Fern, Ivy, Hydrangea, Limonium, Monstera, Palm, Date-palm, Potho, Singonio, Violet, Daffodil, Lavender, Lily, Narcissus, Crocus, Iris, Peonies, Zephyranthes, Anthurium, Gloxinia, Azalea, Ageratum, Bamboo, Camellia, Dianthus, Impatien, Lobelia, Pelargonium, Lilac, Lily of the Valley,Stephanotis, Hydrangea, Sunflower, Gerber daisy, Oxalis, Marigold and Hibiscus.

Among the dicotyledonous plants Arabidopsis, Boechera, soybean, cotton, sugar beet, oilseed rape, tobacco, pepper, melon, lettuce, Brassica vegetables, in particular Brassica napus, sugar beet, oilseed rape and sunflower are more preferred herein.

“Transformation”, “transforming” and “transferring” refers to methods to transfer nucleic acid molecules, in particular DNA, into cells including, but not limited to, biolistic approaches such as particle bombardment, microinjection, permeabilising the cell membrane with various physical, for instance electroporation, or chemical treatments, for instance polyethylene glycol or PEG, treatments; the fusion of protoplasts or Agrobacterium tumefaciens or rhizogenes mediated trans-formation. For the injection and electroporation of DNA in plant cells there are no specific requirements for the plasmids used. Plasmids such as pUC derivatives can be used. If whole plants are to be regenerated from such transformed cells, the use of a selectable marker is preferred. Depending upon the method for the introduction of desired genes into the plant cell, further DNA sequences may be necessary; if, for example, the Ti or Ri plasmid is used for the transformation of the plant cell, at least the right border, often, however, the right and left border of the Ti and Ri plasmid T-DNA have to be linked as flanking region to the genes to be introduced. Preferably, the transferred nucleic acid molecules are stably integrated in the genome or plastome of the recipient plant.

In the context of the present invention it is understood that “transforming” a plant cell refers to the transformation process itself and the subsequent stable integration of the transgenic, that means exogenous, nucleotide sequence in the genome of the plant cell.

The expression “progeny” or “offspring” refers to both, “asexually” and “sexually” generated progeny of transgenic plants. This definition is also meant to include all mutants and variants obtainable by means of known processes, such as for example cell fusion or mutant selection and which still exhibit the characteristic properties of the initial transformed plant of the present invention, together with all crossing and fusion products of the transformed plant material. This also includes progeny plants that result from a backcrossing, as long as the said progeny plants still contain the polynucleotide and/or polypeptide according to the present invention.

The isolated nucleic acid molecule used in the present invention is preferably a DNA, preferably a DNA from a plant, preferably from Brassicaceae, in particular Boechera, in particular Boechera holboellii, Boechera divaricarpa or Boechera stricta, in a particular genomic or cDNA sequence molecule. It may, however, also be a RNA, in particular mRNA.

The present invention also uses in a preferred embodiment a plant vector comprising any one of the nucleic acid sequences according to the present invention. Both, the specific polynucleotide or the polynucleotide variant used in the present invention can be contained in the vector in sense or antisense orientation to a regulatory element.

In a preferred embodiment of the present invention the plant vector comprises a polynucleotide, in particular the cis-acting regulatory element of the present invention capable of acting as a regulatory element operably linked to a protein coding nucleic acid sequence desired to be expressed in a plant, in particular a plant ovule.

The present invention also uses in a preferred embodiment a host cell containing the vector of the present invention.

The present invention also provides a transgenic plant, plant cell, plant material, in particular plant seed comprising at least one nucleic acid molecule according to the present invention or the vector of the present invention. The present invention also provides in a preferred embodiment a cell culture, preferably a plant cell culture comprising a cell according to the present invention.

In a particularly preferred embodiment the present invention provides a transgenic plant, plant cell, plant material, in particular plant seed, wherein the polynucleotide, the polypeptide or the variant thereof exhibit its biological function. In a particular embodiment of the present invention a plant or plant seed is provided which comprises the polynucleotide, polypeptide or variants thereof of the present invention and which show due to the presence of said polynucleotide or polypeptide or variant thereof apomixis.

Whilst the present invention is particularly described by way of the production of apomictic seed by heterologous expression of a polynucleotide of the present invention, it will be recognized that variants of the present polynucleotides, the products of which have a similar structure and function may likewise be expressed with similar results. Moreover, although the example illustrates apomictic seed production in Boechera and Arabidopsis, the invention is, of course, not limited to the expression of apomictic seed-inducing genes solely in these plants.

Further preferred embodiments of the present invention are the subject matter of the subclaims.

The figures show:

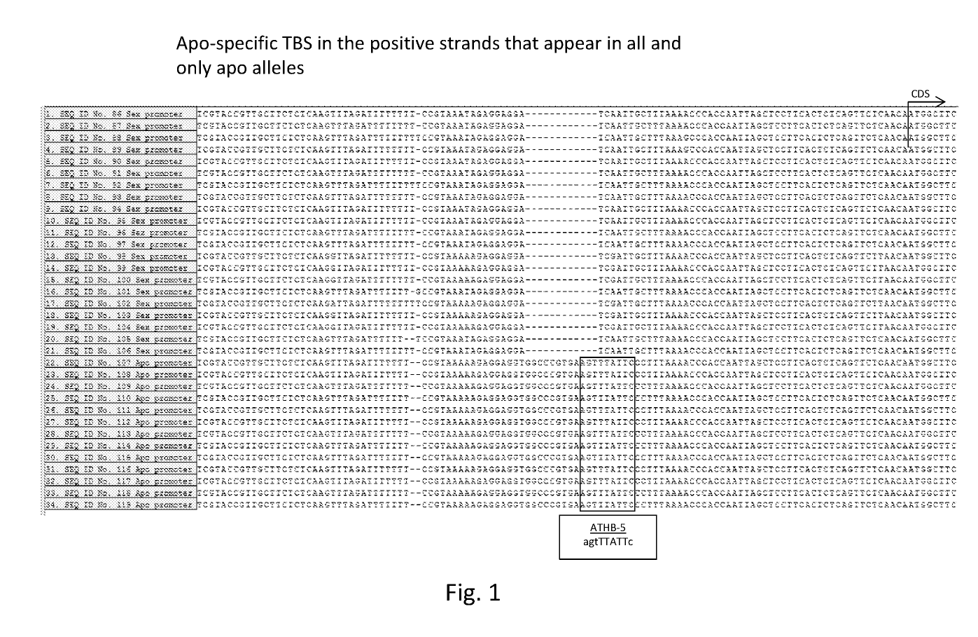

FIG. 1 apo-specific TBS in the positive strands that appear in all and only apo alleles. (SEQ ID NO.: 86, nucleotide residues 1-112; SEQ ID NO.: 87, nucleotide residues 1-112; SEQ ID NO.: 88, nucleotide residues 1-113; SEQ ID NO.: 89, nucleotide residues 1-112; SEQ ID NO.: 90, nucleotide residues 1-112; SEQ ID NO.: 91, nucleotide residues 1-112; SEQ ID NO.: 92, nucleotide residues 1-113; SEQ ID NO.: 93, nucleotide residues 1-112; SEQ ID NO.: 94, nucleotide residues 1-112; SEQ ID NO.: 95, nucleotide residues 1-112; SEQ ID NO.: 96, nucleotide residues 1-112; SEQ ID NO.: 97, nucleotide residues 1-112; SEQ ID NO.: 98, nucleotide residues 1-112; SEQ ID NO.: 99, nucleotide residues 1-112; SEQ ID NO.: 100, nucleotide residues 1-112; SEQ ID NO.: 101, nucleotide residues 1-112; SEQ ID NO.: 102, nucleotide residues 1-113; SEQ ID NO.: 103, nucleotide residues 1-112; SEQ ID NO.: 104, nucleotide residues 1-112; SEQ ID NO.: 105, nucleotide residues 1-111; SEQ ID NO.: 106, nucleotide residues 1-112; SEQ ID NO.: 107, nucleotide residues 1-123; SEQ ID NO.: 108, nucleotide residues 1-123; SEQ ID NO.: 109, nucleotide residues 1-123; SEQ ID NO.: 110, nucleotide residues 1-123; SEQ ID NO.: 111, nucleotide residues 1-123; SEQ ID NO.: 112, nucleotide residues 1-123; SEQ ID NO.: 113, nucleotide residues 1-123; SEQ ID NO.: 114, nucleotide residues 1-123; SEQ ID NO.: 115, nucleotide residues 1-123; SEQ ID NO.: 116, nucleotide residues 1-123; SEQ ID NO.: 117, nucleotide residues 1-123; SEQ ID NO.: 118, nucleotide residues 1-123; SEQ ID NO.: 119, nucleotide residues 1-123; ATHB-5 (agtTTATTc), nucleotide residues 62-70 of SEQ ID NO.: 107).

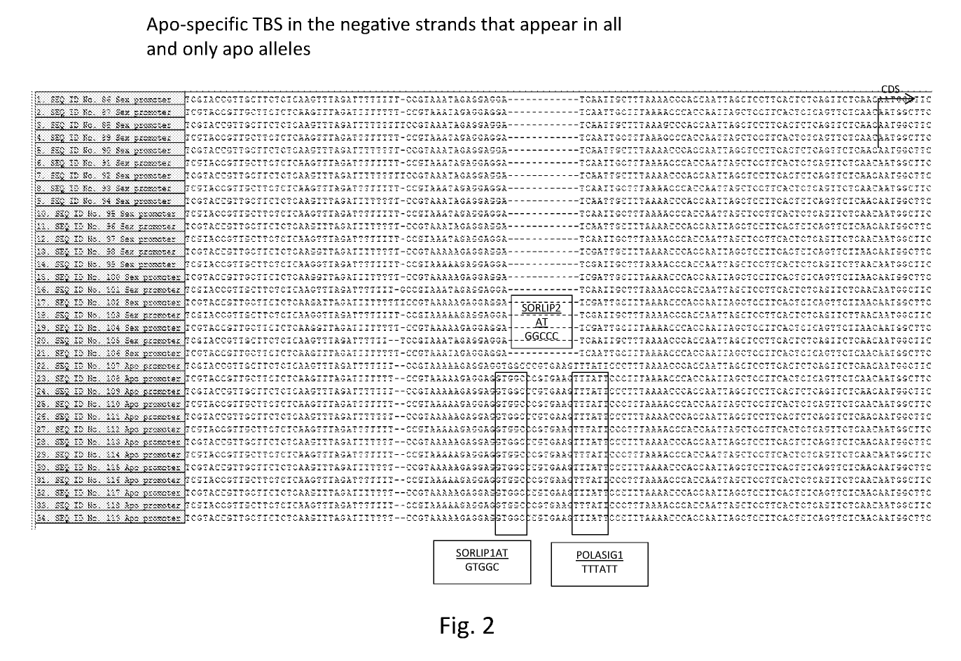

FIG. 2 apo-specific TBS in the negative strands that appear in all and only apo alleles. (SEQ ID NO.: 86, nucleotide residues 1-112; SEQ ID NO.: 87, nucleotide residues 1-112; SEQ ID NO.: 88, nucleotide residues 1-113; SEQ ID NO.: 89, nucleotide residues 1-112; SEQ ID NO.: 90, nucleotide residues 1-112; SEQ ID NO.: 91, nucleotide residues 1-112; SEQ ID NO.: 92, nucleotide residues 1-113; SEQ ID NO.: 93, nucleotide residues 1-112; SEQ ID NO.: 94, nucleotide residues 1-112; SEQ ID NO.: 95, nucleotide residues 1-112; SEQ ID NO.: 96, nucleotide residues 1-112; SEQ ID NO.: 97, nucleotide residues 1-112; SEQ ID NO.: 98, nucleotide residues 1-112; SEQ ID NO.: 99, nucleotide residues 1-112; SEQ ID NO.: 100, nucleotide residues 1-112; SEQ ID NO.: 101, nucleotide residues 1-112; SEQ ID NO.: 102, nucleotide residues 1-113; SEQ ID NO.: 103, nucleotide residues 1-112; SEQ ID NO.: 104, nucleotide residues 1-112; SEQ ID NO.: 105, nucleotide residues 1-111; SEQ ID NO.: 106, nucleotide residues 1-112; SEQ ID NO.: 107, nucleotide residues 1-123; SEQ ID NO.: 108, nucleotide residues 1-123; SEQ ID NO.: 109, nucleotide residues 1-123; SEQ ID NO.: 110, nucleotide residues 1-123; SEQ ID NO.: 111, nucleotide residues 1-123; SEQ ID NO.: 112, nucleotide residues 1-123; SEQ ID NO.: 113, nucleotide residues 1-123; SEQ ID NO.: 114, nucleotide residues 1-123; SEQ ID NO.: 115, nucleotide residues 1-123; SEQ ID NO.: 116, nucleotide residues 1-123; SEQ ID NO.: 117, nucleotide residues 1-123; SEQ ID NO.: 118, nucleotide residues 1-123; SEQ ID NO.: 119, nucleotide residues 1-123; SORLIP1AT (GTGGC), nucleotide residues 51-55 of SEQ ID NO.: 107; SORLIP2AT (GGCCC), nucleotide residues 53-57 of SEQ ID NO.: 107; POLASIG1 (TTTATT), nucleotide residues 64-69 of SEQ ID NO.: 107)

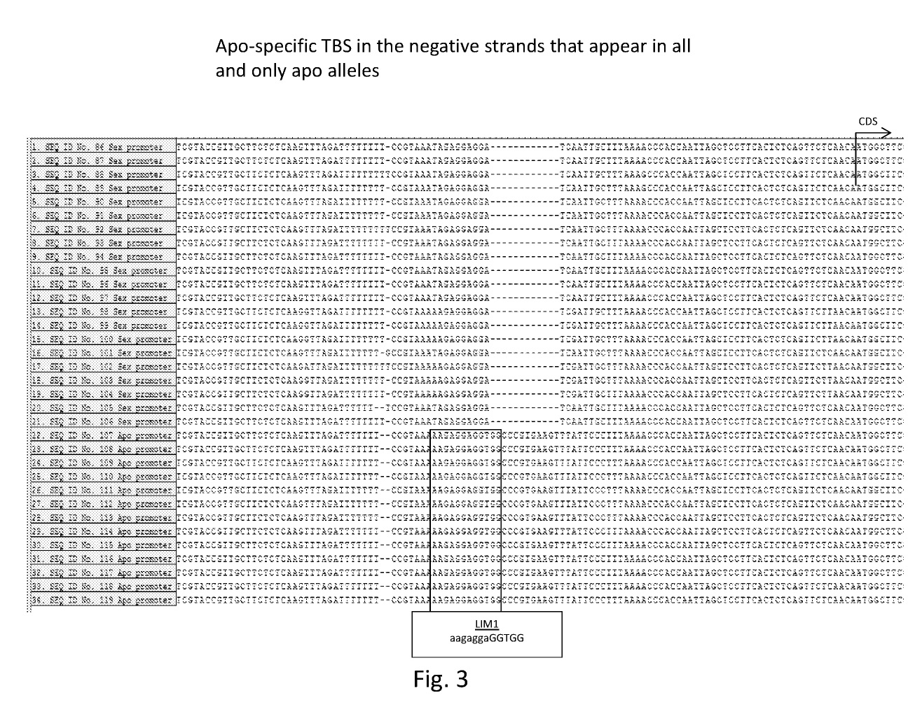

FIG. 3 apo-specific TBS in the negative strands that appear in all and only apo alleles. (SEQ ID NO.: 86, nucleotide residues 1-112; SEQ ID NO.: 87, nucleotide residues 1-112; SEQ ID NO.: 88, nucleotide residues 1-113; SEQ ID NO.: 89, nucleotide residues 1-112; SEQ ID NO.: 90, nucleotide residues 1-112; SEQ ID NO.: 91, nucleotide residues 1-112; SEQ ID NO.: 92, nucleotide residues 1-113; SEQ ID NO.: 93, nucleotide residues 1-112; SEQ ID NO.: 94, nucleotide residues 1-112; SEQ ID NO.: 95, nucleotide residues 1-112; SEQ ID NO.: 96, nucleotide residues 1-112; SEQ ID NO.: 97, nucleotide residues 1-112; SEQ ID NO.: 98, nucleotide residues 1-112; SEQ ID NO.: 99, nucleotide residues 1-112; SEQ ID NO.: 100, nucleotide residues 1-112; SEQ ID NO.: 101, nucleotide residues 1-112; SEQ ID NO.: 102, nucleotide residues 1-113; SEQ ID NO.: 103, nucleotide residues 1-112; SEQ ID NO.: 104, nucleotide residues 1-112; SEQ ID NO.: 105, nucleotide residues 1-111; SEQ ID NO.: 106, nucleotide residues 1-112; SEQ ID NO.: 107, nucleotide residues 1-123; SEQ ID NO.: 108, nucleotide residues 1-123; SEQ ID NO.: 109, nucleotide residues 1-123; SEQ ID NO.: 110, nucleotide residues 1-123; SEQ ID NO.: 111, nucleotide residues 1-123; SEQ ID NO.: 112, nucleotide residues 1-123; SEQ ID NO.: 113, nucleotide residues 1-123; SEQ ID NO.: 114, nucleotide residues 1-123; SEQ ID NO.: 115, nucleotide residues 1-123; SEQ ID NO.: 116, nucleotide residues 1-123; SEQ ID NO.: 117, nucleotide residues 1-123; SEQ ID NO.: 118, nucleotide residues 1-123; SEQ ID NO.: 119, nucleotide residues 1-123; LIM1 (aagaggaGGTGG), nucleotide residues 43-54 of SEQ ID NO.: 107).

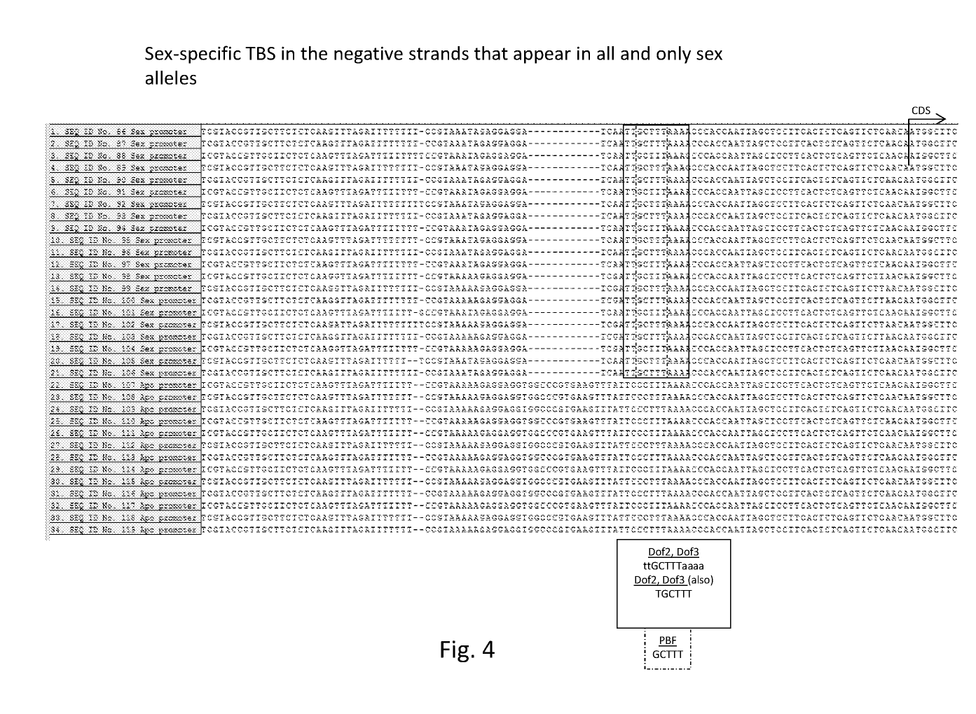

FIG. 4 sex-specific TBS in the negative strands that appear in all and only sex alleles._(SEQ ID NO.: 86, nucleotide residues 1-112; SEQ ID NO.: 87, nucleotide residues 1-112; SEQ ID NO.: 88, nucleotide residues 1-113; SEQ ID NO.: 89, nucleotide residues 1-112; SEQ ID NO.: 90, nucleotide residues 1-112; SEQ ID NO.: 91, nucleotide residues 1-112; SEQ ID NO.: 92, nucleotide residues 1-113; SEQ ID NO.: 93, nucleotide residues 1-112; SEQ ID NO.: 94, nucleotide residues 1-112; SEQ ID NO.: 95, nucleotide residues 1-112; SEQ ID NO.: 96, nucleotide residues 1-112; SEQ ID NO.: 97, nucleotide residues 1-112; SEQ ID NO.: 98, nucleotide residues 1-112; SEQ ID NO.: 99, nucleotide residues 1-112; SEQ ID NO.: 100, nucleotide residues 1-112; SEQ ID NO.: 101, nucleotide residues 1-112; SEQ ID NO.: 102, nucleotide residues 1-113; SEQ ID NO.: 103, nucleotide residues 1-112; SEQ ID NO.: 104, nucleotide residues 1-112; SEQ ID NO.: 105, nucleotide residues 1-111; SEQ ID NO.: 106, nucleotide residues 1-112; SEQ ID NO.: 107, nucleotide residues 1-123; SEQ ID NO.: 108, nucleotide residues 1-123; SEQ ID NO.: 109, nucleotide residues 1-123; SEQ ID NO.: 110, nucleotide residues 1-123; SEQ ID NO.: 111, nucleotide residues 1-123; SEQ ID NO.: 112, nucleotide residues 1-123; SEQ ID NO.: 113, nucleotide residues 1-123; SEQ ID NO.: 114, nucleotide residues 1-123; SEQ ID NO.: 115, nucleotide residues 1-123; SEQ ID NO.: 116, nucleotide residues 1-123; SEQ ID NO.: 117, nucleotide residues 1-123; SEQ ID NO.: 118, nucleotide residues 1-123; SEQ ID NO.: 119, nucleotide residues 1-123; Dof2/Dof3 (ttGCTTTaaaa), nucleotide residues 58-68 of SEQ ID NO.: 86; Dof2/Dof3 (also) (TGCTTT), nucleotide residues 59-64 of SEQ ID NO.: 86; PBF (GCTTT), nucleotide residues 60-64 of SEQ ID NO.: 86).

The invention will now be illustrated by way of example.

### EXAMPLE 1

### Screening and Isolation of Apomixis-Inducing Gene (Apollo Gene)

1.a) Plant Material and Seed Screen Analysis

Plants were grown from seedlings onwards in a phytotron under controlled environmental conditions. The flow cytometric seed screen was used to analyse reproductive variability in 18 Boechera accessions (Table IV).

TABLE IV
Boechera accessions used in
Microarrays and RT-PCR analyses.
Table IV - Boechera accessions used in
Microarrays and RT-PCR analyses.

| | Apomeiosis | |
|---|---|---|
| Accession | frequency | Collection locality |
| B08-1 | 1 | Birch Creek, Montana |
| B08-11 | 1 | Sliderock, Ranch Creek, Granite, Montana |
| B08-33 | 1 | Mule Ranch, Montana |
| B08-111 | 1 | Morgan Switch Back, Idaho |
| B08-81 | 1 | Vipond Park, Beaverhead, Montana |
| B08-168 | 1 | Vipond Park, Beaverhead, Montana |
| B08-43 | 1 | Mule Ranch, Montana |
| B08-66 | 1 | Highwood Mtns, Montana |
| B08-104 | 1 | Lost Trail Meadow |
| B08-215 | 1 | Blue Lakes road, California |
| B08-369 | 0 | Twin Saddle, Idaho |
| B08-376 | 0 | Sagebrush Meadow, Montana |
| B08-380 | 0 | Buffalo Pass, Colorado |
| B08-355 | 0 | Gold Creek, Colorado |
| B08-329 | 0 | Big Hole Pass, Montana |
| B08-385 | 0 | Parker Meadow, Idaho |
| B08-344 | 0 | Bandy Ranch, Montana |
| B08-390 | 0 | Panther Creek |

Single seeds were ground individually with three 2.3 mm stainless steel beads in each well of 96-well plate (PP-Master-block 128.0/85 MM, 1.0 ml 96 well plate by Greiner bio-one, http World Wide Web internet site “gbo.com”) containing 50 μl extraction-nuclei isolation buffer (see below) using a Geno-Grinder 2000 (SPEX Certi-Prep) at rate of 150 strokes/minute for 90 seconds.

A two-step procedure consisting of an isolation and staining buffer was used: (a) isolation buffer I—0.1M Citric acid monohydrate and 0.5% v/v Tween 20 dissolved in H₂O and adjusted to pH 2.5); and (b) staining buffer II—0.4M Na₂HPO₄.12H₂O dissolved in H₂O plus 4 μg/ml 4′,6-Diamidinophenyl-indole (DAPI) and adjusted to pH 8.5. 50 μl of isolation buffer I was added to each seed per well in a 96-well plate before grinding, and a further 160 μl buffer I was added after grinding to recover enough volume through filtration (using Partec 30 μm mesh-width nylon filters). 100 μl of staining buffer II was then added to 50 μl of the resultant suspension (isolated nuclei), and incubated on ice for 10 minutes before flow cytometric analysis. To avoid sample degradation over the 2-hour period required for the analysis of 96 samples, the sample plate was sealed with aluminum sealing tape.

All sample plates were analysed on a 4° C. cooled Robby-Well auto-sampler hooked up to a Partec PAII flow Cytometer (Partec GmbH, Münster, Germany). Two single seeds from SAD 12, a known sexual self-fertile Boechera were always included as an external reference at well positions 1 and 96 in order to normalize other peaks and correct peak shifts over the analysis period. SAD 12 seeds were composed exclusively of 2C embryo to 3C endosperm ratio, which reflected an embryo composition of C (C denotes monoploid DNA content) maternal (Cm) genomes+C paternal (Cp)=2C genomes, and an endosperm composition of 2Cm+Cp=3C.

Based upon the present high-throughput flow-cytometric seed screen data, all apomictic accessions were shown to be characterized by 100% apomictic seed production.

1.b) Ovule Micro-Dissection

Ovules at megasporogenesis between stages 2-II to 2-IV were selected where megaspore mother cell is differentiated, inner and outer integument initiated in order to examine changes in gene expression associated with meiosis and apomeiosis. The gynoecia of sexual and apomictic Boechera were dissected out from non-pollinated flowers at the stage of megasporogenesis in 0.55 M sterile mannitol solution, at a standardized time (between 8 and 9 a.m.) over multiple days. Microdissections were done in a sterile laminar air flow cabinet using a stereoscopic Microscope (1000 Stemi, Carl Zeiss, Jena, Germany) under 2× magnification. The gynoecium was held with forceps while a sterile scalpel was used to cut longitudinally such that the halves of the silique along with the ovules were immediately exposed to the mannitol. Individual live ovules were subsequently collected under an inverted Microscope (Axiovert 200M, Carl Zeiss) in sterile conditions, using sterile glass needles (self-made using a Narishige PC-10 puller, and bent to an angle of about)100° to isolate the ovules from placental tissue. Using a glass capillary (with an opening of 150 μm interior diameter) interfaced to an Eppendorf Cell Tram Vario, the ovules were collected in sterile Eppendorf tubes containing 100 μl of RNA stabilizing buffer (RNA later, Sigma). Between 20 and 40 ovules per accession were collected in this way, frozen directly in liquid nitrogen and stored at −80° C.

1.c) Ovule RNA Isolation

Total RNA extractions were carried out using PicoPure RNA isolation kit (Arcturus Bioscience, CA). RNA integrity and quantity was verified on an Agilent 2100 Bioanalyzer using the RNA Pico chips (Agilent Technologies, Palo Alto, Calif.).

1.d) Microarray

1.d.i) Microarray Design

The 454 (FLX) technology was used to sequence the complete transcriptomes of 3 sexual and 3 apomictic Boechera accessions, as a first step in the design of high-density Boechera-specific microarrays for use in comparisons of gene expression and copy number variation. The goal of transcriptome sequencing was thus to identify all genes which can be expressed during flower development, followed by the spotting of all identified genes onto an (Agilent) microarray.

This was accomplished by pooling flowers at multiple developmental stages separately for sexual and apomictic plants, followed by a cDNA normalization procedure in order to balance out transcript levels to increase the chance that all observable mRNA species are sequenced. Furthermore, a 3′-UTR (untranslated region) anchored 454 procedure was employed such that mRNA sequences were biased towards their 3′-UTRs, regions which demonstrate relatively high (but not random) levels of variability, to enable the identification of allelic variation.

The 454 sequences were assembled using the CLC Genomics workbench using standard assembly parameters for long-read high-throughput sequences, after trimming of all reads using internal sequence quality scores. In doing so, 36 289 contig sequences and 154 468 non-assembled singleton sequences were obtained. This data was provided to ImaGenes (GmbH, Germany) for microarray development using their Pre-selection strategy (PSS) service.

The PSS service worked as follows: 14 different oligonucleotides (each 60 bp in length) per contig and 8 oligonucleotides per singleton, including the “anti-sense” sequence of each oligo, were bioin-formatically designed and spotted onto two 1 million-spot test arrays. These test-arrays were probed using (1) a “complex cRNA mixture” (obtained by pooling tissues and harvesting all RNA from them), and (2) genomic DNA extracted from leaf tissue pooled from a sexual and an apomictic individual. Based upon the separate hybridization results from the cRNA and genomic DNA samples, and after all quality tests, a final 2×105 000 spot array was designed. This array should contain multiple oligonucleotides (i.e. technical replicates) of every gene expressed during Boechera flower development.

1.d.ii) Hybridization

cRNA was prepared and labelled using the Quick-Amp One-Color Labeling Kit (Agilent Technologies, CA) and hybridized to the Agilent custom Boechera arrays (8 and 10 biological replicates were hybridized for sexual and apomictic genotypes respectively).

1.d.iii) Statistical Analysis

Analyses were performed using GeneSpring GX Software (version 10) and candidate probes significantly differentially expressed (p≤0.05) between apomictic and sexual plants were selected based on the following parameters: (a) percentile shift 75 normalization, median as baseline, reproductive mode (apomictic or sexual) as interpretation (1st level), T-test unpaired as statistical analysis and Bonferroni FWER multiple test corrections. Using the highest level of significance cutoff led to the identification of 4 different spots on the microarray (p<0.01 for the first three and p<0.05 for the fourth). Importantly, when the oligonucleotide sequences of these 4 spots were BLASTed to a 454 cDNA sequence database, all 4 blasted to the same Boechera transcript. Thus, not only has the present experiment been corrected for biological noise, furthermore a single differentially-expressed transcript between the microdissected ovules of all sexual and apomictic genotypes, with 4 technical replicates for the specific gene on the microarray was detected. This gene is expressed to a similar fashion when comparing both diploid and triploid apomictic ovules to those of sexuals, and hence its expression behavior is apparently not influenced by ploidy. Finally, a search for homologues to this Boechera transcript demonstrated that it is involved with the cell cycle in other species, thus supporting evidence regarding deregulation of the sexual pathway as a means to produce apomixis.

### EXAMPLE 2

### Characterisation of Apomixis-Inducing Gene

2.a) Candidate Gene Characterization

2.a.i) Genome Level

2.a.i.1) Cloning

The full-length transcript from all 18 accessions was cloned and sequenced (TOPO-TA Cloning kit, Invitrogen) using proofreading polymerase (Accuprime). The transcript is highly polymorphic, and is characterized by comparable levels of single nucleotide polymorphisms between sexual and apomicts. Nevertheless, a single “apomixis polymorphism” is found in all 10 apomictic accessions, but not in any sexual accession. SEQ ID No. 46 to 54 show the genomic and the coding sequence of three sexual alleles, namely S011a, S355a and S390a. SEQ ID No. 37 to 45 show the genomic and the coding sequence of three apomictic alleles, namely A011a, A043a and A081a. Considering that the geographic collection points of all accessions range from California to the American mid-west (i.e. 1000's of kilometers), the sharing of this polymorphism in all apomicts is highly significant. Finally, the SNP polymorphism spectrum surrounding the “apomixis polymorphism” reflects that found in all other alleles in both sexual and apomictic accessions. Hence the “apomixis polymorphism” appears to have undergone recombination during the evolution of Boechera, but which is nonetheless shared by all apomicts, regardless of different genetic, ploidy or geographic backgrounds.

2.a.i.2) BAC

Pooled DNA of all tissues accessions was used as a template for hybridization probes generation. Two probes of different size (1.6 and 2.3 kb) were prepared by PCR amplification using two pairs of specific primers of the candidate gene genomic sequence. Both probes were labeled and used for hybridization on a apomictic Boechera BAC library. There were 8 positive hybridizations. The respective isolated BACs (PureLink Plasmid DNA Purification kit) were named 1, 2a, 2b, 3, 4, 5, 6 and 7. Selected BACs were retested using specific primers for the candidate gene. All BACs were confirmed except the BAC-3. The other seven BACs were fingerprinted by restriction enzyme digestion. BAC-1 and BAC-2a seemed to be redundant with the other BACs. The BACs: 2b, 4, 5, 6 and 7 were sequenced.

BAC sequences could be assembled together for the pairs 2b_4 and 5_7, whereas BAC-6 remained alone.

BAC sequences were characterized by comparison with other plant sequences.

2.a.ii) Transcriptome Level

RACE experiments (SMARTer RACE cDNA Amplification Kit) were performed.

The results revealed that mRNA corresponding to apomictic accessions has a truncated 5′ extreme upstream the “apomixis polymorphism” whereas sexual accessions have ˜200 bp of additional length.

Once 5′ and 3′ mRNA extremes were known, further PCRs over all tissues cDNA were performed for complete splicing profile characterization.

2.b) Validation

2.b.i) QRT-PCR

An allele-specific qRT-PCR analysis of the candidate gene on the microdissected live ovules (megaspore mother cell stage) from 6 sexual and 10 diploid apomictic Boechera accessions (3 technical replicates per accession) was completed. Using two different forward PCR primers which spanned the apomixis-specific polymorphism which was identified from the gene sequences, it was possible to measure transcript abundance for both the sexual and apomictic alleles separately.

cDNA was prepared using RevertAid H Minus reverse transcriptase.

For the real-time PCR reactions the SYBR® Green PCR Master Mix (Applied Biosystems, Foster City, Calif.) was used. QRT-PCR amplifications were carried out in a 7900HT Fast RT-PCR System machine (Applied Biosystems) with the following temperature profile for SYBRgreen assays: initial denaturation at 90° C. for 10 min, followed by 40 cycles of 95° C. for 15 sec. and 60° C. for 1 min. For checking amplicon quality, a melting curve gradient was obtained from the product at the end of the amplification. The Ct, defined as the PCR cycle at which a statistically significant increase of reporter fluorescence is first detected, was used as a measure for the starting copy numbers of the target gene. The mean expression level and standard deviation for each set of three technical replicates for each cDNA was calculated. Relative quantitation and normalization of the amplifled targets were performed by the comparative AACt method using a calibrator sample in reference to the expression levels of the housekeeping gene UBQ10.

The results are conclusive: the apomictic allele is exclusively expressed in the microdissected ovules of all apomictic accessions, while the sexual allele is never expressed in any, which means sexual or apomictic, ovule. Both alleles are expressed in other tissues, namely somatic tissue. Hence, it appears very reasonable to assume that the sexual allele is inactive/silenced during normal sexual ovule development, while the expression of the apomictic allele is correlated with apomeiotic ovule development.

### EXAMPLE 3

### Transformation of Arabidopsis thaliana with Apomixis-Inducing Gene

3.a) Plant Transformation

Transformations of Arabidopsis thaliana (sex) (hybrids F1) and Boechera (sex) with the gene of the present invention are able to show a change of their reproductive mode into apomictic seed production. For this, the complete genomic allele (including complete promoter) has been cloned in pNOS-ABM.

In addition, different constructs are used to characterize the role of the present regulatory elements, in particular the promoter of the present invention, in its expression. For this, both apo and sex promoters have been exactly connected to the ATG in front of gus in pGUS-ABM.

Complete BAC-4 is as well used for transformations.

### EXAMPLE 4

For promoter analysis of the present regulatory elements the plant PAN software (release 1.0.2007) (http World Wide Web internet site “plantpan.mbc.nctu.edu.tw/gene_group/index.php”; Chang et al., (2008) “PlantPAN: Plant Promoter Analysis Navigator, for identifying combinatorial cis-regulatory elements with distance constraint in plant gene group”, BMC Genomics, 9:561) has been used. What is claimed is:

1-15. (canceled)

16. A method for the production of a transgenic apomictic plant, comprising the following steps:
m) providing a plant cell of a sexually propagating plant, which comprises a nucleotide sequence coding for a protein with DEDDh exonuclease activity under control of a cis-acting regulatory element,
n) modifying the cis-acting regulatory element controlling the nucleotide sequence coding for a protein with DEDDh exonuclease activity by creating at least one ATHB-5 and at least one SORLIP2AT transcription factor binding site therein, and
o) regenerating the plant cell obtained in step n), which contains the newly created at least one regulatory nucleotide core sequence into a transgenic plant exhibiting apomixis.

17. The method according to claim 16, wherein the plant cell provided in step m) is transformed with a plant vector containing an exogenous nucleotide sequence element comprising a nucleotide sequence encoding a trans-acting apomixis effector.

18. The method according to claim 17, wherein the trans-acting apomixis effector is an over expressed trans acting apomixis effector.

19. The method according to claim 18, wherein the trans-acting apomixis effector is a transcription factor, in particular ATHB-5, SORLIP2AT, or POLASIG.

20. The method according to claim 16, wherein the nucleotide sequence coding for a protein with the activity of a DEDDh exonuclease comprises a nucleotide sequence selected from the group consisting of a1) the polynucleotide defined in any one of SEQ ID NO: 22 to 54, or a fully complementary strand thereof, b1) a polynucleotide encoding a polypeptide with the amino acid sequence defined in any one of SEQ ID NO: 1 to 21 or a fully complementary strand thereof and c1) a polynucleotide variant having a degree of sequence identity of more than 70% to the nucleic acid sequence defined in a1) or b1) of a fully complementary strand thereof.

21. The method according to claim 16, wherein the nucleotide sequence coding for a protein with the activity of a DEDDh exonuclease comprises a nucleotide sequence selected from the group consisting of a2) the polynucleotide defined in any one of SEQ ID NO: 22, 23, 27, 28, 32, 33 or a fully complementary strand thereof, b2) a polynucleotide encoding a polypeptide with the amino acid sequence defined in any one of SEQ ID NO: 4, 5, 6 or a fully complementary strand thereof, and c2) a polynucleotide variant having a degree of sequence identity of more than 70% to the nucleic acid sequence defined in a2) or b2) or a fully complementary strand thereof.

22. A transgenic apomictic plant produced according to the method of claim 16.

23. A transgenic plant material from a plant according to claim 22.

24. The method of claim 16, wherein the ATHB-5 binding site is any one of SEQ ID NO: 66 or 67 and the SORLIP2AT binding site is any one of SEQ ID NO: 76 or 77.

25. The method of claim 16, wherein the method further comprises interrupting or deleting at least one regulatory nucleotide target sequence in said cis-acting regulatory element that is a Dof2, a Dof3, or a PBF transcription factor binding site.

26. The method of claim 25, wherein the Dof2, Dof3 or PBF transcription factor binding site is selected from the group consisting of SEQ ID NO: 80, 81, 82, 83, 84, and 85.


SEQUENCE LISTING (119 sequences)

SEQ ID NO: 1 - protein, 165 aa, Boechera
  misc_feature (10)..(10): Xaa can be any naturally occurring amino acid
  misc_feature (12)..(12): Xaa can be any naturally occurring amino acid
  misc_feature (17)..(17): Xaa can be any naturally occurring amino acid
  misc_feature (33)..(33): Xaa can be any naturally occurring amino acid
  misc_feature (39)..(39): Xaa can be any naturally occurring amino acid
  misc_feature (42)..(42): Xaa can be any naturally occurring amino acid
  misc_feature (51)..(51): Xaa can be any naturally occurring amino acid
  misc_feature (78)..(78): Xaa can be any naturally occurring amino acid
  misc_feature (82)..(83): Xaa can be any naturally occurring amino acid
  misc_feature (88)..(88): Xaa can be any naturally occurring amino acid
  misc_feature (100)..(101): Xaa can be any naturally occurring amino acid
  misc_feature (106)..(106): Xaa can be any naturally occurring amino acid
  misc_feature (109)..(110): Xaa can be any naturally occurring amino acid
  misc_feature (112)..(112): Xaa can be any naturally occurring amino acid
  misc_feature (114)..(114): Xaa can be any naturally occurring amino acid
  misc_feature (116)..(117): Xaa can be any naturally occurring amino acid
  misc_feature (120)..(120): Xaa can be any naturally occurring amino acid
  misc_feature (123)..(124): Xaa can be any naturally occurring amino acid
  misc_feature (131)..(131): Xaa can be any naturally occurring amino acid
  misc_feature (135)..(135): Xaa can be any naturally occurring amino acid
  misc_feature (139)..(140): Xaa can be any naturally occurring amino acid
  misc_feature (145)..(145): Xaa can be any naturally occurring amino acid
  misc_feature (164)..(164): Xaa can be any naturally occurring amino acid
VFFDLETAVXTXSGQPXAILEFGAILVCPMKLXELYSYXTLXRPTDLSLIXTLTKRRSGITRDGVLSAPTFSEIADEXYDIXXGRIWXGHNIKRFDCVRXXDAFAXIGXXPXEXKXXIDXLSXXSQKFGKXAGDXKMAXXATYFXLGDQAHRSLDDVRMNLEVXK

SEQ ID NO: 2 - protein, 165 aa, Boechera
  misc_feature (17)..(17): Xaa can be any naturally occurring amino acid
  misc_feature (83)..(83): Xaa can be any naturally occurring amino acid
  misc_feature (88)..(88): Xaa can be any naturally occurring amino acid
  misc_feature (101)..(101): Xaa can be any naturally occurring amino acid
  misc_feature (106)..(106): Xaa can be any naturally occurring amino acid
  misc_feature (110)..(110): Xaa can be any naturally occurring amino acid
  misc_feature (124)..(124): Xaa can be any naturally occurring amino acid
  misc_feature (140)..(140): Xaa can be any naturally occurring amino acid
  misc_feature (145)..(145): Xaa can be any naturally occurring amino acid
  misc_feature (164)..(164): Xaa can be any naturally occurring amino acid
VFFDLETAVPTKSGQPXAILEFGAILVCPMKLVELYSYSTLVRPTDLSLISTLTKRRSGITRDGVLSAPTFSEIADEVYDILXGRIWXGHNIKRFDCVRIXDAFAXIGLXPPEPKATIDSLSLXSQKFGKRAGDMKMASXATYFXLGDQAHRSLDDVRMNLEVXK

SEQ ID NO: 3 - protein, 165 aa, Boechera
  misc_feature (10)..(10): Xaa can be any naturally occurring amino acid
  misc_feature (12)..(12): Xaa can be any naturally occurring amino acid
  misc_feature (33)..(33): Xaa can be any naturally occurring amino acid
  misc_feature (39)..(39): Xaa can be any naturally occurring amino acid
  misc_feature (42)..(42): Xaa can be any naturally occurring amino acid
  misc_feature (51)..(51): Xaa can be any naturally occurring amino acid
  misc_feature (78)..(78): Xaa can be any naturally occurring amino acid
  misc_feature (82)..(82): Xaa can be any naturally occurring amino acid
  misc_feature (100)..(101): Xaa can be any naturally occurring amino acid
  misc_feature (106)..(106): Xaa can be any naturally occurring amino acid
  misc_feature (109)..(110): Xaa can be any naturally occurring amino acid
  misc_feature (112)..(112): Xaa can be any naturally occurring amino acid
  misc_feature (114)..(114): Xaa can be any naturally occurring amino acid
  misc_feature (116)..(117): Xaa can be any naturally occurring amino acid
  misc_feature (120)..(120): Xaa can be any naturally occurring amino acid
  misc_feature (123)..(124): Xaa can be any naturally occurring amino acid
  misc_feature (131)..(131): Xaa can be any naturally occurring amino acid
  misc_feature (135)..(135): Xaa can be any naturally occurring amino acid
  misc_feature (139)..(140): Xaa can be any naturally occurring amino acid
  misc_feature (145)..(145): Xaa can be any naturally occurring amino acid
  misc_feature (164)..(164): Xaa can be any naturally occurring amino acid
VFFDLETAVXTXSGQPFAILEFGAILVCPMKLXELYSYXTLXRPTDLSLIXTLTKRRSGITRDGVLSAPTFSEIADEXYDIXHGRIWAGHNIKRFDCVRXXDAFAXIGXXPXEXKXXIDXLSXXSQKFGKXAGDXKMAXXATYFXLGDQAHRSLDDVRMNLEVXK

SEQ ID NO: 4 - protein, 506 aa, Boechera
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
  misc_feature (23)..(23): Xaa can be any naturally occurring amino acid
  misc_feature (25)..(25): Xaa can be any naturally occurring amino acid
  misc_feature (30)..(30): Xaa can be any naturally occurring amino acid
  misc_feature (46)..(46): Xaa can be any naturally occurring amino acid
  misc_feature (52)..(52): Xaa can be any naturally occurring amino acid
  misc_feature (55)..(55): Xaa can be any naturally occurring amino acid
  misc_feature (64)..(64): Xaa can be any naturally occurring amino acid
  misc_feature (91)..(91): Xaa can be any naturally occurring amino acid
  misc_feature (95)..(96): Xaa can be any naturally occurring amino acid
  misc_feature (101)..(101): Xaa can be any naturally occurring amino acid
  misc_feature (113)..(114): Xaa can be any naturally occurring amino acid
  misc_feature (119)..(119): Xaa can be any naturally occurring amino acid
  misc_feature (122)..(123): Xaa can be any naturally occurring amino acid
  misc_feature (125)..(125): Xaa can be any naturally occurring amino acid
  misc_feature (127)..(127): Xaa can be any naturally occurring amino acid
  misc_feature (129)..(130): Xaa can be any naturally occurring amino acid
  misc_feature (133)..(133): Xaa can be any naturally occurring amino acid
  misc_feature (136)..(137): Xaa can be any naturally occurring amino acid
  misc_feature (144)..(144): Xaa can be any naturally occurring amino acid
  misc_feature (148)..(148): Xaa can be any naturally occurring amino acid
  misc_feature (152)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (181)..(181): Xaa can be any naturally occurring amino acid
  misc_feature (183)..(183): Xaa can be any naturally occurring amino acid
  misc_feature (195)..(197): Xaa can be any naturally occurring amino acid
  misc_feature (200)..(202): Xaa can be any naturally occurring amino acid
  misc_feature (206)..(207): Xaa can be any naturally occurring amino acid
  misc_feature (217)..(217): Xaa can be any naturally occurring amino acid
  misc_feature (228)..(228): Xaa can be any naturally occurring amino acid
  misc_feature (231)..(232): Xaa can be any naturally occurring amino acid
  misc_feature (240)..(240): Xaa can be any naturally occurring amino acid
  misc_feature (243)..(243): Xaa can be any naturally occurring amino acid
  misc_feature (258)..(259): Xaa can be any naturally occurring amino acid
  misc_feature (264)..(264): Xaa can be any naturally occurring amino acid
  misc_feature (285)..(285): Xaa can be any naturally occurring amino acid
  misc_feature (299)..(299): Xaa can be any naturally occurring amino acid
  misc_feature (303)..(303): Xaa can be any naturally occurring amino acid
  misc_feature (321)..(321): Xaa can be any naturally occurring amino acid
  misc_feature (328)..(328): Xaa can be any naturally occurring amino acid
  misc_feature (330)..(331): Xaa can be any naturally occurring amino acid
  misc_feature (336)..(336): Xaa can be any naturally occurring amino acid
  misc_feature (341)..(342): Xaa can be any naturally occurring amino acid
  misc_feature (348)..(348): Xaa can be any naturally occurring amino acid
  misc_feature (351)..(352): Xaa can be any naturally occurring amino acid
  misc_feature (354)..(354): Xaa can be any naturally occurring amino acid
  misc_feature (357)..(357): Xaa can be any naturally occurring amino acid
  misc_feature (360)..(361): Xaa can be any naturally occurring amino acid
  misc_feature (371)..(371): Xaa can be any naturally occurring amino acid
  misc_feature (387)..(387): Xaa can be any naturally occurring amino acid
  misc_feature (397)..(398): Xaa can be any naturally occurring amino acid
  misc_feature (400)..(400): Xaa can be any naturally occurring amino acid
  misc_feature (411)..(411): Xaa can be any naturally occurring amino acid
  misc_feature (413)..(414): Xaa can be any naturally occurring amino acid
  misc_feature (420)..(420): Xaa can be any naturally occurring amino acid
  misc_feature (422)..(422): Xaa can be any naturally occurring amino acid
  misc_feature (425)..(425): Xaa can be any naturally occurring amino acid
  misc_feature (431)..(431): Xaa can be any naturally occurring amino acid
  misc_feature (440)..(440): Xaa can be any naturally occurring amino acid
  misc_feature (442)..(442): Xaa can be any naturally occurring amino acid
  misc_feature (444)..(444): Xaa can be any naturally occurring amino acid
  misc_feature (448)..(448): Xaa can be any naturally occurring amino acid
  misc_feature (471)..(471): Xaa can be any naturally occurring amino acid
  misc_feature (474)..(474): Xaa can be any naturally occurring amino acid
  misc_feature (478)..(478): Xaa can be any naturally occurring amino acid
  misc_feature (482)..(482): Xaa can be any naturally occurring amino acid
  misc_feature (484)..(484): Xaa can be any naturally occurring amino acid
  misc_feature (488)..(488): Xaa can be any naturally occurring amino acid
  misc_feature (494)..(494): Xaa can be any naturally occurring amino acid
  misc_feature (503)..(503): Xaa can be any naturally occurring amino acid
MASTLGXDXRXEIVFFDLETAVXTXSGQPXAILEFGAILVCPMKLXELYSYXTLXRPTDLSLIXTLTKRRSGITRDGVLSAPTFSEIADEXYDIXXGRIWXGHNIKRFDCVRXXDAFAXIGXXPXEXKXXIDXLSXXSQKFGKXAGDXKMAXXATYFXLGDQAHRSLDDVRMNLEVXKXCXTXLFLESSVPDILXXXSWXXXRKSXXTRSNEKSLPXGVRESPTSSSXSPXXDPSSSSVXATXVKNHPIISLLTECSXXDTSSXEIDPSDITTLISKLHIGTLKXDAADEAKTVRDAAXEAKXVRQQGESTDPNAKDESFXGVNEVSXSXXRASLXPLYRXXLRMELXHNXXPXHLXWYXXKIRFGISRKXVDHVGRPKMNIVVDIXPDLCKILDAXXAXAHNLLIDSSTXSXXRPTVMXKXGFXNYPTAXLQISSESNXTXVXQKEXPLGTNQKLDFSSDNFEKLESALXPGXLVDXFFSXEXYDYXKMVGIXLAARKLVIXLKK

SEQ ID NO: 5 - protein, 506 aa, Boechera
  misc_feature (9)..(9): Xaa can be any naturally occurring amino acid
  misc_feature (30)..(30): Xaa can be any naturally occurring amino acid
  misc_feature (96)..(96): Xaa can be any naturally occurring amino acid
  misc_feature (101)..(101): Xaa can be any naturally occurring amino acid
  misc_feature (114)..(114): Xaa can be any naturally occurring amino acid
  misc_feature (119)..(119): Xaa can be any naturally occurring amino acid
  misc_feature (123)..(123): Xaa can be any naturally occurring amino acid
  misc_feature (137)..(137): Xaa can be any naturally occurring amino acid
  misc_feature (153)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (195)..(197): Xaa can be any naturally occurring amino acid
  misc_feature (200)..(201): Xaa can be any naturally occurring amino acid
  misc_feature (207)..(207): Xaa can be any naturally occurring amino acid
  misc_feature (217)..(217): Xaa can be any naturally occurring amino acid
  misc_feature (228)..(228): Xaa can be any naturally occurring amino acid
  misc_feature (231)..(231): Xaa can be any naturally occurring amino acid
  misc_feature (243)..(243): Xaa can be any naturally occurring amino acid
  misc_feature (258)..(258): Xaa can be any naturally occurring amino acid
  misc_feature (285)..(285): Xaa can be any naturally occurring amino acid
  misc_feature (303)..(303): Xaa can be any naturally occurring amino acid
  misc_feature (330)..(330): Xaa can be any naturally occurring amino acid
  misc_feature (341)..(342): Xaa can be any naturally occurring amino acid
  misc_feature (348)..(348): Xaa can be any naturally occurring amino acid
  misc_feature (352)..(352): Xaa can be any naturally occurring amino acid
  misc_feature (354)..(354): Xaa can be any naturally occurring amino acid
  misc_feature (387)..(387): Xaa can be any naturally occurring amino acid
  misc_feature (397)..(397): Xaa can be any naturally occurring amino acid
  misc_feature (411)..(411): Xaa can be any naturally occurring amino acid
  misc_feature (414)..(414): Xaa can be any naturally occurring amino acid
  misc_feature (420)..(420): Xaa can be any naturally occurring amino acid
  misc_feature (422)..(422): Xaa can be any naturally occurring amino acid
  misc_feature (425)..(425): Xaa can be any naturally occurring amino acid
  misc_feature (444)..(444): Xaa can be any naturally occurring amino acid
  misc_feature (478)..(478): Xaa can be any naturally occurring amino acid
  misc_feature (482)..(482): Xaa can be any naturally occurring amino acid
  misc_feature (484)..(484): Xaa can be any naturally occurring amino acid
  misc_feature (494)..(494): Xaa can be any naturally occurring amino acid
MASTLGGDXRNEIVFFDLETAVPTKSGQPXAILEFGAILVCPMKLVELYSYSTLVRPTDLSLISTLTKRRSGITRDGVLSAPTFSEIADEVYDILXGRIWXGHNIKRFDCVRIXDAFAXIGLXPPEPKATIDSLSLXSQKFGKRAGDMKMASXATYFXLGDQAHRSLDDVRMNLEVXKXCSTVLFLESSVPDILXXXSWXXPRKSPXTRSNEKSLPXGVRESPTSSSXSPXTDPSSSSVDATXVKNHPIISLLTECSXSDTSSCEIDPSDITTLISKLHIGTLKXDAADEAKTVRDAADEAKXVRQQGESTDPNAKDESFLGVNEVSVSXIRASLIPLYRXXLRMELXHNDXPXHLCWYSLKIRFGISRKYVDHVGRPKMNIVVDIXPDLCKILDAXDAAAHNLLIDSSTXSDXRPTVMXKXGFXNYPTARLQISSESNGTQVXQKEEPLGTNQKLDFSSDNFEKLESALLPGTLVDXFFSXEXYDYKKMVGIXLAARKLVIQLKK

SEQ ID NO: 6 - protein, 506 aa, Boechera
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
  misc_feature (23)..(23): Xaa can be any naturally occurring amino acid
  misc_feature (25)..(25): Xaa can be any naturally occurring amino acid
  misc_feature (46)..(46): Xaa can be any naturally occurring amino acid
  misc_feature (52)..(52): Xaa can be any naturally occurring amino acid
  misc_feature (55)..(55): Xaa can be any naturally occurring amino acid
  misc_feature (64)..(64): Xaa can be any naturally occurring amino acid
  misc_feature (91)..(91): Xaa can be any naturally occurring amino acid
  misc_feature (95)..(95): Xaa can be any naturally occurring amino acid
  misc_feature (113)..(114): Xaa can be any naturally occurring amino acid
  misc_feature (119)..(119): Xaa can be any naturally occurring amino acid
  misc_feature (122)..(123): Xaa can be any naturally occurring amino acid
  misc_feature (125)..(125): Xaa can be any naturally occurring amino acid
  misc_feature (127)..(127): Xaa can be any naturally occurring amino acid
  misc_feature (129)..(130): Xaa can be any naturally occurring amino acid
  misc_feature (133)..(133): Xaa can be any naturally occurring amino acid
  misc_feature (136)..(137): Xaa can be any naturally occurring amino acid
  misc_feature (144)..(144): Xaa can be any naturally occurring amino acid
  misc_feature (148)..(148): Xaa can be any naturally occurring amino acid
  misc_feature (152)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (181)..(181): Xaa can be any naturally occurring amino acid
  misc_feature (183)..(183): Xaa can be any naturally occurring amino acid
  misc_feature (195)..(196): Xaa can be any naturally occurring amino acid
  misc_feature (200)..(202): Xaa can be any naturally occurring amino acid
  misc_feature (206)..(206): Xaa can be any naturally occurring amino acid
  misc_feature (232)..(232): Xaa can be any naturally occurring amino acid
  misc_feature (240)..(240): Xaa can be any naturally occurring amino acid
  misc_feature (243)..(243): Xaa can be any naturally occurring amino acid
  misc_feature (259)..(259): Xaa can be any naturally occurring amino acid
  misc_feature (264)..(264): Xaa can be any naturally occurring amino acid
  misc_feature (285)..(285): Xaa can be any naturally occurring amino acid
  misc_feature (299)..(299): Xaa can be any naturally occurring amino acid
  misc_feature (303)..(303): Xaa can be any naturally occurring amino acid
  misc_feature (321)..(321): Xaa can be any naturally occurring amino acid
  misc_feature (328)..(328): Xaa can be any naturally occurring amino acid
  misc_feature (331)..(331): Xaa can be any naturally occurring amino acid
  misc_feature (336)..(336): Xaa can be any naturally occurring amino acid
  misc_feature (341)..(342): Xaa can be any naturally occurring amino acid
  misc_feature (348)..(348): Xaa can be any naturally occurring amino acid
  misc_feature (351)..(351): Xaa can be any naturally occurring amino acid
  misc_feature (357)..(357): Xaa can be any naturally occurring amino acid
  misc_feature (360)..(361): Xaa can be any naturally occurring amino acid
  misc_feature (371)..(371): Xaa can be any naturally occurring amino acid
  misc_feature (397)..(398): Xaa can be any naturally occurring amino acid
  misc_feature (400)..(400): Xaa can be any naturally occurring amino acid
  misc_feature (411)..(411): Xaa can be any naturally occurring amino acid
  misc_feature (413)..(414): Xaa can be any naturally occurring amino acid
  misc_feature (420)..(420): Xaa can be any naturally occurring amino acid
  misc_feature (422)..(422): Xaa can be any naturally occurring amino acid
  misc_feature (431)..(431): Xaa can be any naturally occurring amino acid
  misc_feature (440)..(440): Xaa can be any naturally occurring amino acid
  misc_feature (442)..(442): Xaa can be any naturally occurring amino acid
  misc_feature (444)..(444): Xaa can be any naturally occurring amino acid
  misc_feature (448)..(448): Xaa can be any naturally occurring amino acid
  misc_feature (471)..(471): Xaa can be any naturally occurring amino acid
  misc_feature (474)..(474): Xaa can be any naturally occurring amino acid
  misc_feature (478)..(478): Xaa can be any naturally occurring amino acid
  misc_feature (482)..(482): Xaa can be any naturally occurring amino acid
  misc_feature (484)..(484): Xaa can be any naturally occurring amino acid
  misc_feature (488)..(488): Xaa can be any naturally occurring amino acid
  misc_feature (503)..(503): Xaa can be any naturally occurring amino acid
MASTLGXDXRXEIVFFDLETAVXTXSGQPFAILEFGAILVCPMKLXELYSYXTLXRPTDLSLIXTLTKRRSGITRDGVLSAPTFSEIADEXYDIXHGRIWAGHNIKRFDCVRXXDAFAXIGXXPXEXKXXIDXLSXXSQKFGKXAGDXKMAXXATYFXLGDQAHRSLDDVRMNLEVXKXCXTXLFLESSVPDILXXMSWXXXRKSXRTRSNEKSLPNGVRESPTSSSSSPKXDPSSSSVXATXVKNHPIISLLTECSEXDTSSXEIDPSDITTLISKLHIGTLKXDAADEAKTVRDAAXEAKXVRQQGESTDPNAKDESFXGVNEVSXSSXRASLXPLYRXXLRMELXHNXTPLHLXWYXXKIRFGISRKXVDHVGRPKMNIVVDIPPDLCKILDAXXAXAHNLLIDSSTXSXXRPTVMXKXGFANYPTAXLQISSESNXTXVXQKEXPLGTNQKLDFSSDNFEKLESALXPGXLVDXFFSXEXYDYXKMVGIRLAARKLVIXLKK

SEQ ID NO: 7 - protein, 496 aa, Boechera
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
  misc_feature (23)..(23): Xaa can be any naturally occurring amino acid
  misc_feature (25)..(25): Xaa can be any naturally occurring amino acid
  misc_feature (30)..(30): Xaa can be any naturally occurring amino acid
  misc_feature (46)..(46): Xaa can be any naturally occurring amino acid
  misc_feature (52)..(52): Xaa can be any naturally occurring amino acid
  misc_feature (55)..(55): Xaa can be any naturally occurring amino acid
  misc_feature (64)..(64): Xaa can be any naturally occurring amino acid
  misc_feature (91)..(91): Xaa can be any naturally occurring amino acid
  misc_feature (95)..(96): Xaa can be any naturally occurring amino acid
  misc_feature (101)..(101): Xaa can be any naturally occurring amino acid
  misc_feature (113)..(114): Xaa can be any naturally occurring amino acid
  misc_feature (119)..(119): Xaa can be any naturally occurring amino acid
  misc_feature (122)..(123): Xaa can be any naturally occurring amino acid
  misc_feature (125)..(125): Xaa can be any naturally occurring amino acid
  misc_feature (127)..(127): Xaa can be any naturally occurring amino acid
  misc_feature (129)..(130): Xaa can be any naturally occurring amino acid
  misc_feature (133)..(133): Xaa can be any naturally occurring amino acid
  misc_feature (136)..(137): Xaa can be any naturally occurring amino acid
  misc_feature (144)..(144): Xaa can be any naturally occurring amino acid
  misc_feature (148)..(148): Xaa can be any naturally occurring amino acid
  misc_feature (152)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (181)..(181): Xaa can be any naturally occurring amino acid
  misc_feature (183)..(183): Xaa can be any naturally occurring amino acid
  misc_feature (195)..(197): Xaa can be any naturally occurring amino acid
  misc_feature (200)..(202): Xaa can be any naturally occurring amino acid
  misc_feature (206)..(207): Xaa can be any naturally occurring amino acid
  misc_feature (217)..(217): Xaa can be any naturally occurring amino acid
  misc_feature (228)..(228): Xaa can be any naturally occurring amino acid
  misc_feature (231)..(232): Xaa can be any naturally occurring amino acid
  misc_feature (240)..(240): Xaa can be any naturally occurring amino acid
  misc_feature (243)..(243): Xaa can be any naturally occurring amino acid
  misc_feature (258)..(259): Xaa can be any naturally occurring amino acid
  misc_feature (264)..(264): Xaa can be any naturally occurring amino acid
  misc_feature (285)..(285): Xaa can be any naturally occurring amino acid
  misc_feature (289)..(289): Xaa can be any naturally occurring amino acid
  misc_feature (293)..(293): Xaa can be any naturally occurring amino acid
  misc_feature (311)..(311): Xaa can be any naturally occurring amino acid
  misc_feature (318)..(318): Xaa can be any naturally occurring amino acid
  misc_feature (320)..(321): Xaa can be any naturally occurring amino acid
  misc_feature (326)..(326): Xaa can be any naturally occurring amino acid
  misc_feature (331)..(332): Xaa can be any naturally occurring amino acid
  misc_feature (338)..(338): Xaa can be any naturally occurring amino acid
  misc_feature (341)..(342): Xaa can be any naturally occurring amino acid
  misc_feature (344)..(344): Xaa can be any naturally occurring amino acid
  misc_feature (347)..(347): Xaa can be any naturally occurring amino acid
  misc_feature (350)..(351): Xaa can be any naturally occurring amino acid
  misc_feature (361)..(361): Xaa can be any naturally occurring amino acid
  misc_feature (377)..(377): Xaa can be any naturally occurring amino acid
  misc_feature (387)..(388): Xaa can be any naturally occurring amino acid
  misc_feature (390)..(390): Xaa can be any naturally occurring amino acid
  misc_feature (401)..(401): Xaa can be any naturally occurring amino acid
  misc_feature (403)..(404): Xaa can be any naturally occurring amino acid
  misc_feature (410)..(410): Xaa can be any naturally occurring amino acid
  misc_feature (412)..(412): Xaa can be any naturally occurring amino acid
  misc_feature (415)..(415): Xaa can be any naturally occurring amino acid
  misc_feature (421)..(421): Xaa can be any naturally occurring amino acid
  misc_feature (430)..(430): Xaa can be any naturally occurring amino acid
  misc_feature (432)..(432): Xaa can be any naturally occurring amino acid
  misc_feature (434)..(434): Xaa can be any naturally occurring amino acid
  misc_feature (438)..(438): Xaa can be any naturally occurring amino acid
  misc_feature (461)..(461): Xaa can be any naturally occurring amino acid
  misc_feature (464)..(464): Xaa can be any naturally occurring amino acid
  misc_feature (468)..(468): Xaa can be any naturally occurring amino acid
  misc_feature (472)..(472): Xaa can be any naturally occurring amino acid
  misc_feature (474)..(474): Xaa can be any naturally occurring amino acid
  misc_feature (478)..(478): Xaa can be any naturally occurring amino acid
  misc_feature (484)..(484): Xaa can be any naturally occurring amino acid
  misc_feature (493)..(493): Xaa can be any naturally occurring amino acid
MASTLGXDXRXEIVFFDLETAVXTXSGQPXAILEFGAILVCPMKLXELYSYXTLXRPTDLSLIXTLTKRRSGITRDGVLSAPTFSEIADEXYDIXXGRIWXGHNIKRFDCVRXXDAFAXIGXXPXEXKXXIDXLSXXSQKFGKXAGDXKMAXXATYFXLGDQAHRSLDDVRMNLEVXKXCXTXLFLESSVPDILXXXSWXXXRKSXXTRSNEKSLPXGVRESPTSSSXSPXXDPSSSSVXATXVKNHPIISLLTECSXXDTSSXEIDPSDITTLISKLHIGTLKXDAAXEAKXVRQQGESTDPNAKDESFXGVNEVSXSXXRASLXPLYRXXLRMELXHNXXPXHLXWYXXKIRFGISRKXVDHVGRPKMNIVVDIXPDLCKILDAXXAXAHNLLIDSSTXSXXRPTVMXKXGFXNYPTAXLQISSESNXTXVXQKEXPLGTNQKLDFSSDNFEKLESALXPGXLVDXFFSXEXYDYXKMVGIXLAARKLVIXLKK

SEQ ID NO: 8 - protein, 496 aa, Boechera
  misc_feature (9)..(9): Xaa can be any naturally occurring amino acid
  misc_feature (30)..(30): Xaa can be any naturally occurring amino acid
  misc_feature (96)..(96): Xaa can be any naturally occurring amino acid
  misc_feature (101)..(101): Xaa can be any naturally occurring amino acid
  misc_feature (114)..(114): Xaa can be any naturally occurring amino acid
  misc_feature (119)..(119): Xaa can be any naturally occurring amino acid
  misc_feature (123)..(123): Xaa can be any naturally occurring amino acid
  misc_feature (137)..(137): Xaa can be any naturally occurring amino acid
  misc_feature (153)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (195)..(197): Xaa can be any naturally occurring amino acid
  misc_feature (200)..(201): Xaa can be any naturally occurring amino acid
  misc_feature (207)..(207): Xaa can be any naturally occurring amino acid
  misc_feature (217)..(217): Xaa can be any naturally occurring amino acid
  misc_feature (228)..(228): Xaa can be any naturally occurring amino acid
  misc_feature (231)..(231): Xaa can be any naturally occurring amino acid
  misc_feature (243)..(243): Xaa can be any naturally occurring amino acid
  misc_feature (258)..(258): Xaa can be any naturally occurring amino acid
  misc_feature (285)..(285): Xaa can be any naturally occurring amino acid
  misc_feature (293)..(293): Xaa can be any naturally occurring amino acid
  misc_feature (320)..(320): Xaa can be any naturally occurring amino acid
  misc_feature (331)..(332): Xaa can be any naturally occurring amino acid
  misc_feature (338)..(338): Xaa can be any naturally occurring amino acid
  misc_feature (342)..(342): Xaa can be any naturally occurring amino acid
  misc_feature (344)..(344): Xaa can be any naturally occurring amino acid
  misc_feature (377)..(377): Xaa can be any naturally occurring amino acid
  misc_feature (387)..(387): Xaa can be any naturally occurring amino acid
  misc_feature (401)..(401): Xaa can be any naturally occurring amino acid
  misc_feature (404)..(404): Xaa can be any naturally occurring amino acid
  misc_feature (410)..(410): Xaa can be any naturally occurring amino acid
  misc_feature (412)..(412): Xaa can be any naturally occurring amino acid
  misc_feature (415)..(415): Xaa can be any naturally occurring amino acid
  misc_feature (434)..(434): Xaa can be any naturally occurring amino acid
  misc_feature (468)..(468): Xaa can be any naturally occurring amino acid
  misc_feature (472)..(472): Xaa can be any naturally occurring amino acid
  misc_feature (474)..(474): Xaa can be any naturally occurring amino acid
  misc_feature (484)..(484): Xaa can be any naturally occurring amino acid
MASTLGGDXRNEIVFFDLETAVPTKSGQPXAILEFGAILVCPMKLVELYSYSTLVRPTDLSLISTLTKRRSGITRDGVLSAPTFSEIADEVYDILXGRIWXGHNIKRFDCVRIXDAFAXIGLXPPEPKATIDSLSLXSQKFGKRAGDMKMASXATYFXLGDQAHRSLDDVRMNLEVXKXCSTVLFLESSVPDILXXXSWXXPRKSPXTRSNEKSLPXGVRESPTSSSXSPXTDPSSSSVDATXVKNHPIISLLTECSXSDTSSCEIDPSDITTLISKLHIGTLKXDAADEAKXVRQQGESTDPNAKDESFLGVNEVSVSXIRASLIPLYRXXLRMELXHNDXPXHLCWYSLKIRFGISRKYVDHVGRPKMNIVVDIXPDLCKILDAXDAAAHNLLIDSSTXSDXRPTVMXKXGFXNYPTARLQISSESNGTQVXQKEEPLGTNQKLDFSSDNFEKLESALLPGTLVDXFFSXEXYDYKKMVGIXLAARKLVIQLKK

SEQ ID NO: 9 - protein, 496 aa, Boechera
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
  misc_feature (23)..(23): Xaa can be any naturally occurring amino acid
  misc_feature (25)..(25): Xaa can be any naturally occurring amino acid
  misc_feature (46)..(46): Xaa can be any naturally occurring amino acid
  misc_feature (52)..(52): Xaa can be any naturally occurring amino acid
  misc_feature (55)..(55): Xaa can be any naturally occurring amino acid
  misc_feature (64)..(64): Xaa can be any naturally occurring amino acid
  misc_feature (91)..(91): Xaa can be any naturally occurring amino acid
  misc_feature (95)..(95): Xaa can be any naturally occurring amino acid
  misc_feature (113)..(114): Xaa can be any naturally occurring amino acid
  misc_feature (119)..(119): Xaa can be any naturally occurring amino acid
  misc_feature (122)..(123): Xaa can be any naturally occurring amino acid
  misc_feature (125)..(125): Xaa can be any naturally occurring amino acid
  misc_feature (127)..(127): Xaa can be any naturally occurring amino acid
  misc_feature (129)..(130): Xaa can be any naturally occurring amino acid
  misc_feature (133)..(133): Xaa can be any naturally occurring amino acid
  misc_feature (136)..(137): Xaa can be any naturally occurring amino acid
  misc_feature (144)..(144): Xaa can be any naturally occurring amino acid
  misc_feature (148)..(148): Xaa can be any naturally occurring amino acid
  misc_feature (152)..(153): Xaa can be any naturally occurring amino acid
  misc_feature (158)..(158): Xaa can be any naturally occurring amino acid
  misc_feature (177)..(177): Xaa can be any naturally occurring amino acid
  misc_feature (179)..(179): Xaa can be any naturally occurring amino acid
  misc_feature (181)..(181): Xaa can be any naturally occurring amino acid
  misc_feature (183)..(183): Xaa can be any naturally occurring amino acid
  misc_feature (195)..(196): Xaa can be any naturally occurring amino acid
  misc_feature (200)..(202): Xaa can be any naturally occurring amino acid
  misc_feature (206)..(206): Xaa can be any naturally occurring amino acid
  misc_feature (232)..(232): Xaa can be any naturally occurring amino acid
  misc_feature (240)..(240): Xaa can be any naturally occurring amino acid
  misc_feature (243)..(243): Xaa can be any naturally occurring amino acid
  misc_feature (259)..(259): Xaa can be any naturally occurring amino acid
  misc_feature (264)..(264): Xaa can be any naturally occurring amino acid
  misc_feature (285)..(285): Xaa can be any naturally occurring amino acid
  misc_feature (289)..(289): Xaa can be any naturally occurring amino acid
  misc_feature (293)..(293): Xaa can be any naturally occurring amino acid
  misc_feature (311)..(311): Xaa can be any naturally occurring amino acid
  misc_feature (318)..(318): Xaa can be any naturally occurring amino acid
  misc_feature (321)..(321): Xaa can be any naturally occurring amino acid
  misc_feature (326)..(326): Xaa can be any naturally occurring amino acid
  misc_feature (331)..(332): Xaa can be any naturally occurring amino acid
  misc_feature (338)..(338): Xaa can be any naturally occurring amino acid
  misc_feature (341)..(341): Xaa can be any naturally occurring amino acid
  misc_feature (347)..(347): Xaa can be any naturally occurring amino acid
  misc_feature (350)..(351): Xaa can be any naturally occurring amino acid
  misc_feature (361)..(361): Xaa can be any naturally occurring amino acid
  misc_feature (387)..(388): Xaa can be any naturally occurring amino acid
  misc_feature (390)..(390): Xaa can be any naturally occurring amino acid
  misc_feature (401)..(401): Xaa can be any naturally occurring amino acid
  misc_feature (403)..(404): Xaa can be any naturally occurring amino acid
  misc_feature (410)..(410): Xaa can be any naturally occurring amino acid
  misc_feature (412)..(412): Xaa can be any naturally occurring amino acid
  misc_feature (421)..(421): Xaa can be any naturally occurring amino acid
  misc_feature (430)..(430): Xaa can be any naturally occurring amino acid
  misc_feature (432)..(432): Xaa can be any naturally occurring amino acid
  misc_feature (434)..(434): Xaa can be any naturally occurring amino acid
  misc_feature (438)..(438): Xaa can be any naturally occurring amino acid
  misc_feature (461)..(461): Xaa can be any naturally occurring amino acid
  misc_feature (464)..(464): Xaa can be any naturally occurring amino acid
  misc_feature (468)..(468): Xaa can be any naturally occurring amino acid
  misc_feature (472)..(472): Xaa can be any naturally occurring amino acid
  misc_feature (474)..(474): Xaa can be any naturally occurring amino acid
  misc_feature (478)..(478): Xaa can be any naturally occurring amino acid
  misc_feature (493)..(493): Xaa can be any naturally occurring amino acid
MASTLGXDXRXEIVFFDLETAVXTXSGQPFAILEFGAILVCPMKLXELYSYXTLXRPTDLSLIXTLTKRRSGITRDGVLSAPTFSEIADEXYDIXHGRIWAGHNIKRFDCVRXXDAFAXIGXXPXEXKXXIDXLSXXSQKFGKXAGDXKMAXXATYFXLGDQAHRSLDDVRMNLEVXKXCXTXLFLESSVPDILXXMSWXXXRKSXRTRSNEKSLPNGVRESPTSSSSSPKXDPSSSSVXATXVKNHPIISLLTECSEXDTSSXEIDPSDITTLISKLHIGTLKXDAAXEAKXVRQQGESTDPNAKDESFXGVNEVSXSSXRASLXPLYRXXLRMELXHNXTPLHLXWYXXKIRFGISRKXVDHVGRPKMNIVVDIPPDLCKILDAXXAXAHNLLIDSSTXSXXRPTVMXKXGFANYPTAXLQISSESNXTXVXQKEXPLGTNQKLDFSSDNFEKLESALXPGXLVDXFFSXEXYDYXKMVGIRLAARKLVIXLKK

SEQ ID NO: 10 - protein, 165 aa, Boechera
VFFDLETAVPTKSGQPFAILEFGAILVCPMKLVELYSYSTLVRPTDLSLISTLTKRRSGITRDGVLSAPTFSEIADEVYDILHGRIWVGHNIKRFDCVRIRDAFAEIGLPPPEPKATIDSLSLLSQKFGKRAGDMKMASHATYFGLGDQAHRSLDDVRMNLEVIK

SEQ ID NO: 11 - protein, 165 aa, Boechera
VFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAEIGLPPPEPKATIDSLSLLSQKFGKRAGDMKMASLATYFGLGDQAHRSLDDVRMNLEVIK

SEQ ID NO: 12 - protein, 165 aa, Boechera; identical to SEQ ID NO: 11

SEQ ID NO: 13 - protein, 496 aa, Boechera
MASTLGGDERNEIVFFDLETAVPTKSGQPFAILEFGAILVCPMKLVELYSYSTLVRPTDLSLISTLTKRRSGITRDGVLSAPTFSEIADEVYDILHGRIWVGHNIKRFDCVRIRDAFAEIGLPPPEPKATIDSLSLLSQKFGKRAGDMKMASHATYFGLGDQAHRSLDDVRMNLEVIKHCSTVLFLESSVPDILTDMSWLFPRKSPRTRSNEKSLPNGVRESPTSSSSSPKTDPSSSSVDATAVKNHPIISLLTECSESDTSSCEIDPSDITTLISKLHIGTLKTDAADEAKTVRQQGESTDPNAKDESFLGVNEVSVSSIRASLIPLYRRSLRMELFHNDTPLHLCWYSLKIRFGISRKYVDHVGRPKMNIVVDIPPDLCKILDASDAAAHNLLIDSSTSSDWRPTVMRKKGFANYPTARLQISSESNGTQVHQKEEPLGTNQKLDFSSDNFEKLESALLPGTLVDAFFSLEPYDYKKMVGIRLAARKLVIHLKK

SEQ ID NO: 14 - protein, 496 aa, Boechera
MASTLGGDERNEIVFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAEIGLPPPEPKATIDSLSLLSQKFGKRAGDMKMASLATYFGLGDQAHRSLDDVRMNLEVIKHCSTVLFLESSVPDILTDMSWLFPRKSPRTRSNEKSLPNGVRESPTSSSSSPQTDPSSSSVDATAVKNHPIISLLTECSESDTSSCEIDPSDITTLISKLHIGTLKTDAADEAKTVRQQGESTDPNAKDESFLGVNEVSVSSIRASLIPLYRRSLRMELFHNDTPLHLCWYSLKIRFGISRKYVDHVGRPKMNIVVDIPPDLCKILDASDAAAHNLLIDSSTSSDWRPTVMRKKGFANYPTARLQISSESNGTQVYQKEEPLGTNQKLDFSSDNFEKLESALLPGTLVDVFFSVEPYDYKKMVGIRLAARKLVIQLKK

SEQ ID NO: 15 - protein, 496 aa, Boechera
MASTLGGDERNEIVFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAEIGLPPPEPKATIDSLSLLSQKFGKRAGDMKMASLATYFGLGDQAHRSLDDVRMNLEVIKHCATVLFLESSVPDILTDMSWLFPRKSPRTRSNEKSLPNGVRESPTSSSSSPKTDPSSSSVDATAVKNHPIISLLTECSESDTSSCEIDPSDITTLISKLHIGTLKRDAADEAKIVRQQGESTDPNAKDESFLGVNEVSVSSIRASLIPLYRGSLRMELFHNDTPLHLCWYSLKIRFGISRKYVDHVGRPKMNIVVDIPPDLCKILDAYDAAAHNLLIDSSTSSDWRPTVMRKEGFANYPTARLQISSESNGTQVYQKEEPLGTNQKLDFSSDNFEKLESALLPGTLVDAFFSPESYDYKKMVGIRLAARKLVIHLKK

SEQ ID NO: 16 - protein, 165 aa, Boechera
VFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAGIGVSPPEPKATIDSLSLLSQKFGKRAGDMKMASLATYFGLGDQAHRSLDDVRMNLEVVK

SEQ ID NO: 17 - protein, 165 aa, Boechera
VFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAGIGLSPPEPKATIDSLSLLSQKFGKRAGDMKMASLATYFGLGDQAHRSLDDVRMNLEVVK

SEQ ID NO: 18 - protein, 165 aa, Boechera; identical to SEQ ID NO: 17

SEQ ID NO: 19 - protein, 506 aa, Boechera
MASTLGGDERCEIVFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAGIGVSPPEPKATIDSLSLLSQKFGKRAGDMKMASLATYFGLGDQAHRSLDDVRMNLEVVKYCATVLFLESSVPDILKDMSWFSPRKSPRTRSNEKSLPNGVRESPTSSSSSPKTDPSSSSVDATTVKNHPIISLLTECSESDTSSCEIDPSDITTLISKLHIGTLKRDAADEAKTVRDAADEAKTVRQQGESTDPNAKDESFLGVNEVSVSSIRASLIPLYRGSLRMELFHNDTPLHLCWYSLKIRFGISRKYVDHVGRPKMNIVVDIPPDLCKILDASDAAAHNLLIDSSTSSDWRPTVMRKEGFANYPTARLQISSESNGTQVHQKEEPLGTNQKLDFSSDNFEKLESALLPGTLVDAFFSLEPYDYKKMVGIRLAARKLVIHLKK

SEQ ID NO: 20 - protein, 496 aa, Boechera
MASTLGGDERCEIVFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAGIGLSPPEPKATIDSLSLLSQKFGKRAGDMKMASLATYFGLGDQAHRSLDDVRMNLEVVKYCATVLFLESSVPDILKDMSWFSPRKSPRTRSNEKSLPNGVRESPTSSSSSPKTDPSSSSVDATTVKNHPIISLLTECSESDTSSCEIDPSDITTLISKLHIGTLKRDAADEAKIVRQQGESTDPNAKDESFLGVNEVSVSSIRASLIPLYRGSLRMELLHNDTPLHLCWYSLKIRFGISRKYVDHVGRPKMNIVVDIPPDLCKILDAYDAAAHNLLIDSSTSSDWRPTVMRKEGFANYPTARLQISSESNGTQVYQKEEPLGTNQKLDFSSDNFEKLESALLPGTLVDAFFSLESYDYKKMVGIRLAARKLVIHLKK

SEQ ID NO: 21 - protein, 496 aa, Boechera
MASTLGGDGRCEIVFFDLETAVPTKSGQPFAILEFGAILVCPMKLVELYSYSTLVRPTDLSLISTLTKRRSGITRDGVLSAPTFSEIADEVYDILHGRIWAGHNIKRFDCVRIRDAFAGIGLSPPEPKATIDSLSLLSQKFGKRAGDMKMASLATYFGLGDQAHRSLDDVRMNLEVVKYCATVLFLESSVPDILKDMSWFSPRKSPRTRSNEKSLPNGVRESPTSSSSSPKTDPSSSSVDATTVKNHPIISLLTECSESDTSSCEIDPSDITTLISKLHIGTLKRDAADEAKTVRQQGESTDPNAKDESFLGVNEVSVSSIRASLIPLYRGGLRMELFHNDTPLHLRWYSLKIRFGISRKYVDHVGRPKMNIVVDIPPDLCKILDASDAAAHNLLIDSSTSSDWRPTVMRKEGFANYPTARLQISSESNGTQVHQKEEPLGTNQKLDFSSDNFEKLESALLPGTLVDAFFSLEPYDYKKMVGIRLAARKLVIHLKK

SEQ ID NO: 22 - DNA, 2423 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (89)..(89): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (177)..(177): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (287)..(287): n is a, c, g, or t
  misc_feature (303)..(304): n is a, c, g, or t
  misc_feature (312)..(317): n is a, c, g, or t
  misc_feature (320)..(321): n is a, c, g, or t
  misc_feature (323)..(323): n is a, c, g, or t
  misc_feature (325)..(326): n is a, c, g, or t
  misc_feature (334)..(334): n is a, c, g, or t
  misc_feature (337)..(337): n is a, c, g, or t
  misc_feature (343)..(343): n is a, c, g, or t
  misc_feature (346)..(346): n is a, c, g, or t
  misc_feature (385)..(386): n is a, c, g, or t
  misc_feature (420)..(420): n is a, c, g, or t
  misc_feature (423)..(423): n is a, c, g, or t
  misc_feature (439)..(439): n is a, c, g, or t
  misc_feature (447)..(447): n is a, c, g, or t
  misc_feature (450)..(450): n is a, c, g, or t
  misc_feature (457)..(458): n is a, c, g, or t
  misc_feature (462)..(462): n is a, c, g, or t
  misc_feature (464)..(464): n is a, c, g, or t
  misc_feature (469)..(469): n is a, c, g, or t
  misc_feature (471)..(471): n is a, c, g, or t
  misc_feature (480)..(480): n is a, c, g, or t
  misc_feature (491)..(493): n is a, c, g, or t
  misc_feature (513)..(513): n is a, c, g, or t
  misc_feature (521)..(521): n is a, c, g, or t
  misc_feature (525)..(525): n is a, c, g, or t
  misc_feature (551)..(551): n is a, c, g, or t
  misc_feature (572)..(572): n is a, c, g, or t
  misc_feature (580)..(580): n is a, c, g, or t
  misc_feature (590)..(590): n is a, c, g, or t
  misc_feature (608)..(608): n is a, c, g, or t
  misc_feature (610)..(610): n is a, c, g, or t
  misc_feature (620)..(620): n is a, c, g, or t
  misc_feature (638)..(638): n is a, c, g, or t
  misc_feature (642)..(642): n is a, c, g, or t
  misc_feature (655)..(655): n is a, c, g, or t
  misc_feature (658)..(662): n is a, c, g, or t
  misc_feature (665)..(665): n is a, c, g, or t
  misc_feature (668)..(668): n is a, c, g, or t
  misc_feature (678)..(678): n is a, c, g, or t
  misc_feature (680)..(680): n is a, c, g, or t
  misc_feature (683)..(683): n is a, c, g, or t
  misc_feature (690)..(690): n is a, c, g, or t
  misc_feature (698)..(698): n is a, c, g, or t
  misc_feature (708)..(708): n is a, c, g, or t
  misc_feature (711)..(711): n is a, c, g, or t
  misc_feature (722)..(722): n is a, c, g, or t
  misc_feature (726)..(727): n is a, c, g, or t
  misc_feature (740)..(741): n is a, c, g, or t
  misc_feature (760)..(760): n is a, c, g, or t
  misc_feature (769)..(769): n is a, c, g, or t
  misc_feature (791)..(791): n is a, c, g, or t
  misc_feature (794)..(794): n is a, c, g, or t
  misc_feature (801)..(801): n is a, c, g, or t
  misc_feature (838)..(839): n is a, c, g, or t
  misc_feature (845)..(845): n is a, c, g, or t
  misc_feature (851)..(851): n is a, c, g, or t
  misc_feature (857)..(857): n is a, c, g, or t
  misc_feature (862)..(862): n is a, c, g, or t
  misc_feature (875)..(875): n is a, c, g, or t
  misc_feature (882)..(882): n is a, c, g, or t
  misc_feature (902)..(902): n is a, c, g, or t
  misc_feature (908)..(908): n is a, c, g, or t
  misc_feature (912)..(912): n is a, c, g, or t
  misc_feature (915)..(915): n is a, c, g, or t
  misc_feature (923)..(923): n is a, c, g, or t
  misc_feature (931)..(931): n is a, c, g, or t
  misc_feature (934)..(934): n is a, c, g, or t
  misc_feature (939)..(940): n is a, c, g, or t
  misc_feature (952)..(952): n is a, c, g, or t
  misc_feature (961)..(961): n is a, c, g, or t
  misc_feature (963)..(963): n is a, c, g, or t
  misc_feature (967)..(967): n is a, c, g, or t
  misc_feature (970)..(970): n is a, c, g, or t
  misc_feature (976)..(977): n is a, c, g, or t
  misc_feature (980)..(980): n is a, c, g, or t
  misc_feature (983)..(983): n is a, c, g, or t
  misc_feature (993)..(993): n is a, c, g, or t
  misc_feature (1003)..(1003): n is a, c, g, or t
  misc_feature (1025)..(1025): n is a, c, g, or t
  misc_feature (1049)..(1050): n is a, c, g, or t
  misc_feature (1089)..(1089): n is a, c, g, or t
  misc_feature (1095)..(1095): n is a, c, g, or t
  misc_feature (1098)..(1098): n is a, c, g, or t
  misc_feature (1109)..(1109): n is a, c, g, or t
  misc_feature (1111)..(1112): n is a, c, g, or t
  misc_feature (1116)..(1116): n is a, c, g, or t
  misc_feature (1125)..(1125): n is a, c, g, or t
  misc_feature (1127)..(1127): n is a, c, g, or t
  misc_feature (1130)..(1130): n is a, c, g, or t
  misc_feature (1142)..(1142): n is a, c, g, or t
  misc_feature (1144)..(1144): n is a, c, g, or t
  misc_feature (1173)..(1174): n is a, c, g, or t
  misc_feature (1194)..(1194): n is a, c, g, or t
  misc_feature (1209)..(1209): n is a, c, g, or t
  misc_feature (1216)..(1216): n is a, c, g, or t
  misc_feature (1219)..(1219): n is a, c, g, or t
  misc_feature (1224)..(1224): n is a, c, g, or t
  misc_feature (1243)..(1243): n is a, c, g, or t
  misc_feature (1245)..(1245): n is a, c, g, or t
  misc_feature (1248)..(1248): n is a, c, g, or t
  misc_feature (1252)..(1252): n is a, c, g, or t
  misc_feature (1298)..(1298): n is a, c, g, or t
  misc_feature (1301)..(1302): n is a, c, g, or t
  misc_feature (1317)..(1317): n is a, c, g, or t
  misc_feature (1332)..(1332): n is a, c, g, or t
  misc_feature (1379)..(1379): n is a, c, g, or t
  misc_feature (1413)..(1413): n is a, c, g, or t
  misc_feature (1416)..(1416): n is a, c, g, or t
  misc_feature (1420)..(1420): n is a, c, g, or t
  misc_feature (1422)..(1422): n is a, c, g, or t
  misc_feature (1431)..(1431): n is a, c, g, or t
  misc_feature (1433)..(1433): n is a, c, g, or t
  misc_feature (1446)..(1446): n is a, c, g, or t
  misc_feature (1488)..(1488): n is a, c, g, or t
  misc_feature (1500)..(1500): n is a, c, g, or t
  misc_feature (1507)..(1507): n is a, c, g, or t
  misc_feature (1514)..(1516): n is a, c, g, or t
  misc_feature (1531)..(1531): n is a, c, g, or t
  misc_feature (1533)..(1533): n is a, c, g, or t
  misc_feature (1546)..(1546): n is a, c, g, or t
  misc_feature (1549)..(1549): n is a, c, g, or t
  misc_feature (1567)..(1567): n is a, c, g, or t
  misc_feature (1575)..(1575): n is a, c, g, or t
  misc_feature (1578)..(1579): n is a, c, g, or t
  misc_feature (1586)..(1586): n is a, c, g, or t
  misc_feature (1594)..(1594): n is a, c, g, or t
  misc_feature (1596)..(1596): n is a, c, g, or t
  misc_feature (1604)..(1604): n is a, c, g, or t
  misc_feature (1606)..(1606): n is a, c, g, or t
  misc_feature (1629)..(1629): n is a, c, g, or t
  misc_feature (1637)..(1637): n is a, c, g, or t
  misc_feature (1656)..(1656): n is a, c, g, or t
  misc_feature (1674)..(1674): n is a, c, g, or t
  misc_feature (1677)..(1677): n is a, c, g, or t
  misc_feature (1685)..(1685): n is a, c, g, or t
  misc_feature (1715)..(1717): n is a, c, g, or t
  misc_feature (1723)..(1723): n is a, c, g, or t
  misc_feature (1757)..(1758): n is a, c, g, or t
  misc_feature (1764)..(1765): n is a, c, g, or t
  misc_feature (1776)..(1776): n is a, c, g, or t
  misc_feature (1784)..(1784): n is a, c, g, or t
  misc_feature (1789)..(1789): n is a, c, g, or t
  misc_feature (1799)..(1799): n is a, c, g, or t
  misc_feature (1812)..(1812): n is a, c, g, or t
  misc_feature (1816)..(1816): n is a, c, g, or t
  misc_feature (1832)..(1832): n is a, c, g, or t
  misc_feature (1835)..(1835): n is a, c, g, or t
  misc_feature (1847)..(1847): n is a, c, g, or t
  misc_feature (1860)..(1860): n is a, c, g, or t
  misc_feature (1866)..(1866): n is a, c, g, or t
  misc_feature (1871)..(1871): n is a, c, g, or t
  misc_feature (1874)..(1874): n is a, c, g, or t
  misc_feature (1888)..(1888): n is a, c, g, or t
  misc_feature (1901)..(1901): n is a, c, g, or t
  misc_feature (1908)..(1909): n is a, c, g, or t
  misc_feature (1911)..(1911): n is a, c, g, or t
  misc_feature (1918)..(1918): n is a, c, g, or t
  misc_feature (1924)..(1924): n is a, c, g, or t
  misc_feature (1946)..(1946): n is a, c, g, or t
  misc_feature (1951)..(1952): n is a, c, g, or t
  misc_feature (1964)..(1966): n is a, c, g, or t
  misc_feature (1976)..(1977): n is a, c, g, or t
  misc_feature (1999)..(1999): n is a, c, g, or t
  misc_feature (2003)..(2003): n is a, c, g, or t
  misc_feature (2008)..(2008): n is a, c, g, or t
  misc_feature (2020)..(2020): n is a, c, g, or t
  misc_feature (2037)..(2037): n is a, c, g, or t
  misc_feature (2052)..(2052): n is a, c, g, or t
  misc_feature (2064)..(2064): n is a, c, g, or t
  misc_feature (2079)..(2079): n is a, c, g, or t
  misc_feature (2088)..(2088): n is a, c, g, or t
  misc_feature (2092)..(2092): n is a, c, g, or t
  misc_feature (2103)..(2103): n is a, c, g, or t
  misc_feature (2109)..(2111): n is a, c, g, or t
  misc_feature (2120)..(2121): n is a, c, g, or t
  misc_feature (2123)..(2123): n is a, c, g, or t
  misc_feature (2125)..(2125): n is a, c, g, or t
  misc_feature (2128)..(2128): n is a, c, g, or t
  misc_feature (2131)..(2131): n is a, c, g, or t
  misc_feature (2133)..(2133): n is a, c, g, or t
  misc_feature (2135)..(2135): n is a, c, g, or t
  misc_feature (2143)..(2143): n is a, c, g, or t
  misc_feature (2155)..(2155): n is a, c, g, or t
  misc_feature (2165)..(2165): n is a, c, g, or t
  misc_feature (2172)..(2172): n is a, c, g, or t
  misc_feature (2177)..(2177): n is a, c, g, or t
  misc_feature (2190)..(2190): n is a, c, g, or t
  misc_feature (2245)..(2245): n is a, c, g, or t
  misc_feature (2257)..(2258): n is a, c, g, or t
  misc_feature (2263)..(2263): n is a, c, g, or t
  misc_feature (2267)..(2267): n is a, c, g, or t
  misc_feature (2278)..(2281): n is a, c, g, or t
  misc_feature (2291)..(2292): n is a, c, g, or t
  misc_feature (2296)..(2297): n is a, c, g, or t
  misc_feature (2310)..(2310): n is a, c, g, or t
  misc_feature (2356)..(2356): n is a, c, g, or t
  misc_feature (2392)..(2392): n is a, c, g, or t
  misc_feature (2402)..(2402): n is a, c, g, or t
  misc_feature (2406)..(2406): n is a, c, g, or t
  misc_feature (2408)..(2408): n is a, c, g, or t
  misc_feature (2415)..(2415): n is a, c, g, or t
  misc_feature (2417)..(2417): n is a, c, g, or t
  misc_feature (2419)..(2419): n is a, c, g, or t
  misc_feature (2421)..(2423): n is a, c, g, or t
atggcttcgactctgggcngcgatgngagnnncgagatagtgtttttcgatcttgagacngcngttncgaccnaatcgggncanccntntgcgattttggagtttggngctatcttagtttgccctatgaagctagnggagctctatagttacnccacnttggntcgaccnacnganctttcnctcatctncacgctcacgaagcgacgaagcggcattacgcgcgacggagttctctctgcacctacnttctctgaaatcgctgatgaantctacganattcnccncggtaagggtttctcnntttttttnnnnnnctnncncnntctctctnacncgaagntanaagtattgattttggtgtttctgtaggacgaatttgggnnggacataacataaagagattcgattgtgtaagantangagatgcatttgcagnaattggtntcnctccccnngagncnaaagntncaattgatncactttcgttnnngtctcagaagtttgggaagngagctggngacntgaaggtctctcttttttcgtcttcncgatgataaatctcaaagccnatagcttncttgttatcnttatagatatgaatttcnangtaacttcanagattcatcactcatcanagtngctaaaatttacnctnnnnnaanaangtagatggcntngcntgctacntatttcgngctaggagancangctcacaggtnaaannagtaaacgatacnntgtgccttttaacgattcnccagttgtntcaatatgggactaaacatggntangattcancaggagcttagatgatgtccggatgaatcttgaagtnntcaagnactgtncaaccntcttntttctggtattgntgtcttntcatttcttgaataatgatnaactcntaanttnaaaaggantagattanagnggttnngacatatctganttctgtctncngttntgnaaaagnnggntcnatcttccttncagaccacanctttgcaagccgtaaacatggnttgcaacttgcaagtatagtttgnnatatcactgagtttaagtacttggtgtttgcaggagtcnagtgtnccngacattcttananncatnagctggttntnccnaagaaaaagtcngngaacacgaagtaatgagaagtcactgccnnatggagtcagagaaagcccnacttcttcctcttcnagccctnaanctganccgagttcgtcttctgtanangcnacanctgtcaaaaaccatcccatcatttctcttctgacggaatgctcagnaanngatacatctagttgngaaatagatccatcngacataaccactctaataagtaaactacatattggaactcttaaganagatgctgcggacgaagccaaaactgtgagagangcngcgnangaagccaanantgtaagacagcanggtgaatcaaccgatcccaatgccaaagatgaatcatttttnggcgttaatgangtatctntttctannntcagggcaagtcttntnccgttatatcgtnggngtctgagaatggagctgnttcacaanganncccctcnacatctcngntggtatancntgaaaattcggtttggaataagncggaagtntgtggatcatgtaggtcgnccaaagatgaatattgtngtngacatacntcctgatttatgcaagatcttggacgcatnnnatgctnctgcgcataacttactgattgactcaagcacaanntcagannggaggcctacngttatgangaaanaaggctttgncaactatcccacngccngactgcagtaagtatncancactctctctgnccttttacatacnagcatnaatcnacnggagagtctctaanaccatctccaacnctactcnntnttcaccnccaaantctattttggagttaaatcccnccaanncttgcaaaatannnatcttcaaannttttctccatatttggagattntganttttnaagtcatgactncattttggagttgggtnggagaaaaacacaantccaaaataganttacttcattttggngtaaaaaantgangaaatgggttngagatnnnctaacctcnntnancantcntntnttgttggnagaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatgatctagccaaggaaaaatcattccnctgtctcttnctgntnagtcggngngnannn

SEQ ID NO: 23 - DNA, 1521 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (89)..(89): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (177)..(177): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (287)..(287): n is a, c, g, or t
  misc_feature (302)..(303): n is a, c, g, or t
  misc_feature (337)..(337): n is a, c, g, or t
  misc_feature (340)..(340): n is a, c, g, or t
  misc_feature (356)..(356): n is a, c, g, or t
  misc_feature (364)..(364): n is a, c, g, or t
  misc_feature (367)..(367): n is a, c, g, or t
  misc_feature (374)..(375): n is a, c, g, or t
  misc_feature (379)..(379): n is a, c, g, or t
  misc_feature (381)..(381): n is a, c, g, or t
  misc_feature (386)..(386): n is a, c, g, or t
  misc_feature (388)..(388): n is a, c, g, or t
  misc_feature (397)..(397): n is a, c, g, or t
  misc_feature (408)..(410): n is a, c, g, or t
  misc_feature (430)..(430): n is a, c, g, or t
  misc_feature (438)..(438): n is a, c, g, or t
  misc_feature (442)..(442): n is a, c, g, or t
  misc_feature (453)..(453): n is a, c, g, or t
  misc_feature (455)..(455): n is a, c, g, or t
  misc_feature (458)..(458): n is a, c, g, or t
  misc_feature (465)..(465): n is a, c, g, or t
  misc_feature (473)..(473): n is a, c, g, or t
  misc_feature (483)..(483): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (547)..(547): n is a, c, g, or t
  misc_feature (552)..(552): n is a, c, g, or t
  misc_feature (564)..(564): n is a, c, g, or t
  misc_feature (570)..(570): n is a, c, g, or t
  misc_feature (573)..(573): n is a, c, g, or t
  misc_feature (584)..(584): n is a, c, g, or t
  misc_feature (586)..(587): n is a, c, g, or t
  misc_feature (591)..(591): n is a, c, g, or t
  misc_feature (600)..(600): n is a, c, g, or t
  misc_feature (602)..(602): n is a, c, g, or t
  misc_feature (605)..(605): n is a, c, g, or t
  misc_feature (617)..(617): n is a, c, g, or t
  misc_feature (619)..(619): n is a, c, g, or t
  misc_feature (648)..(649): n is a, c, g, or t
  misc_feature (669)..(669): n is a, c, g, or t
  misc_feature (684)..(684): n is a, c, g, or t
  misc_feature (691)..(691): n is a, c, g, or t
  misc_feature (694)..(694): n is a, c, g, or t
  misc_feature (699)..(699): n is a, c, g, or t
  misc_feature (718)..(718): n is a, c, g, or t
  misc_feature (720)..(720): n is a, c, g, or t
  misc_feature (723)..(723): n is a, c, g, or t
  misc_feature (727)..(727): n is a, c, g, or t
  misc_feature (773)..(773): n is a, c, g, or t
  misc_feature (776)..(777): n is a, c, g, or t
  misc_feature (792)..(792): n is a, c, g, or t
  misc_feature (807)..(807): n is a, c, g, or t
  misc_feature (854)..(854): n is a, c, g, or t
  misc_feature (888)..(888): n is a, c, g, or t
  misc_feature (891)..(891): n is a, c, g, or t
  misc_feature (895)..(895): n is a, c, g, or t
  misc_feature (897)..(897): n is a, c, g, or t
  misc_feature (906)..(906): n is a, c, g, or t
  misc_feature (908)..(908): n is a, c, g, or t
  misc_feature (921)..(921): n is a, c, g, or t
  misc_feature (963)..(963): n is a, c, g, or t
  misc_feature (975)..(975): n is a, c, g, or t
  misc_feature (982)..(982): n is a, c, g, or t
  misc_feature (989)..(991): n is a, c, g, or t
  misc_feature (1006)..(1006): n is a, c, g, or t
  misc_feature (1008)..(1008): n is a, c, g, or t
  misc_feature (1021)..(1021): n is a, c, g, or t
  misc_feature (1024)..(1024): n is a, c, g, or t
  misc_feature (1042)..(1042): n is a, c, g, or t
  misc_feature (1050)..(1050): n is a, c, g, or t
  misc_feature (1053)..(1054): n is a, c, g, or t
  misc_feature (1061)..(1061): n is a, c, g, or t
  misc_feature (1069)..(1069): n is a, c, g, or t
  misc_feature (1071)..(1071): n is a, c, g, or t
  misc_feature (1079)..(1079): n is a, c, g, or t
  misc_feature (1081)..(1081): n is a, c, g, or t
  misc_feature (1104)..(1104): n is a, c, g, or t
  misc_feature (1112)..(1112): n is a, c, g, or t
  misc_feature (1131)..(1131): n is a, c, g, or t
  misc_feature (1149)..(1149): n is a, c, g, or t
  misc_feature (1152)..(1152): n is a, c, g, or t
  misc_feature (1160)..(1160): n is a, c, g, or t
  misc_feature (1190)..(1192): n is a, c, g, or t
  misc_feature (1198)..(1198): n is a, c, g, or t
  misc_feature (1232)..(1233): n is a, c, g, or t
  misc_feature (1239)..(1240): n is a, c, g, or t
  misc_feature (1251)..(1251): n is a, c, g, or t
  misc_feature (1259)..(1259): n is a, c, g, or t
  misc_feature (1264)..(1264): n is a, c, g, or t
  misc_feature (1274)..(1274): n is a, c, g, or t
  misc_feature (1287)..(1287): n is a, c, g, or t
  misc_feature (1291)..(1291): n is a, c, g, or t
  misc_feature (1308)..(1308): n is a, c, g, or t
  misc_feature (1318)..(1318): n is a, c, g, or t
  misc_feature (1325)..(1325): n is a, c, g, or t
  misc_feature (1330)..(1330): n is a, c, g, or t
  misc_feature (1343)..(1343): n is a, c, g, or t
  misc_feature (1398)..(1398): n is a, c, g, or t
  misc_feature (1410)..(1411): n is a, c, g, or t
  misc_feature (1416)..(1416): n is a, c, g, or t
  misc_feature (1420)..(1420): n is a, c, g, or t
  misc_feature (1431)..(1434): n is a, c, g, or t
  misc_feature (1444)..(1445): n is a, c, g, or t
  misc_feature (1449)..(1450): n is a, c, g, or t
  misc_feature (1463)..(1463): n is a, c, g, or t
  misc_feature (1509)..(1509): n is a, c, g, or t
atggcttcgactctgggcngcgatgngagnnncgagatagtgtttttcgatcttgagacngcngttncgaccnaatcgggncanccntntgcgattttggagtttggngctatcttagtttgccctatgaagctagnggagctctatagttacnccacnttggntcgaccnacnganctttcnctcatctncacgctcacgaagcgacgaagcggcattacgcgcgacggagttctctctgcacctacnttctctgaaatcgctgatgaantctacganattcnccncggacgaatttgggnnggacataacataaagagattcgattgtgtaagantangagatgcatttgcagnaattggtntcnctccccnngagncnaaagntncaattgatncactttcgttnnngtctcagaagtttgggaagngagctggngacntgaagatggcntngcntgctacntatttcgngctaggagancangctcacaggagcttagatgatgtccggatgaatcttgaagtnntcaagnactgtncaaccntcttntttctggagtcnagtgtnccngacattcttananncatnagctggttntnccnaagaaaaagtcngngaacacgaagtaatgagaagtcactgccnnatggagtcagagaaagcccnacttcttcctcttcnagccctnaanctganccgagttcgtcttctgtanangcnacanctgtcaaaaaccatcccatcatttctcttctgacggaatgctcagnaanngatacatctagttgngaaatagatccatcngacataaccactctaataagtaaactacatattggaactcttaaganagatgctgcggacgaagccaaaactgtgagagangcngcgnangaagccaanantgtaagacagcanggtgaatcaaccgatcccaatgccaaagatgaatcatttttnggcgttaatgangtatctntttctannntcagggcaagtcttntnccgttatatcgtnggngtctgagaatggagctgnttcacaanganncccctcnacatctcngntggtatancntgaaaattcggtttggaataagncggaagtntgtggatcatgtaggtcgnccaaagatgaatattgtngtngacatacntcctgatttatgcaagatcttggacgcatnnnatgctnctgcgcataacttactgattgactcaagcacaanntcagannggaggcctacngttatgangaaanaaggctttgncaactatcccacngccngactgcaaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatga

SEQ ID NO: 24 - DNA, 2393 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (89)..(89): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (177)..(177): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (287)..(287): n is a, c, g, or t
  misc_feature (303)..(304): n is a, c, g, or t
  misc_feature (312)..(317): n is a, c, g, or t
  misc_feature (320)..(321): n is a, c, g, or t
  misc_feature (323)..(323): n is a, c, g, or t
  misc_feature (325)..(326): n is a, c, g, or t
  misc_feature (334)..(334): n is a, c, g, or t
  misc_feature (337)..(337): n is a, c, g, or t
  misc_feature (343)..(343): n is a, c, g, or t
  misc_feature (346)..(346): n is a, c, g, or t
  misc_feature (385)..(386): n is a, c, g, or t
  misc_feature (420)..(420): n is a, c, g, or t
  misc_feature (423)..(423): n is a, c, g, or t
  misc_feature (439)..(439): n is a, c, g, or t
  misc_feature (447)..(447): n is a, c, g, or t
  misc_feature (450)..(450): n is a, c, g, or t
  misc_feature (457)..(458): n is a, c, g, or t
  misc_feature (462)..(462): n is a, c, g, or t
  misc_feature (464)..(464): n is a, c, g, or t
  misc_feature (469)..(469): n is a, c, g, or t
  misc_feature (471)..(471): n is a, c, g, or t
  misc_feature (480)..(480): n is a, c, g, or t
  misc_feature (491)..(493): n is a, c, g, or t
  misc_feature (513)..(513): n is a, c, g, or t
  misc_feature (521)..(521): n is a, c, g, or t
  misc_feature (525)..(525): n is a, c, g, or t
  misc_feature (551)..(551): n is a, c, g, or t
  misc_feature (572)..(572): n is a, c, g, or t
  misc_feature (580)..(580): n is a, c, g, or t
  misc_feature (590)..(590): n is a, c, g, or t
  misc_feature (608)..(608): n is a, c, g, or t
  misc_feature (610)..(610): n is a, c, g, or t
  misc_feature (620)..(620): n is a, c, g, or t
  misc_feature (638)..(638): n is a, c, g, or t
  misc_feature (642)..(642): n is a, c, g, or t
  misc_feature (655)..(655): n is a, c, g, or t
  misc_feature (658)..(662): n is a, c, g, or t
  misc_feature (665)..(665): n is a, c, g, or t
  misc_feature (668)..(668): n is a, c, g, or t
  misc_feature (678)..(678): n is a, c, g, or t
  misc_feature (680)..(680): n is a, c, g, or t
  misc_feature (683)..(683): n is a, c, g, or t
  misc_feature (690)..(690): n is a, c, g, or t
  misc_feature (698)..(698): n is a, c, g, or t
  misc_feature (708)..(708): n is a, c, g, or t
  misc_feature (711)..(711): n is a, c, g, or t
  misc_feature (722)..(722): n is a, c, g, or t
  misc_feature (726)..(727): n is a, c, g, or t
  misc_feature (740)..(741): n is a, c, g, or t
  misc_feature (760)..(760): n is a, c, g, or t
  misc_feature (769)..(769): n is a, c, g, or t
  misc_feature (791)..(791): n is a, c, g, or t
  misc_feature (794)..(794): n is a, c, g, or t
  misc_feature (801)..(801): n is a, c, g, or t
  misc_feature (838)..(839): n is a, c, g, or t
  misc_feature (845)..(845): n is a, c, g, or t
  misc_feature (851)..(851): n is a, c, g, or t
  misc_feature (857)..(857): n is a, c, g, or t
  misc_feature (862)..(862): n is a, c, g, or t
  misc_feature (875)..(875): n is a, c, g, or t
  misc_feature (882)..(882): n is a, c, g, or t
  misc_feature (902)..(902): n is a, c, g, or t
  misc_feature (908)..(908): n is a, c, g, or t
  misc_feature (912)..(912): n is a, c, g, or t
  misc_feature (915)..(915): n is a, c, g, or t
  misc_feature (923)..(923): n is a, c, g, or t
  misc_feature (931)..(931): n is a, c, g, or t
  misc_feature (934)..(934): n is a, c, g, or t
  misc_feature (939)..(940): n is a, c, g, or t
  misc_feature (952)..(952): n is a, c, g, or t
  misc_feature (961)..(961): n is a, c, g, or t
  misc_feature (963)..(963): n is a, c, g, or t
  misc_feature (967)..(967): n is a, c, g, or t
  misc_feature (970)..(970): n is a, c, g, or t
  misc_feature (976)..(977): n is a, c, g, or t
  misc_feature (980)..(980): n is a, c, g, or t
  misc_feature (983)..(983): n is a, c, g, or t
  misc_feature (993)..(993): n is a, c, g, or t
  misc_feature (1003)..(1003): n is a, c, g, or t
  misc_feature (1025)..(1025): n is a, c, g, or t
  misc_feature (1049)..(1050): n is a, c, g, or t
  misc_feature (1089)..(1089): n is a, c, g, or t
  misc_feature (1095)..(1095): n is a, c, g, or t
  misc_feature (1098)..(1098): n is a, c, g, or t
  misc_feature (1109)..(1109): n is a, c, g, or t
  misc_feature (1111)..(1112): n is a, c, g, or t
  misc_feature (1116)..(1116): n is a, c, g, or t
  misc_feature (1125)..(1125): n is a, c, g, or t
  misc_feature (1127)..(1127): n is a, c, g, or t
  misc_feature (1130)..(1130): n is a, c, g, or t
  misc_feature (1142)..(1142): n is a, c, g, or t
  misc_feature (1144)..(1144): n is a, c, g, or t
  misc_feature (1173)..(1174): n is a, c, g, or t
  misc_feature (1194)..(1194): n is a, c, g, or t
  misc_feature (1209)..(1209): n is a, c, g, or t
  misc_feature (1216)..(1216): n is a, c, g, or t
  misc_feature (1219)..(1219): n is a, c, g, or t
  misc_feature (1224)..(1224): n is a, c, g, or t
  misc_feature (1243)..(1243): n is a, c, g, or t
  misc_feature (1245)..(1245): n is a, c, g, or t
  misc_feature (1248)..(1248): n is a, c, g, or t
  misc_feature (1252)..(1252): n is a, c, g, or t
  misc_feature (1298)..(1298): n is a, c, g, or t
  misc_feature (1301)..(1302): n is a, c, g, or t
  misc_feature (1317)..(1317): n is a, c, g, or t
  misc_feature (1332)..(1332): n is a, c, g, or t
  misc_feature (1379)..(1379): n is a, c, g, or t
  misc_feature (1383)..(1383): n is a, c, g, or t
  misc_feature (1386)..(1386): n is a, c, g, or t
  misc_feature (1390)..(1390): n is a, c, g, or t
  misc_feature (1392)..(1392): n is a, c, g, or t
  misc_feature (1401)..(1401): n is a, c, g, or t
  misc_feature (1403)..(1403): n is a, c, g, or t
  misc_feature (1416)..(1416): n is a, c, g, or t
  misc_feature (1458)..(1458): n is a, c, g, or t
  misc_feature (1470)..(1470): n is a, c, g, or t
  misc_feature (1477)..(1477): n is a, c, g, or t
  misc_feature (1484)..(1486): n is a, c, g, or t
  misc_feature (1501)..(1501): n is a, c, g, or t
  misc_feature (1503)..(1503): n is a, c, g, or t
  misc_feature (1516)..(1516): n is a, c, g, or t
  misc_feature (1519)..(1519): n is a, c, g, or t
  misc_feature (1537)..(1537): n is a, c, g, or t
  misc_feature (1545)..(1545): n is a, c, g, or t
  misc_feature (1548)..(1549): n is a, c, g, or t
  misc_feature (1556)..(1556): n is a, c, g, or t
  misc_feature (1564)..(1564): n is a, c, g, or t
  misc_feature (1566)..(1566): n is a, c, g, or t
  misc_feature (1574)..(1574): n is a, c, g, or t
  misc_feature (1576)..(1576): n is a, c, g, or t
  misc_feature (1599)..(1599): n is a, c, g, or t
  misc_feature (1607)..(1607): n is a, c, g, or t
  misc_feature (1626)..(1626): n is a, c, g, or t
  misc_feature (1644)..(1644): n is a, c, g, or t
  misc_feature (1647)..(1647): n is a, c, g, or t
  misc_feature (1655)..(1655): n is a, c, g, or t
  misc_feature (1685)..(1687): n is a, c, g, or t
  misc_feature (1693)..(1693): n is a, c, g, or t
  misc_feature (1727)..(1728): n is a, c, g, or t
  misc_feature (1734)..(1735): n is a, c, g, or t
  misc_feature (1746)..(1746): n is a, c, g, or t
  misc_feature (1754)..(1754): n is a, c, g, or t
  misc_feature (1759)..(1759): n is a, c, g, or t
  misc_feature (1769)..(1769): n is a, c, g, or t
  misc_feature (1782)..(1782): n is a, c, g, or t
  misc_feature (1786)..(1786): n is a, c, g, or t
  misc_feature (1802)..(1802): n is a, c, g, or t
  misc_feature (1805)..(1805): n is a, c, g, or t
  misc_feature (1817)..(1817): n is a, c, g, or t
  misc_feature (1830)..(1830): n is a, c, g, or t
  misc_feature (1836)..(1836): n is a, c, g, or t
  misc_feature (1841)..(1841): n is a, c, g, or t
  misc_feature (1844)..(1844): n is a, c, g, or t
  misc_feature (1858)..(1858): n is a, c, g, or t
  misc_feature (1871)..(1871): n is a, c, g, or t
  misc_feature (1878)..(1879): n is a, c, g, or t
  misc_feature (1881)..(1881): n is a, c, g, or t
  misc_feature (1888)..(1888): n is a, c, g, or t
  misc_feature (1894)..(1894): n is a, c, g, or t
  misc_feature (1916)..(1916): n is a, c, g, or t
  misc_feature (1921)..(1922): n is a, c, g, or t
  misc_feature (1934)..(1936): n is a, c, g, or t
  misc_feature (1946)..(1947): n is a, c, g, or t
  misc_feature (1969)..(1969): n is a, c, g, or t
  misc_feature (1973)..(1973): n is a, c, g, or t
  misc_feature (1978)..(1978): n is a, c, g, or t
  misc_feature (1990)..(1990): n is a, c, g, or t
  misc_feature (2007)..(2007): n is a, c, g, or t
  misc_feature (2022)..(2022): n is a, c, g, or t
  misc_feature (2034)..(2034): n is a, c, g, or t
  misc_feature (2049)..(2049): n is a, c, g, or t
  misc_feature (2058)..(2058): n is a, c, g, or t
  misc_feature (2062)..(2062): n is a, c, g, or t
  misc_feature (2073)..(2073): n is a, c, g, or t
  misc_feature (2079)..(2081): n is a, c, g, or t
  misc_feature (2090)..(2091): n is a, c, g, or t
  misc_feature (2093)..(2093): n is a, c, g, or t
  misc_feature (2095)..(2095): n is a, c, g, or t
  misc_feature (2098)..(2098): n is a, c, g, or t
  misc_feature (2101)..(2101): n is a, c, g, or t
  misc_feature (2103)..(2103): n is a, c, g, or t
  misc_feature (2105)..(2105): n is a, c, g, or t
  misc_feature (2113)..(2113): n is a, c, g, or t
  misc_feature (2125)..(2125): n is a, c, g, or t
  misc_feature (2135)..(2135): n is a, c, g, or t
  misc_feature (2142)..(2142): n is a, c, g, or t
  misc_feature (2147)..(2147): n is a, c, g, or t
  misc_feature (2160)..(2160): n is a, c, g, or t
  misc_feature (2215)..(2215): n is a, c, g, or t
  misc_feature (2227)..(2228): n is a, c, g, or t
  misc_feature (2233)..(2233): n is a, c, g, or t
  misc_feature (2237)..(2237): n is a, c, g, or t
  misc_feature (2248)..(2251): n is a, c, g, or t
  misc_feature (2261)..(2262): n is a, c, g, or t
  misc_feature (2266)..(2267): n is a, c, g, or t
  misc_feature (2280)..(2280): n is a, c, g, or t
  misc_feature (2326)..(2326): n is a, c, g, or t
  misc_feature (2362)..(2362): n is a, c, g, or t
  misc_feature (2372)..(2372): n is a, c, g, or t
  misc_feature (2376)..(2376): n is a, c, g, or t
  misc_feature (2378)..(2378): n is a, c, g, or t
  misc_feature (2385)..(2385): n is a, c, g, or t
  misc_feature (2387)..(2387): n is a, c, g, or t
  misc_feature (2389)..(2389): n is a, c, g, or t
  misc_feature (2391)..(2393): n is a, c, g, or t
atggcttcgactctgggcngcgatgngagnnncgagatagtgtttttcgatcttgagacngcngttncgaccnaatcgggncanccntntgcgattttggagtttggngctatcttagtttgccctatgaagctagnggagctctatagttacnccacnttggntcgaccnacnganctttcnctcatctncacgctcacgaagcgacgaagcggcattacgcgcgacggagttctctctgcacctacnttctctgaaatcgctgatgaantctacganattcnccncggtaagggtttctcnntttttttnnnnnnctnncncnntctctctnacncgaagntanaagtattgattttggtgtttctgtaggacgaatttgggnnggacataacataaagagattcgattgtgtaagantangagatgcatttgcagnaattggtntcnctccccnngagncnaaagntncaattgatncactttcgttnnngtctcagaagtttgggaagngagctggngacntgaaggtctctcttttttcgtcttcncgatgataaatctcaaagccnatagcttncttgttatcnttatagatatgaatttcnangtaacttcanagattcatcactcatcanagtngctaaaatttacnctnnnnnaanaangtagatggcntngcntgctacntatttcgngctaggagancangctcacaggtnaaannagtaaacgatacnntgtgccttttaacgattcnccagttgtntcaatatgggactaaacatggntangattcancaggagcttagatgatgtccggatgaatcttgaagtnntcaagnactgtncaaccntcttntttctggtattgntgtcttntcatttcttgaataatgatnaactcntaanttnaaaaggantagattanagnggttnngacatatctganttctgtctncngttntgnaaaagnnggntcnatcttccttncagaccacanctttgcaagccgtaaacatggnttgcaacttgcaagtatagtttgnnatatcactgagtttaagtacttggtgtttgcaggagtcnagtgtnccngacattcttananncatnagctggttntnccnaagaaaaagtcngngaacacgaagtaatgagaagtcactgccnnatggagtcagagaaagcccnacttcttcctcttcnagccctnaanctganccgagttcgtcttctgtanangcnacanctgtcaaaaaccatcccatcatttctcttctgacggaatgctcagnaanngatacatctagttgngaaatagatccatcngacataaccactctaataagtaaactacatattggaactcttaaganagangcngcgnangaagccaanantgtaagacagcanggtgaatcaaccgatcccaatgccaaagatgaatcatttttnggcgttaatgangtatctntttctannntcagggcaagtcttntnccgttatatcgtnggngtctgagaatggagctgnttcacaanganncccctcnacatctcngntggtatancntgaaaattcggtttggaataagncggaagtntgtggatcatgtaggtcgnccaaagatgaatattgtngtngacatacntcctgatttatgcaagatcttggacgcatnnnatgctnctgcgcataacttactgattgactcaagcacaanntcagannggaggcctacngttatgangaaanaaggctttgncaactatcccacngccngactgcagtaagtatncancactctctctgnccttttacatacnagcatnaatcnacnggagagtctctaanaccatctccaacnctactcnntnttcaccnccaaantctattttggagttaaatcccnccaanncttgcaaaatannnatcttcaaannttttctccatatttggagattntganttttnaagtcatgactncattttggagttgggtnggagaaaaacacaantccaaaataganttacttcattttggngtaaaaaantgangaaatgggttngagatnnnctaacctcnntnancantcntntnttgttggnagaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatgatctagccaaggaaaaatcattccnctgtctcttnctgntnagtcggngngnannn

SEQ ID NO: 25 - DNA, 1491 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (89)..(89): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (177)..(177): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (287)..(287): n is a, c, g, or t
  misc_feature (302)..(303): n is a, c, g, or t
  misc_feature (337)..(337): n is a, c, g, or t
  misc_feature (340)..(340): n is a, c, g, or t
  misc_feature (356)..(356): n is a, c, g, or t
  misc_feature (364)..(364): n is a, c, g, or t
  misc_feature (367)..(367): n is a, c, g, or t
  misc_feature (374)..(375): n is a, c, g, or t
  misc_feature (379)..(379): n is a, c, g, or t
  misc_feature (381)..(381): n is a, c, g, or t
  misc_feature (386)..(386): n is a, c, g, or t
  misc_feature (388)..(388): n is a, c, g, or t
  misc_feature (397)..(397): n is a, c, g, or t
  misc_feature (408)..(410): n is a, c, g, or t
  misc_feature (430)..(430): n is a, c, g, or t
  misc_feature (438)..(438): n is a, c, g, or t
  misc_feature (442)..(442): n is a, c, g, or t
  misc_feature (453)..(453): n is a, c, g, or t
  misc_feature (455)..(455): n is a, c, g, or t
  misc_feature (458)..(458): n is a, c, g, or t
  misc_feature (465)..(465): n is a, c, g, or t
  misc_feature (473)..(473): n is a, c, g, or t
  misc_feature (483)..(483): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (547)..(547): n is a, c, g, or t
  misc_feature (552)..(552): n is a, c, g, or t
  misc_feature (564)..(564): n is a, c, g, or t
  misc_feature (570)..(570): n is a, c, g, or t
  misc_feature (573)..(573): n is a, c, g, or t
  misc_feature (584)..(584): n is a, c, g, or t
  misc_feature (586)..(587): n is a, c, g, or t
  misc_feature (591)..(591): n is a, c, g, or t
  misc_feature (600)..(600): n is a, c, g, or t
  misc_feature (602)..(602): n is a, c, g, or t
  misc_feature (605)..(605): n is a, c, g, or t
  misc_feature (617)..(617): n is a, c, g, or t
  misc_feature (619)..(619): n is a, c, g, or t
  misc_feature (648)..(649): n is a, c, g, or t
  misc_feature (669)..(669): n is a, c, g, or t
  misc_feature (684)..(684): n is a, c, g, or t
  misc_feature (691)..(691): n is a, c, g, or t
  misc_feature (694)..(694): n is a, c, g, or t
  misc_feature (699)..(699): n is a, c, g, or t
  misc_feature (718)..(718): n is a, c, g, or t
  misc_feature (720)..(720): n is a, c, g, or t
  misc_feature (723)..(723): n is a, c, g, or t
  misc_feature (727)..(727): n is a, c, g, or t
  misc_feature (773)..(773): n is a, c, g, or t
  misc_feature (776)..(777): n is a, c, g, or t
  misc_feature (792)..(792): n is a, c, g, or t
  misc_feature (807)..(807): n is a, c, g, or t
  misc_feature (854)..(854): n is a, c, g, or t
  misc_feature (858)..(858): n is a, c, g, or t
  misc_feature (861)..(861): n is a, c, g, or t
  misc_feature (865)..(865): n is a, c, g, or t
  misc_feature (867)..(867): n is a, c, g, or t
  misc_feature (876)..(876): n is a, c, g, or t
  misc_feature (878)..(878): n is a, c, g, or t
  misc_feature (891)..(891): n is a, c, g, or t
  misc_feature (933)..(933): n is a, c, g, or t
  misc_feature (945)..(945): n is a, c, g, or t
  misc_feature (952)..(952): n is a, c, g, or t
  misc_feature (959)..(961): n is a, c, g, or t
  misc_feature (976)..(976): n is a, c, g, or t
  misc_feature (978)..(978): n is a, c, g, or t
  misc_feature (991)..(991): n is a, c, g, or t
  misc_feature (994)..(994): n is a, c, g, or t
  misc_feature (1012)..(1012): n is a, c, g, or t
  misc_feature (1020)..(1020): n is a, c, g, or t
  misc_feature (1023)..(1024): n is a, c, g, or t
  misc_feature (1031)..(1031): n is a, c, g, or t
  misc_feature (1039)..(1039): n is a, c, g, or t
  misc_feature (1041)..(1041): n is a, c, g, or t
  misc_feature (1049)..(1049): n is a, c, g, or t
  misc_feature (1051)..(1051): n is a, c, g, or t
  misc_feature (1074)..(1074): n is a, c, g, or t
  misc_feature (1082)..(1082): n is a, c, g, or t
  misc_feature (1101)..(1101): n is a, c, g, or t
  misc_feature (1119)..(1119): n is a, c, g, or t
  misc_feature (1122)..(1122): n is a, c, g, or t
  misc_feature (1130)..(1130): n is a, c, g, or t
  misc_feature (1160)..(1162): n is a, c, g, or t
  misc_feature (1168)..(1168): n is a, c, g, or t
  misc_feature (1202)..(1203): n is a, c, g, or t
  misc_feature (1209)..(1210): n is a, c, g, or t
  misc_feature (1221)..(1221): n is a, c, g, or t
  misc_feature (1229)..(1229): n is a, c, g, or t
  misc_feature (1234)..(1234): n is a, c, g, or t
  misc_feature (1244)..(1244): n is a, c, g, or t
  misc_feature (1257)..(1257): n is a, c, g, or t
  misc_feature (1261)..(1261): n is a, c, g, or t
  misc_feature (1278)..(1278): n is a, c, g, or t
  misc_feature (1288)..(1288): n is a, c, g, or t
  misc_feature (1295)..(1295): n is a, c, g, or t
  misc_feature (1300)..(1300): n is a, c, g, or t
  misc_feature (1313)..(1313): n is a, c, g, or t
  misc_feature (1368)..(1368): n is a, c, g, or t
  misc_feature (1380)..(1381): n is a, c, g, or t
  misc_feature (1386)..(1386): n is a, c, g, or t
  misc_feature (1390)..(1390): n is a, c, g, or t
  misc_feature (1401)..(1404): n is a, c, g, or t
  misc_feature (1414)..(1415): n is a, c, g, or t
  misc_feature (1419)..(1420): n is a, c, g, or t
  misc_feature (1433)..(1433): n is a, c, g, or t
  misc_feature (1479)..(1479): n is a, c, g, or t
atggcttcgactctgggcngcgatgngagnnncgagatagtgtttttcgatcttgagacngcngttncgaccnaatcgggncanccntntgcgattttggagtttggngctatcttagtttgccctatgaagctagnggagctctatagttacnccacnttggntcgaccnacnganctttcnctcatctncacgctcacgaagcgacgaagcggcattacgcgcgacggagttctctctgcacctacnttctctgaaatcgctgatgaantctacganattcnccncggacgaatttgggnnggacataacataaagagattcgattgtgtaagantangagatgcatttgcagnaattggtntcnctccccnngagncnaaagntncaattgatncactttcgttnnngtctcagaagtttgggaagngagctggngacntgaagatggcntngcntgctacntatttcgngctaggagancangctcacaggagcttagatgatgtccggatgaatcttgaagtnntcaagnactgtncaaccntcttntttctggagtcnagtgtnccngacattcttananncatnagctggttntnccnaagaaaaagtcngngaacacgaagtaatgagaagtcactgccnnatggagtcagagaaagcccnacttcttcctcttcnagccctnaanctganccgagttcgtcttctgtanangcnacanctgtcaaaaaccatcccatcatttctcttctgacggaatgctcagnaanngatacatctagttgngaaatagatccatcngacataaccactctaataagtaaactacatattggaactcttaaganagangcngcgnangaagccaanantgtaagacagcanggtgaatcaaccgatcccaatgccaaagatgaatcatttttnggcgttaatgangtatctntttctannntcagggcaagtcttntnccgttatatcgtnggngtctgagaatggagctgnttcacaanganncccctcnacatctcngntggtatancntgaaaattcggtttggaataagncggaagtntgtggatcatgtaggtcgnccaaagatgaatattgtngtngacatacntcctgatttatgcaagatcttggacgcatnnnatgctnctgcgcataacttactgattgactcaagcacaanntcagannggaggcctacngttatgangaaanaaggctttgncaactatcccacngccngactgcaaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatga

SEQ ID NO: 26 - DNA, 495 bp, Boechera
  misc_feature (21)..(21): n is a, c, g, or t
  misc_feature (24)..(24): n is a, c, g, or t
  misc_feature (28)..(28): n is a, c, g, or t
  misc_feature (34)..(34): n is a, c, g, or t
  misc_feature (42)..(42): n is a, c, g, or t
  misc_feature (45)..(45): n is a, c, g, or t
  misc_feature (48)..(48): n is a, c, g, or t
  misc_feature (50)..(50): n is a, c, g, or t
  misc_feature (69)..(69): n is a, c, g, or t
  misc_feature (98)..(98): n is a, c, g, or t
  misc_feature (115)..(115): n is a, c, g, or t
  misc_feature (120)..(120): n is a, c, g, or t
  misc_feature (125)..(125): n is a, c, g, or t
  misc_feature (132)..(132): n is a, c, g, or t
  misc_feature (135)..(135): n is a, c, g, or t
  misc_feature (138)..(138): n is a, c, g, or t
  misc_feature (144)..(144): n is a, c, g, or t
  misc_feature (152)..(152): n is a, c, g, or t
  misc_feature (210)..(210): n is a, c, g, or t
  misc_feature (232)..(232): n is a, c, g, or t
  misc_feature (240)..(240): n is a, c, g, or t
  misc_feature (245)..(245): n is a, c, g, or t
  misc_feature (248)..(248): n is a, c, g, or t
  misc_feature (263)..(264): n is a, c, g, or t
  misc_feature (298)..(298): n is a, c, g, or t
  misc_feature (301)..(301): n is a, c, g, or t
  misc_feature (317)..(317): n is a, c, g, or t
  misc_feature (325)..(325): n is a, c, g, or t
  misc_feature (328)..(328): n is a, c, g, or t
  misc_feature (335)..(336): n is a, c, g, or t
  misc_feature (340)..(340): n is a, c, g, or t
  misc_feature (342)..(342): n is a, c, g, or t
  misc_feature (347)..(347): n is a, c, g, or t
  misc_feature (349)..(349): n is a, c, g, or t
  misc_feature (358)..(358): n is a, c, g, or t
  misc_feature (369)..(371): n is a, c, g, or t
  misc_feature (391)..(391): n is a, c, g, or t
  misc_feature (399)..(399): n is a, c, g, or t
  misc_feature (403)..(403): n is a, c, g, or t
  misc_feature (414)..(414): n is a, c, g, or t
  misc_feature (416)..(416): n is a, c, g, or t
  misc_feature (419)..(419): n is a, c, g, or t
  misc_feature (426)..(426): n is a, c, g, or t
  misc_feature (434)..(434): n is a, c, g, or t
  misc_feature (444)..(444): n is a, c, g, or t
  misc_feature (447)..(447): n is a, c, g, or t
  misc_feature (489)..(490): n is a, c, g, or t
gtgtttttcgatcttgagacngcngttncgaccnaatcgggncanccntntgcgattttggagtttggngctatcttagtttgccctatgaagctagnggagctctatagttacnccacnttggntcgaccnacnganctttcnctcatctncacgctcacgaagcgacgaagcggcattacgcgcgacggagttctctctgcacctacnttctctgaaatcgctgatgaantctacganattcnccncggacgaatttgggnnggacataacataaagagattcgattgtgtaagantangagatgcatttgcagnaattggtntcnctccccnngagncnaaagntncaattgatncactttcgttnnngtctcagaagtttgggaagngagctggngacntgaagatggcntngcntgctacntatttcgngctaggagancangctcacaggagcttagatgatgtccggatgaatcttgaagtnntcaag

SEQ ID NO: 27 - DNA, 2536 bp, Boechera
  misc_feature (107)..(107): n is a, c, g, or t
  misc_feature (141)..(141): n is a, c, g, or t
  misc_feature (175)..(175): n is a, c, g, or t
  misc_feature (199)..(199): n is a, c, g, or t
  misc_feature (204)..(204): n is a, c, g, or t
  misc_feature (274)..(274): n is a, c, g, or t
  misc_feature (286)..(286): n is a, c, g, or t
  misc_feature (292)..(292): n is a, c, g, or t
  misc_feature (402)..(402): n is a, c, g, or t
  misc_feature (427)..(432): n is a, c, g, or t
  misc_feature (435)..(436): n is a, c, g, or t
  misc_feature (440)..(440): n is a, c, g, or t
  misc_feature (449)..(449): n is a, c, g, or t
  misc_feature (452)..(452): n is a, c, g, or t
  misc_feature (458)..(458): n is a, c, g, or t
  misc_feature (500)..(501): n is a, c, g, or t
  misc_feature (538)..(538): n is a, c, g, or t
  misc_feature (554)..(554): n is a, c, g, or t
  misc_feature (565)..(565): n is a, c, g, or t
  misc_feature (606)..(607): n is a, c, g, or t
  misc_feature (664)..(664): n is a, c, g, or t
  misc_feature (687)..(687): n is a, c, g, or t
  misc_feature (695)..(695): n is a, c, g, or t
  misc_feature (723)..(723): n is a, c, g, or t
  misc_feature (735)..(735): n is a, c, g, or t
  misc_feature (773)..(777): n is a, c, g, or t
  misc_feature (780)..(780): n is a, c, g, or t
  misc_feature (783)..(783): n is a, c, g, or t
  misc_feature (798)..(798): n is a, c, g, or t
  misc_feature (812)..(812): n is a, c, g, or t
  misc_feature (826)..(826): n is a, c, g, or t
  misc_feature (837)..(837): n is a, c, g, or t
  misc_feature (841)..(842): n is a, c, g, or t
  misc_feature (856)..(856): n is a, c, g, or t
  misc_feature (906)..(906): n is a, c, g, or t
  misc_feature (909)..(909): n is a, c, g, or t
  misc_feature (953)..(954): n is a, c, g, or t
  misc_feature (960)..(960): n is a, c, g, or t
  misc_feature (966)..(966): n is a, c, g, or t
  misc_feature (977)..(977): n is a, c, g, or t
  misc_feature (990)..(990): n is a, c, g, or t
  misc_feature (997)..(997): n is a, c, g, or t
  misc_feature (1017)..(1017): n is a, c, g, or t
  misc_feature (1026)..(1026): n is a, c, g, or t
  misc_feature (1029)..(1029): n is a, c, g, or t
  misc_feature (1045)..(1045): n is a, c, g, or t
  misc_feature (1052)..(1052): n is a, c, g, or t
  misc_feature (1054)..(1054): n is a, c, g, or t
  misc_feature (1066)..(1066): n is a, c, g, or t
  misc_feature (1077)..(1077): n is a, c, g, or t
  misc_feature (1106)..(1106): n is a, c, g, or t
  misc_feature (1116)..(1116): n is a, c, g, or t
  misc_feature (1138)..(1138): n is a, c, g, or t
  misc_feature (1140)..(1145): n is a, c, g, or t
  misc_feature (1163)..(1163): n is a, c, g, or t
  misc_feature (1202)..(1202): n is a, c, g, or t
  misc_feature (1208)..(1208): n is a, c, g, or t
  misc_feature (1211)..(1211): n is a, c, g, or t
  misc_feature (1222)..(1222): n is a, c, g, or t
  misc_feature (1224)..(1225): n is a, c, g, or t
  misc_feature (1229)..(1229): n is a, c, g, or t
  misc_feature (1238)..(1238): n is a, c, g, or t
  misc_feature (1240)..(1240): n is a, c, g, or t
  misc_feature (1257)..(1257): n is a, c, g, or t
  misc_feature (1287)..(1287): n is a, c, g, or t
  misc_feature (1322)..(1322): n is a, c, g, or t
  misc_feature (1329)..(1329): n is a, c, g, or t
  misc_feature (1337)..(1337): n is a, c, g, or t
  misc_feature (1365)..(1365): n is a, c, g, or t
  misc_feature (1411)..(1411): n is a, c, g, or t
  misc_feature (1415)..(1415): n is a, c, g, or t
  misc_feature (1492)..(1492): n is a, c, g, or t
  misc_feature (1529)..(1529): n is a, c, g, or t
  misc_feature (1535)..(1535): n is a, c, g, or t
  misc_feature (1544)..(1544): n is a, c, g, or t
  misc_feature (1546)..(1546): n is a, c, g, or t
  misc_feature (1627)..(1627): n is a, c, g, or t
  misc_feature (1659)..(1659): n is a, c, g, or t
  misc_feature (1662)..(1662): n is a, c, g, or t
  misc_feature (1680)..(1680): n is a, c, g, or t
  misc_feature (1688)..(1688): n is a, c, g, or t
  misc_feature (1692)..(1692): n is a, c, g, or t
  misc_feature (1699)..(1699): n is a, c, g, or t
  misc_feature (1709)..(1709): n is a, c, g, or t
  misc_feature (1798)..(1798): n is a, c, g, or t
  misc_feature (1828)..(1828): n is a, c, g, or t
  misc_feature (1870)..(1871): n is a, c, g, or t
  misc_feature (1878)..(1878): n is a, c, g, or t
  misc_feature (1889)..(1889): n is a, c, g, or t
  misc_feature (1897)..(1897): n is a, c, g, or t
  misc_feature (1902)..(1902): n is a, c, g, or t
  misc_feature (1912)..(1912): n is a, c, g, or t
  misc_feature (1945)..(1945): n is a, c, g, or t
  misc_feature (1948)..(1948): n is a, c, g, or t
  misc_feature (2001)..(2001): n is a, c, g, or t
  misc_feature (2021)..(2022): n is a, c, g, or t
  misc_feature (2059)..(2059): n is a, c, g, or t
  misc_feature (2079)..(2079): n is a, c, g, or t
  misc_feature (2089)..(2090): n is a, c, g, or t
  misc_feature (2112)..(2112): n is a, c, g, or t
  misc_feature (2116)..(2116): n is a, c, g, or t
  misc_feature (2133)..(2133): n is a, c, g, or t
  misc_feature (2165)..(2165): n is a, c, g, or t
  misc_feature (2177)..(2177): n is a, c, g, or t
  misc_feature (2201)..(2201): n is a, c, g, or t
  misc_feature (2205)..(2205): n is a, c, g, or t
  misc_feature (2216)..(2216): n is a, c, g, or t
  misc_feature (2222)..(2224): n is a, c, g, or t
  misc_feature (2233)..(2234): n is a, c, g, or t
  misc_feature (2246)..(2246): n is a, c, g, or t
  misc_feature (2248)..(2248): n is a, c, g, or t
  misc_feature (2290)..(2290): n is a, c, g, or t
  misc_feature (2370)..(2370): n is a, c, g, or t
  misc_feature (2392)..(2394): n is a, c, g, or t
  misc_feature (2404)..(2405): n is a, c, g, or t
  misc_feature (2409)..(2410): n is a, c, g, or t
  misc_feature (2469)..(2469): n is a, c, g, or t
  misc_feature (2505)..(2505): n is a, c, g, or t
  misc_feature (2519)..(2519): n is a, c, g, or t
  misc_feature (2534)..(2536): n is a, c, g, or t
tcgtaccgttgcttctctcaagtttagatttttttccgtaaaaagaggaggtggcccgtgaagtttattccctttaaaacccaccaattagctccttcactctcagntctcaacaatggcttcgactctgggcggcgatgngagaaacgagatagtgtttttcgatcttgagacngcggttccgaccaaatcggggcanccttntgcgattttggagtttggggctatcttagtttgccctatgaagctagtggagctctatagttactccacnttggttcgaccnaccganctttctctcatctccacgctcacgaagcgacgaagcggcattacgcgcgacggagttctctctgcacctacattctctgaaatcgctgatgaagtctacgacattctccncggtaagggtttctctttttttttnnnnnnctnnctcnatctctctnacncgaagntacaagtattgattttggtgtttctgtaggacgaatttgggnnggacataacataaagagattcgattgtgtaagaatangagatgcatttgcagnaattggtctcnctcccccggagccgaaagctacaattgattcactttcgttnntgtctcagaagtttgggaagagagctggtgacatgaaggtctctcttttttcgtctnctcgatgataaatctcaaagccnatagcttncttgttatctttatagatatgaatttcnatgtaacttcanagattcatcactcatcaaagttgctaaaatttactctnnnnnaanaangtagatggcatcgcntgctacatatttcnggctaggagatcangctcacaggtnaaannagtaaacgataccntgtgccttttaacgattcaccagttgtttcaatatgggactaaacatggntangattcaccaggagcttagatgatgtccggatgaatcttgaagtnntcaagnactgtncaaccgtcttntttctggtattgntgtcttntcatttcttgaataatgatnaactctaanttnaaaaggattagattanagaggtngngacatatctganttctgtctacngtttgcaaaagttgggtccatcttccttncagaccacanctttgcaagccgtaaacatggntnnnnnnttgcaagtatagtttgtnatatcactgagtttaagtacttggtgtttgcaggagtcnagtgtnccngacattcttananncatnagctggttntncccaagaaaaagtccgngaacacgaagtaatgagaagtcactgcctnatggagtcagagaaagcccgacttcttcctcttcnagccctnaaactganccgagttcgtcttctgtagatgccacanctgtcaaaaaccatcccatcatttctcttctgacggaatgctcagnaagngatacatctagttgtgaaatagatccatctgacataaccactctaataagtaaactacatattggaactcttaaganagatgctgcggacgaagccaaaactgtgagagatgcngcggangaagccaanantgtaagacagcagggtgaatcaaccgatcccaatgccaaagatgaatcatttttgggcgttaatgaagtatctgtttctancatcagggcaagtcttatcccgttatatcgtnggngtctgagaatggagctgnttcacaangacncccctcnacatctctgntggtatagcttgaaaattcggtttggaataagccggaagtatgtggatcatgtaggtcgtccaaagatgaatattgttgtagacatacntcctgatttatgcaagatcttggacgcatncgatgctgctgcgcataacttactgattgactcaagcacaanntcagatnggaggcctacngttatgangaaanaaggctttgncaactatcccacagccagactgcagtaagtatncancactctctctgaccttttacatacgagcatgaatccaccggagagtctctaanaccatctccaaccctactcnntattcacctccaaactctattttggagttaaatcccnccaacccttgcaaaataganatcttcaaannttttctccatatttggagattntgantttttaagtcatgactncattttggagttgggttggagaaaaacacaantccaaaataganttacttcattttggagtaaaaaantgangaaatgggttngagatnnnctaacctcnntcaccattcttntnttgttggcagaataagctcagaatccaatggaacccaggtanaccaaaaagaagaacctttgggaaccaatcaaaagctcgatttcagtagcgataattttgaaaagcttgagtcagcactncttcctggtaccctggttgatnnnttcttctcanncganncttacgattataagaaaatggtagggatacgtctagcagccagaaagttggtaatccanctgaagaaatgatctagccaaggaaaaatcattccnctgtctcttcctgntcagtcggtgagcannn

SEQ ID NO: 28 - DNA, 1521 bp, Boechera
  misc_feature (26)..(26): n is a, c, g, or t
  misc_feature (60)..(60): n is a, c, g, or t
  misc_feature (84)..(84): n is a, c, g, or t
  misc_feature (89)..(89): n is a, c, g, or t
  misc_feature (159)..(159): n is a, c, g, or t
  misc_feature (171)..(171): n is a, c, g, or t
  misc_feature (177)..(177): n is a, c, g, or t
  misc_feature (287)..(287): n is a, c, g, or t
  misc_feature (302)..(303): n is a, c, g, or t
  misc_feature (340)..(340): n is a, c, g, or t
  misc_feature (356)..(356): n is a, c, g, or t
  misc_feature (367)..(367): n is a, c, g, or t
  misc_feature (408)..(409): n is a, c, g, or t
  misc_feature (458)..(458): n is a, c, g, or t
  misc_feature (472)..(472): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (552)..(552): n is a, c, g, or t
  misc_feature (564)..(564): n is a, c, g, or t
  misc_feature (570)..(570): n is a, c, g, or t
  misc_feature (573)..(573): n is a, c, g, or t
  misc_feature (584)..(584): n is a, c, g, or t
  misc_feature (586)..(587): n is a, c, g, or t
  misc_feature (591)..(591): n is a, c, g, or t
  misc_feature (600)..(600): n is a, c, g, or t
  misc_feature (602)..(602): n is a, c, g, or t
  misc_feature (619)..(619): n is a, c, g, or t
  misc_feature (649)..(649): n is a, c, g, or t
  misc_feature (684)..(684): n is a, c, g, or t
  misc_feature (691)..(691): n is a, c, g, or t
  misc_feature (699)..(699): n is a, c, g, or t
  misc_feature (727)..(727): n is a, c, g, or t
  misc_feature (773)..(773): n is a, c, g, or t
  misc_feature (777)..(777): n is a, c, g, or t
  misc_feature (854)..(854): n is a, c, g, or t
  misc_feature (891)..(891): n is a, c, g, or t
  misc_feature (897)..(897): n is a, c, g, or t
  misc_feature (906)..(906): n is a, c, g, or t
  misc_feature (908)..(908): n is a, c, g, or t
  misc_feature (989)..(989): n is a, c, g, or t
  misc_feature (1021)..(1021): n is a, c, g, or t
  misc_feature (1024)..(1024): n is a, c, g, or t
  misc_feature (1042)..(1042): n is a, c, g, or t
  misc_feature (1050)..(1050): n is a, c, g, or t
  misc_feature (1054)..(1054): n is a, c, g, or t
  misc_feature (1061)..(1061): n is a, c, g, or t
  misc_feature (1071)..(1071): n is a, c, g, or t
  misc_feature (1160)..(1160): n is a, c, g, or t
  misc_feature (1190)..(1190): n is a, c, g, or t
  misc_feature (1232)..(1233): n is a, c, g, or t
  misc_feature (1240)..(1240): n is a, c, g, or t
  misc_feature (1251)..(1251): n is a, c, g, or t
  misc_feature (1259)..(1259): n is a, c, g, or t
  misc_feature (1264)..(1264): n is a, c, g, or t
  misc_feature (1274)..(1274): n is a, c, g, or t
  misc_feature (1330)..(1330): n is a, c, g, or t
  misc_feature (1410)..(1410): n is a, c, g, or t
  misc_feature (1432)..(1434): n is a, c, g, or t
  misc_feature (1444)..(1445): n is a, c, g, or t
  misc_feature (1449)..(1450): n is a, c, g, or t
  misc_feature (1509)..(1509): n is a, c, g, or t
atggcttcgactctgggcggcgatgngagaaacgagatagtgtttttcgatcttgagacngcggttccgaccaaatcggggcanccttntgcgattttggagtttggggctatcttagtttgccctatgaagctagtggagctctatagttactccacnttggttcgaccnaccganctttctctcatctccacgctcacgaagcgacgaagcggcattacgcgcgacggagttctctctgcacctacattctctgaaatcgctgatgaagtctacgacattctccncggacgaatttgggnnggacataacataaagagattcgattgtgtaagaatangagatgcatttgcagnaattggtctcnctcccccggagccgaaagctacaattgattcactttcgttnntgtctcagaagtttgggaagagagctggtgacatgaagatggcatcgcntgctacatatttcnggctaggagatcangctcacaggagcttagatgatgtccggatgaatcttgaagtnntcaagnactgtncaaccgtcttntttctggagtcnagtgtnccngacattcttananncatnagctggttntncccaagaaaaagtccgngaacacgaagtaatgagaagtcactgcctnatggagtcagagaaagcccgacttcttcctcttcnagccctnaaactganccgagttcgtcttctgtagatgccacanctgtcaaaaaccatcccatcatttctcttctgacggaatgctcagnaagngatacatctagttgtgaaatagatccatctgacataaccactctaataagtaaactacatattggaactcttaaganagatgctgcggacgaagccaaaactgtgagagatgcngcggangaagccaanantgtaagacagcagggtgaatcaaccgatcccaatgccaaagatgaatcatttttgggcgttaatgaagtatctgtttctancatcagggcaagtcttatcccgttatatcgtnggngtctgagaatggagctgnttcacaangacncccctcnacatctctgntggtatagcttgaaaattcggtttggaataagccggaagtatgtggatcatgtaggtcgtccaaagatgaatattgttgtagacatacntcctgatttatgcaagatcttggacgcatncgatgctgctgcgcataacttactgattgactcaagcacaanntcagatnggaggcctacngttatgangaaanaaggctttgncaactatcccacagccagactgcaaataagctcagaatccaatggaacccaggtanaccaaaaagaagaacctttgggaaccaatcaaaagctcgatttcagtagcgataattttgaaaagcttgagtcagcactncttcctggtaccctggttgatnnnttcttctcanncganncttacgattataagaaaatggtagggatacgtctagcagccagaaagttggtaatccanctgaagaaatga

SEQ ID NO: 29 - DNA, 2506 bp, Boechera
  misc_feature (107)..(107): n is a, c, g, or t
  misc_feature (141)..(141): n is a, c, g, or t
  misc_feature (175)..(175): n is a, c, g, or t
  misc_feature (199)..(199): n is a, c, g, or t
  misc_feature (204)..(204): n is a, c, g, or t
  misc_feature (274)..(274): n is a, c, g, or t
  misc_feature (286)..(286): n is a, c, g, or t
  misc_feature (292)..(292): n is a, c, g, or t
  misc_feature (402)..(402): n is a, c, g, or t
  misc_feature (427)..(432): n is a, c, g, or t
  misc_feature (435)..(436): n is a, c, g, or t
  misc_feature (440)..(440): n is a, c, g, or t
  misc_feature (449)..(449): n is a, c, g, or t
  misc_feature (452)..(452): n is a, c, g, or t
  misc_feature (458)..(458): n is a, c, g, or t
  misc_feature (500)..(501): n is a, c, g, or t
  misc_feature (538)..(538): n is a, c, g, or t
  misc_feature (554)..(554): n is a, c, g, or t
  misc_feature (565)..(565): n is a, c, g, or t
  misc_feature (606)..(607): n is a, c, g, or t
  misc_feature (664)..(664): n is a, c, g, or t
  misc_feature (687)..(687): n is a, c, g, or t
  misc_feature (695)..(695): n is a, c, g, or t
  misc_feature (723)..(723): n is a, c, g, or t
  misc_feature (735)..(735): n is a, c, g, or t
  misc_feature (773)..(777): n is a, c, g, or t
  misc_feature (780)..(780): n is a, c, g, or t
  misc_feature (783)..(783): n is a, c, g, or t
  misc_feature (798)..(798): n is a, c, g, or t
  misc_feature (812)..(812): n is a, c, g, or t
  misc_feature (826)..(826): n is a, c, g, or t
  misc_feature (837)..(837): n is a, c, g, or t
  misc_feature (841)..(842): n is a, c, g, or t
  misc_feature (856)..(856): n is a, c, g, or t
  misc_feature (906)..(906): n is a, c, g, or t
  misc_feature (909)..(909): n is a, c, g, or t
  misc_feature (953)..(954): n is a, c, g, or t
  misc_feature (960)..(960): n is a, c, g, or t
  misc_feature (966)..(966): n is a, c, g, or t
  misc_feature (977)..(977): n is a, c, g, or t
  misc_feature (990)..(990): n is a, c, g, or t
  misc_feature (997)..(997): n is a, c, g, or t
  misc_feature (1017)..(1017): n is a, c, g, or t
  misc_feature (1026)..(1026): n is a, c, g, or t
  misc_feature (1029)..(1029): n is a, c, g, or t
  misc_feature (1045)..(1045): n is a, c, g, or t
  misc_feature (1052)..(1052): n is a, c, g, or t
  misc_feature (1054)..(1054): n is a, c, g, or t
  misc_feature (1066)..(1066): n is a, c, g, or t
  misc_feature (1077)..(1077): n is a, c, g, or t
  misc_feature (1106)..(1106): n is a, c, g, or t
  misc_feature (1116)..(1116): n is a, c, g, or t
  misc_feature (1138)..(1138): n is a, c, g, or t
  misc_feature (1140)..(1145): n is a, c, g, or t
  misc_feature (1163)..(1163): n is a, c, g, or t
  misc_feature (1202)..(1202): n is a, c, g, or t
  misc_feature (1208)..(1208): n is a, c, g, or t
  misc_feature (1211)..(1211): n is a, c, g, or t
  misc_feature (1222)..(1222): n is a, c, g, or t
  misc_feature (1224)..(1225): n is a, c, g, or t
  misc_feature (1229)..(1229): n is a, c, g, or t
  misc_feature (1238)..(1238): n is a, c, g, or t
  misc_feature (1240)..(1240): n is a, c, g, or t
  misc_feature (1257)..(1257): n is a, c, g, or t
  misc_feature (1287)..(1287): n is a, c, g, or t
  misc_feature (1322)..(1322): n is a, c, g, or t
  misc_feature (1329)..(1329): n is a, c, g, or t
  misc_feature (1337)..(1337): n is a, c, g, or t
  misc_feature (1365)..(1365): n is a, c, g, or t
  misc_feature (1411)..(1411): n is a, c, g, or t
  misc_feature (1415)..(1415): n is a, c, g, or t
  misc_feature (1492)..(1492): n is a, c, g, or t
  misc_feature (1499)..(1499): n is a, c, g, or t
  misc_feature (1505)..(1505): n is a, c, g, or t
  misc_feature (1514)..(1514): n is a, c, g, or t
  misc_feature (1516)..(1516): n is a, c, g, or t
  misc_feature (1597)..(1597): n is a, c, g, or t
  misc_feature (1629)..(1629): n is a, c, g, or t
  misc_feature (1632)..(1632): n is a, c, g, or t
  misc_feature (1650)..(1650): n is a, c, g, or t
  misc_feature (1658)..(1658): n is a, c, g, or t
  misc_feature (1662)..(1662): n is a, c, g, or t
  misc_feature (1669)..(1669): n is a, c, g, or t
  misc_feature (1679)..(1679): n is a, c, g, or t
  misc_feature (1768)..(1768): n is a, c, g, or t
  misc_feature (1798)..(1798): n is a, c, g, or t
  misc_feature (1840)..(1841): n is a, c, g, or t
  misc_feature (1848)..(1848): n is a, c, g, or t
  misc_feature (1859)..(1859): n is a, c, g, or t
  misc_feature (1867)..(1867): n is a, c, g, or t
  misc_feature (1872)..(1872): n is a, c, g, or t
  misc_feature (1882)..(1882): n is a, c, g, or t
  misc_feature (1915)..(1915): n is a, c, g, or t
  misc_feature (1918)..(1918): n is a, c, g, or t
  misc_feature (1971)..(1971): n is a, c, g, or t
  misc_feature (1991)..(1992): n is a, c, g, or t
  misc_feature (2029)..(2029): n is a, c, g, or t
  misc_feature (2049)..(2049): n is a, c, g, or t
  misc_feature (2059)..(2060): n is a, c, g, or t
  misc_feature (2082)..(2082): n is a, c, g, or t
  misc_feature (2086)..(2086): n is a, c, g, or t
  misc_feature (2103)..(2103): n is a, c, g, or t
  misc_feature (2135)..(2135): n is a, c, g, or t
  misc_feature (2147)..(2147): n is a, c, g, or t
  misc_feature (2171)..(2171): n is a, c, g, or t
  misc_feature (2175)..(2175): n is a, c, g, or t
  misc_feature (2186)..(2186): n is a, c, g, or t
  misc_feature (2192)..(2194): n is a, c, g, or t
  misc_feature (2203)..(2204): n is a, c, g, or t
  misc_feature (2216)..(2216): n is a, c, g, or t
  misc_feature (2218)..(2218): n is a, c, g, or t
  misc_feature (2260)..(2260): n is a, c, g, or t
  misc_feature (2340)..(2340): n is a, c, g, or t
  misc_feature (2362)..(2364): n is a, c, g, or t
  misc_feature (2374)..(2375): n is a, c, g, or t
  misc_feature (2379)..(2380): n is a, c, g, or t
  misc_feature (2439)..(2439): n is a, c, g, or t
  misc_feature (2475)..(2475): n is a, c, g, or t
  misc_feature (2489)..(2489): n is a, c, g, or t
  misc_feature (2504)..(2506): n is a, c, g, or t
tcgtaccgttgcttctctcaagtttagatttttttccgtaaaaagaggaggtggcccgtgaagtttattccctttaaaacccaccaattagctccttcactctcagntctcaacaatggcttcgactctgggcggcgatgngagaaacgagatagtgtttttcgatcttgagacngcggttccgaccaaatcggggcanccttntgcgattttggagtttggggctatcttagtttgccctatgaagctagtggagctctatagttactccacnttggttcgaccnaccganctttctctcatctccacgctcacgaagcgacgaagcggcattacgcgcgacggagttctctctgcacctacattctctgaaatcgctgatgaagtctacgacattctccncggtaagggtttctctttttttttnnnnnnctnnctcnatctctctnacncgaagntacaagtattgattttggtgtttctgtaggacgaatttgggnnggacataacataaagagattcgattgtgtaagaatangagatgcatttgcagnaattggtctcnctcccccggagccgaaagctacaattgattcactttcgttnntgtctcagaagtttgggaagagagctggtgacatgaaggtctctcttttttcgtctnctcgatgataaatctcaaagccnatagcttncttgttatctttatagatatgaatttcnatgtaacttcanagattcatcactcatcaaagttgctaaaatttactctnnnnnaanaangtagatggcatcgcntgctacatatttcnggctaggagatcangctcacaggtnaaannagtaaacgataccntgtgccttttaacgattcaccagttgtttcaatatgggactaaacatggntangattcaccaggagcttagatgatgtccggatgaatcttgaagtnntcaagnactgtncaaccgtcttntttctggtattgntgtcttntcatttcttgaataatgatnaactctaanttnaaaaggattagattanagaggtngngacatatctganttctgtctacngtttgcaaaagttgggtccatcttccttncagaccacanctttgcaagccgtaaacatggntnnnnnnttgcaagtatagtttgtnatatcactgagtttaagtacttggtgtttgcaggagtcnagtgtnccngacattcttananncatnagctggttntncccaagaaaaagtccgngaacacgaagtaatgagaagtcactgcctnatggagtcagagaaagcccgacttcttcctcttcnagccctnaaactganccgagttcgtcttctgtagatgccacanctgtcaaaaaccatcccatcatttctcttctgacggaatgctcagnaagngatacatctagttgtgaaatagatccatctgacataaccactctaataagtaaactacatattggaactcttaaganagatgcngcggangaagccaanantgtaagacagcagggtgaatcaaccgatcccaatgccaaagatgaatcatttttgggcgttaatgaagtatctgtttctancatcagggcaagtcttatcccgttatatcgtnggngtctgagaatggagctgnttcacaangacncccctcnacatctctgntggtatagcttgaaaattcggtttggaataagccggaagtatgtggatcatgtaggtcgtccaaagatgaatattgttgtagacatacntcctgatttatgcaagatcttggacgcatncgatgctgctgcgcataacttactgattgactcaagcacaanntcagatnggaggcctacngttatgangaaanaaggctttgncaactatcccacagccagactgcagtaagtatncancactctctctgaccttttacatacgagcatgaatccaccggagagtctctaanaccatctccaaccctactcnntattcacctccaaactctattttggagttaaatcccnccaacccttgcaaaataganatcttcaaannttttctccatatttggagattntgantttttaagtcatgactncattttggagttgggttggagaaaaacacaantccaaaataganttacttcattttggagtaaaaaantgangaaatgggttngagatnnnctaacctcnntcaccattcttntnttgttggcagaataagctcagaatccaatggaacccaggtanaccaaaaagaagaacctttgggaaccaatcaaaagctcgatttcagtagcgataattttgaaaagcttgagtcagcactncttcctggtaccctggttgatnnnttcttctcanncganncttacgattataagaaaatggtagggatacgtctagcagccagaaagttggtaatccanctgaagaaatgatctagccaaggaaaaatcattccnctgtctcttcctgntcagtcggtgagcannn

SEQ ID NO: 30 - DNA, 1491 bp, Boechera
  misc_feature (26)..(26): n is a, c, g, or t
  misc_feature (60)..(60): n is a, c, g, or t
  misc_feature (84)..(84): n is a, c, g, or t
  misc_feature (89)..(89): n is a, c, g, or t
  misc_feature (159)..(159): n is a, c, g, or t
  misc_feature (171)..(171): n is a, c, g, or t
  misc_feature (177)..(177): n is a, c, g, or t
  misc_feature (287)..(287): n is a, c, g, or t
  misc_feature (302)..(303): n is a, c, g, or t
  misc_feature (340)..(340): n is a, c, g, or t
  misc_feature (356)..(356): n is a, c, g, or t
  misc_feature (367)..(367): n is a, c, g, or t
  misc_feature (408)..(409): n is a, c, g, or t
  misc_feature (458)..(458): n is a, c, g, or t
  misc_feature (472)..(472): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (552)..(552): n is a, c, g, or t
  misc_feature (564)..(564): n is a, c, g, or t
  misc_feature (570)..(570): n is a, c, g, or t
  misc_feature (573)..(573): n is a, c, g, or t
  misc_feature (584)..(584): n is a, c, g, or t
  misc_feature (586)..(587): n is a, c, g, or t
  misc_feature (591)..(591): n is a, c, g, or t
  misc_feature (600)..(600): n is a, c, g, or t
  misc_feature (602)..(602): n is a, c, g, or t
  misc_feature (619)..(619): n is a, c, g, or t
  misc_feature (649)..(649): n is a, c, g, or t
  misc_feature (684)..(684): n is a, c, g, or t
  misc_feature (691)..(691): n is a, c, g, or t
  misc_feature (699)..(699): n is a, c, g, or t
  misc_feature (727)..(727): n is a, c, g, or t
  misc_feature (773)..(773): n is a, c, g, or t
  misc_feature (777)..(777): n is a, c, g, or t
  misc_feature (854)..(854): n is a, c, g, or t
  misc_feature (861)..(861): n is a, c, g, or t
  misc_feature (867)..(867): n is a, c, g, or t
  misc_feature (876)..(876): n is a, c, g, or t
  misc_feature (878)..(878): n is a, c, g, or t
  misc_feature (959)..(959): n is a, c, g, or t
  misc_feature (991)..(991): n is a, c, g, or t
  misc_feature (994)..(994): n is a, c, g, or t
  misc_feature (1012)..(1012): n is a, c, g, or t
  misc_feature (1020)..(1020): n is a, c, g, or t
  misc_feature (1024)..(1024): n is a, c, g, or t
  misc_feature (1031)..(1031): n is a, c, g, or t
  misc_feature (1041)..(1041): n is a, c, g, or t
  misc_feature (1130)..(1130): n is a, c, g, or t
  misc_feature (1160)..(1160): n is a, c, g, or t
  misc_feature (1202)..(1203): n is a, c, g, or t
  misc_feature (1210)..(1210): n is a, c, g, or t
  misc_feature (1221)..(1221): n is a, c, g, or t
  misc_feature (1229)..(1229): n is a, c, g, or t
  misc_feature (1234)..(1234): n is a, c, g, or t
  misc_feature (1244)..(1244): n is a, c, g, or t
  misc_feature (1300)..(1300): n is a, c, g, or t
  misc_feature (1380)..(1380): n is a, c, g, or t
  misc_feature (1402)..(1404): n is a, c, g, or t
  misc_feature (1414)..(1415): n is a, c, g, or t
  misc_feature (1419)..(1420): n is a, c, g, or t
  misc_feature (1479)..(1479): n is a, c, g, or t
atggcttcgactctgggcggcgatgngagaaacgagatagtgtttttcgatcttgagacngcggttccgaccaaatcggggcanccttntgcgattttggagtttggggctatcttagtttgccctatgaagctagtggagctctatagttactccacnttggttcgaccnaccganctttctctcatctccacgctcacgaagcgacgaagcggcattacgcgcgacggagttctctctgcacctacattctctgaaatcgctgatgaagtctacgacattctccncggacgaatttgggnnggacataacataaagagattcgattgtgtaagaatangagatgcatttgcagnaattggtctcnctcccccggagccgaaagctacaattgattcactttcgttnntgtctcagaagtttgggaagagagctggtgacatgaagatggcatcgcntgctacatatttcnggctaggagatcangctcacaggagcttagatgatgtccggatgaatcttgaagtnntcaagnactgtncaaccgtcttntttctggagtcnagtgtnccngacattcttananncatnagctggttntncccaagaaaaagtccgngaacacgaagtaatgagaagtcactgcctnatggagtcagagaaagcccgacttcttcctcttcnagccctnaaactganccgagttcgtcttctgtagatgccacanctgtcaaaaaccatcccatcatttctcttctgacggaatgctcagnaagngatacatctagttgtgaaatagatccatctgacataaccactctaataagtaaactacatattggaactcttaaganagatgcngcggangaagccaanantgtaagacagcagggtgaatcaaccgatcccaatgccaaagatgaatcatttttgggcgttaatgaagtatctgtttctancatcagggcaagtcttatcccgttatatcgtnggngtctgagaatggagctgnttcacaangacncccctcnacatctctgntggtatagcttgaaaattcggtttggaataagccggaagtatgtggatcatgtaggtcgtccaaagatgaatattgttgtagacatacntcctgatttatgcaagatcttggacgcatncgatgctgctgcgcataacttactgattgactcaagcacaanntcagatnggaggcctacngttatgangaaanaaggctttgncaactatcccacagccagactgcaaataagctcagaatccaatggaacccaggtanaccaaaaagaagaacctttgggaaccaatcaaaagctcgatttcagtagcgataattttgaaaagcttgagtcagcactncttcctggtaccctggttgatnnnttcttctcanncganncttacgattataagaaaatggtagggatacgtctagcagccagaaagttggtaatccanctgaagaaatga

SEQ ID NO: 31 - DNA, 495 bp, Boechera
  misc_feature (21)..(21): n is a, c, g, or t
  misc_feature (45)..(45): n is a, c, g, or t
  misc_feature (50)..(50): n is a, c, g, or t
  misc_feature (120)..(120): n is a, c, g, or t
  misc_feature (132)..(132): n is a, c, g, or t
  misc_feature (138)..(138): n is a, c, g, or t
  misc_feature (248)..(248): n is a, c, g, or t
  misc_feature (263)..(264): n is a, c, g, or t
  misc_feature (301)..(301): n is a, c, g, or t
  misc_feature (317)..(317): n is a, c, g, or t
  misc_feature (328)..(328): n is a, c, g, or t
  misc_feature (369)..(370): n is a, c, g, or t
  misc_feature (419)..(419): n is a, c, g, or t
  misc_feature (433)..(433): n is a, c, g, or t
  misc_feature (447)..(447): n is a, c, g, or t
  misc_feature (489)..(490): n is a, c, g, or t
gtgtttttcgatcttgagacngcggttccgaccaaatcggggcanccttntgcgattttggagtttggggctatcttagtttgccctatgaagctagtggagctctatagttactccacnttggttcgaccnaccganctttctctcatctccacgctcacgaagcgacgaagcggcattacgcgcgacggagttctctctgcacctacattctctgaaatcgctgatgaagtctacgacattctccncggacgaatttgggnnggacataacataaagagattcgattgtgtaagaatangagatgcatttgcagnaattggtctcnctcccccggagccgaaagctacaattgattcactttcgttnntgtctcagaagtttgggaagagagctggtgacatgaagatggcatcgcntgctacatatttcnggctaggagatcangctcacaggagcttagatgatgtccggatgaatcttgaagtnntcaag

SEQ ID NO: 32 - DNA, 2528 bp, Boechera
  misc_feature (1)..(1): n is a, c, g, or t
  misc_feature (5)..(6): n is a, c, g, or t
  misc_feature (9)..(9): n is a, c, g, or t
  misc_feature (13)..(13): n is a, c, g, or t
  misc_feature (23)..(23): n is a, c, g, or t
  misc_feature (32)..(32): n is a, c, g, or t
  misc_feature (36)..(38): n is a, c, g, or t
  misc_feature (45)..(45): n is a, c, g, or t
  misc_feature (55)..(55): n is a, c, g, or t
  misc_feature (57)..(57): n is a, c, g, or t
  misc_feature (69)..(69): n is a, c, g, or t
  misc_feature (97)..(98): n is a, c, g, or t
  misc_feature (101)..(101): n is a, c, g, or t
  misc_feature (124)..(124): n is a, c, g, or t
  misc_feature (131)..(131): n is a, c, g, or t
  misc_feature (135)..(137): n is a, c, g, or t
  misc_feature (165)..(165): n is a, c, g, or t
  misc_feature (168)..(168): n is a, c, g, or t
  misc_feature (172)..(172): n is a, c, g, or t
  misc_feature (178)..(178): n is a, c, g, or t
  misc_feature (186)..(186): n is a, c, g, or t
  misc_feature (192)..(192): n is a, c, g, or t
  misc_feature (213)..(213): n is a, c, g, or t
  misc_feature (242)..(242): n is a, c, g, or t
  misc_feature (259)..(259): n is a, c, g, or t
  misc_feature (264)..(264): n is a, c, g, or t
  misc_feature (269)..(269): n is a, c, g, or t
  misc_feature (276)..(276): n is a, c, g, or t
  misc_feature (279)..(279): n is a, c, g, or t
  misc_feature (288)..(288): n is a, c, g, or t
  misc_feature (296)..(296): n is a, c, g, or t
  misc_feature (354)..(354): n is a, c, g, or t
  misc_feature (376)..(376): n is a, c, g, or t
  misc_feature (384)..(384): n is a, c, g, or t
  misc_feature (389)..(389): n is a, c, g, or t
  misc_feature (408)..(409): n is a, c, g, or t
  misc_feature (417)..(422): n is a, c, g, or t
  misc_feature (425)..(426): n is a, c, g, or t
  misc_feature (428)..(428): n is a, c, g, or t
  misc_feature (430)..(431): n is a, c, g, or t
  misc_feature (439)..(439): n is a, c, g, or t
  misc_feature (442)..(442): n is a, c, g, or t
  misc_feature (448)..(448): n is a, c, g, or t
  misc_feature (451)..(451): n is a, c, g, or t
  misc_feature (491)..(491): n is a, c, g, or t
  misc_feature (525)..(525): n is a, c, g, or t
  misc_feature (528)..(528): n is a, c, g, or t
  misc_feature (544)..(544): n is a, c, g, or t
  misc_feature (552)..(552): n is a, c, g, or t
  misc_feature (555)..(555): n is a, c, g, or t
  misc_feature (562)..(563): n is a, c, g, or t
  misc_feature (567)..(567): n is a, c, g, or t
  misc_feature (569)..(569): n is a, c, g, or t
  misc_feature (574)..(574): n is a, c, g, or t
  misc_feature (576)..(576): n is a, c, g, or t
  misc_feature (585)..(585): n is a, c, g, or t
  misc_feature (596)..(596): n is a, c, g, or t
  misc_feature (598)..(598): n is a, c, g, or t
  misc_feature (618)..(618): n is a, c, g, or t
  misc_feature (626)..(626): n is a, c, g, or t
  misc_feature (630)..(630): n is a, c, g, or t
  misc_feature (656)..(656): n is a, c, g, or t
  misc_feature (677)..(677): n is a, c, g, or t
  misc_feature (695)..(695): n is a, c, g, or t
  misc_feature (713)..(713): n is a, c, g, or t
  misc_feature (715)..(715): n is a, c, g, or t
  misc_feature (743)..(743): n is a, c, g, or t
  misc_feature (747)..(747): n is a, c, g, or t
  misc_feature (760)..(760): n is a, c, g, or t
  misc_feature (763)..(767): n is a, c, g, or t
  misc_feature (770)..(770): n is a, c, g, or t
  misc_feature (773)..(773): n is a, c, g, or t
  misc_feature (783)..(783): n is a, c, g, or t
  misc_feature (785)..(785): n is a, c, g, or t
  misc_feature (788)..(788): n is a, c, g, or t
  misc_feature (795)..(795): n is a, c, g, or t
  misc_feature (803)..(803): n is a, c, g, or t
  misc_feature (813)..(813): n is a, c, g, or t
  misc_feature (816)..(816): n is a, c, g, or t
  misc_feature (831)..(832): n is a, c, g, or t
  misc_feature (845)..(846): n is a, c, g, or t
  misc_feature (865)..(865): n is a, c, g, or t
  misc_feature (874)..(874): n is a, c, g, or t
  misc_feature (896)..(896): n is a, c, g, or t
  misc_feature (906)..(906): n is a, c, g, or t
  misc_feature (943)..(944): n is a, c, g, or t
  misc_feature (950)..(950): n is a, c, g, or t
  misc_feature (956)..(956): n is a, c, g, or t
  misc_feature (962)..(962): n is a, c, g, or t
  misc_feature (967)..(967): n is a, c, g, or t
  misc_feature (980)..(980): n is a, c, g, or t
  misc_feature (987)..(987): n is a, c, g, or t
  misc_feature (1007)..(1007): n is a, c, g, or t
  misc_feature (1013)..(1013): n is a, c, g, or t
  misc_feature (1017)..(1017): n is a, c, g, or t
  misc_feature (1020)..(1020): n is a, c, g, or t
  misc_feature (1028)..(1028): n is a, c, g, or t
  misc_feature (1036)..(1036): n is a, c, g, or t
  misc_feature (1039)..(1039): n is a, c, g, or t
  misc_feature (1044)..(1045): n is a, c, g, or t
  misc_feature (1057)..(1057): n is a, c, g, or t
  misc_feature (1066)..(1066): n is a, c, g, or t
  misc_feature (1072)..(1072): n is a, c, g, or t
  misc_feature (1075)..(1075): n is a, c, g, or t
  misc_feature (1081)..(1082): n is a, c, g, or t
  misc_feature (1085)..(1085): n is a, c, g, or t
  misc_feature (1088)..(1088): n is a, c, g, or t
  misc_feature (1098)..(1098): n is a, c, g, or t
  misc_feature (1130)..(1130): n is a, c, g, or t
  misc_feature (1154)..(1154): n is a, c, g, or t
  misc_feature (1214)..(1214): n is a, c, g, or t
  misc_feature (1216)..(1217): n is a, c, g, or t
  misc_feature (1230)..(1230): n is a, c, g, or t
  misc_feature (1232)..(1232): n is a, c, g, or t
  misc_feature (1235)..(1235): n is a, c, g, or t
  misc_feature (1247)..(1247): n is a, c, g, or t
  misc_feature (1278)..(1278): n is a, c, g, or t
  misc_feature (1299)..(1299): n is a, c, g, or t
  misc_feature (1314)..(1314): n is a, c, g, or t
  misc_feature (1324)..(1324): n is a, c, g, or t
  misc_feature (1348)..(1348): n is a, c, g, or t
  misc_feature (1350)..(1350): n is a, c, g, or t
  misc_feature (1353)..(1353): n is a, c, g, or t
  misc_feature (1357)..(1357): n is a, c, g, or t
  misc_feature (1406)..(1407): n is a, c, g, or t
  misc_feature (1422)..(1422): n is a, c, g, or t
  misc_feature (1437)..(1437): n is a, c, g, or t
  misc_feature (1484)..(1484): n is a, c, g, or t
  misc_feature (1518)..(1518): n is a, c, g, or t
  misc_feature (1525)..(1525): n is a, c, g, or t
  misc_feature (1527)..(1527): n is a, c, g, or t
  misc_feature (1538)..(1538): n is a, c, g, or t
  misc_feature (1551)..(1551): n is a, c, g, or t
  misc_feature (1593)..(1593): n is a, c, g, or t
  misc_feature (1605)..(1605): n is a, c, g, or t
  misc_feature (1612)..(1612): n is a, c, g, or t
  misc_feature (1620)..(1621): n is a, c, g, or t
  misc_feature (1636)..(1636): n is a, c, g, or t
  misc_feature (1638)..(1638): n is a, c, g, or t
  misc_feature (1651)..(1651): n is a, c, g, or t
  misc_feature (1654)..(1654): n is a, c, g, or t
  misc_feature (1672)..(1672): n is a, c, g, or t
  misc_feature (1680)..(1680): n is a, c, g, or t
  misc_feature (1683)..(1683): n is a, c, g, or t
  misc_feature (1699)..(1699): n is a, c, g, or t
  misc_feature (1701)..(1701): n is a, c, g, or t
  misc_feature (1709)..(1709): n is a, c, g, or t
  misc_feature (1711)..(1711): n is a, c, g, or t
  misc_feature (1734)..(1734): n is a, c, g, or t
  misc_feature (1742)..(1742): n is a, c, g, or t
  misc_feature (1761)..(1761): n is a, c, g, or t
  misc_feature (1779)..(1779): n is a, c, g, or t
  misc_feature (1782)..(1782): n is a, c, g, or t
  misc_feature (1820)..(1822): n is a, c, g, or t
  misc_feature (1828)..(1828): n is a, c, g, or t
  misc_feature (1862)..(1863): n is a, c, g, or t
  misc_feature (1869)..(1870): n is a, c, g, or t
  misc_feature (1881)..(1881): n is a, c, g, or t
  misc_feature (1889)..(1889): n is a, c, g, or t
  misc_feature (1894)..(1894): n is a, c, g, or t
  misc_feature (1917)..(1917): n is a, c, g, or t
  misc_feature (1921)..(1921): n is a, c, g, or t
  misc_feature (1940)..(1940): n is a, c, g, or t
  misc_feature (1952)..(1952): n is a, c, g, or t
  misc_feature (1965)..(1965): n is a, c, g, or t
  misc_feature (1971)..(1971): n is a, c, g, or t
  misc_feature (1976)..(1976): n is a, c, g, or t
  misc_feature (1979)..(1979): n is a, c, g, or t
  misc_feature (1993)..(1993): n is a, c, g, or t
  misc_feature (2006)..(2006): n is a, c, g, or t
  misc_feature (2013)..(2014): n is a, c, g, or t
  misc_feature (2016)..(2016): n is a, c, g, or t
  misc_feature (2023)..(2023): n is a, c, g, or t
  misc_feature (2029)..(2029): n is a, c, g, or t
  misc_feature (2056)..(2057): n is a, c, g, or t
  misc_feature (2069)..(2071): n is a, c, g, or t
  misc_feature (2081)..(2082): n is a, c, g, or t
  misc_feature (2108)..(2108): n is a, c, g, or t
  misc_feature (2113)..(2113): n is a, c, g, or t
  misc_feature (2142)..(2142): n is a, c, g, or t
  misc_feature (2157)..(2157): n is a, c, g, or t
  misc_feature (2169)..(2169): n is a, c, g, or t
  misc_feature (2184)..(2184): n is a, c, g, or t
  misc_feature (2193)..(2193): n is a, c, g, or t
  misc_feature (2208)..(2208): n is a, c, g, or t
  misc_feature (2214)..(2216): n is a, c, g, or t
  misc_feature (2225)..(2226): n is a, c, g, or t
  misc_feature (2228)..(2228): n is a, c, g, or t
  misc_feature (2230)..(2230): n is a, c, g, or t
  misc_feature (2233)..(2233): n is a, c, g, or t
  misc_feature (2236)..(2236): n is a, c, g, or t
  misc_feature (2238)..(2238): n is a, c, g, or t
  misc_feature (2240)..(2240): n is a, c, g, or t
  misc_feature (2248)..(2248): n is a, c, g, or t
  misc_feature (2260)..(2260): n is a, c, g, or t
  misc_feature (2270)..(2270): n is a, c, g, or t
  misc_feature (2277)..(2277): n is a, c, g, or t
  misc_feature (2282)..(2282): n is a, c, g, or t
  misc_feature (2295)..(2295): n is a, c, g, or t
  misc_feature (2350)..(2350): n is a, c, g, or t
  misc_feature (2362)..(2363): n is a, c, g, or t
  misc_feature (2368)..(2368): n is a, c, g, or t
  misc_feature (2372)..(2372): n is a, c, g, or t
  misc_feature (2383)..(2386): n is a, c, g, or t
  misc_feature (2396)..(2397): n is a, c, g, or t
  misc_feature (2401)..(2402): n is a, c, g, or t
  misc_feature (2415)..(2415): n is a, c, g, or t
  misc_feature (2461)..(2461): n is a, c, g, or t
  misc_feature (2507)..(2507): n is a, c, g, or t
  misc_feature (2513)..(2513): n is a, c, g, or t
  misc_feature (2520)..(2520): n is a, c, g, or t
  misc_feature (2522)..(2522): n is a, c, g, or t
  misc_feature (2524)..(2524): n is a, c, g, or t
ncgtnncgntgcntctctcaagnttagatttntttnnncgtaaanagaggaggancnattgctttaaancccaccaattagctccttcactctcagnnctnaacaatggcttcgactctgggcngcgatgngagnnncgagatagtgtttttcgatcttgagacngcngttncgaccnaatcgggncagccntttgcgattttggagtttggngctatcttagtttgccctatgaagctagnggagctctatagttacnccacnttggntcgaccnacngatctttcnctcatctncacgctcacgaagcgacgaagcggcattacgcgcgacggagttctctctgcacctacnttctctgaaatcgctgatgaantctacganattcnccacggtaagggtttctcnntttttttnnnnnnctnncncnntctctctnacncgaagntanaagtattgattttggtgtttctgtaggacgaatttgggcnggacataacataaagagattcgattgtgtaagantangagatgcatttgcagnaattggtntcnctccccnngagncnaaagntncaattgatncactttcgttntngtctcagaagtttgggaagngagctggngacntgaaggtctctcttttttcgtcttcncgatgataaatctcaaagccnatagcttccttgttatcnttatagatatgaatttcnangtaacttcaaagattcatcactcatcanagtngctaaaatttacnctnnnnnaanaangtagatggcntngcntgctacntatttcgngctaggagancangctcacaggtaaaannagtaaacgatacnntgtgccttttaacgattcnccagttgtntcaatatgggactaaacatggntatgattcancaggagcttagatgatgtccggatgaatcttgaagtnntcaagnactgtncaaccntcttntttctggtattgntgtcttntcatttcttgaataatgatnaactcntaanttnaaaaggantagattanagnggttnngacatatctganttctgtctncagttntgnaaaagnnggntcnatcttccttncagaccacaactttgcaagccgtaaacatggnttgcaacttgcaagtatagtttgntatatcactgagtttaagtacttggtgtttgcaggagtccagtgttcctgacattcttananncatgagctggttntnccnaagaaaaagtcngagaacacgaagtaatgagaagtcactgccnaatggagtcagagaaagcccnacttcttcctcttcnagccctaaanctgatccgagttcgtcttctgtanangcnacanctgtcaaaaaccatcccatcatttctcttctgacggaatgctcagaaanngatacatctagttgngaaatagatccatcngacataaccactctaataagtaaactacatattggaactcttaaganagatgctgcggacgaagccaaaactgtgagagangctgcgnangaagccaaaantgtaagacagcanggtgaatcaaccgatcccaatgccaaagatgaatcatttttnggcgttaatgangtatctntttctagnntcagggcaagtcttntnccgttatatcgtnggngtctgagaatggagctgnttcacaanganacccctctacatctcngntggtatancntgaaaattcggtttggaataagncggaagtntgtggatcatgtaggtcgnccaaagatgaatattgtngtngacatacctcctgatttatgcaagatcttggacgcatnnnatgctnctgcgcataacttactgattgactcaagcacaanntcagannggaggcctacngttatgangaaanaaggctttgccaactatcccacngccngactgcagtaagtattcancactctctctgnccttttacatacnagcatnaatcnacnggagagtctctaanaccatctccaacnctactcnntnttcaccnccaaantctattttggagttaaatccctccaanncttgcaaaatannnatcttcaaannttttctccatatttggagattttganttttnaagtcatgactccattttggagttgggtnggagaaaaacacaantccaaaataganttacttcattttggngtaaaaaantgaagaaatgggttngagatnnnctaacctcnntnancantcntntnttgttggnagaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatgatctagccaaggaaaaatcattcctctgtctcttnctggtnagtcggngngnactt

SEQ ID NO: 33 - DNA, 1521 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (87)..(87): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (303)..(303): n is a, c, g, or t
  misc_feature (337)..(337): n is a, c, g, or t
  misc_feature (340)..(340): n is a, c, g, or t
  misc_feature (356)..(356): n is a, c, g, or t
  misc_feature (364)..(364): n is a, c, g, or t
  misc_feature (367)..(367): n is a, c, g, or t
  misc_feature (374)..(375): n is a, c, g, or t
  misc_feature (379)..(379): n is a, c, g, or t
  misc_feature (381)..(381): n is a, c, g, or t
  misc_feature (386)..(386): n is a, c, g, or t
  misc_feature (388)..(388): n is a, c, g, or t
  misc_feature (397)..(397): n is a, c, g, or t
  misc_feature (408)..(408): n is a, c, g, or t
  misc_feature (410)..(410): n is a, c, g, or t
  misc_feature (430)..(430): n is a, c, g, or t
  misc_feature (438)..(438): n is a, c, g, or t
  misc_feature (442)..(442): n is a, c, g, or t
  misc_feature (453)..(453): n is a, c, g, or t
  misc_feature (455)..(455): n is a, c, g, or t
  misc_feature (458)..(458): n is a, c, g, or t
  misc_feature (465)..(465): n is a, c, g, or t
  misc_feature (473)..(473): n is a, c, g, or t
  misc_feature (483)..(483): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (547)..(547): n is a, c, g, or t
  misc_feature (552)..(552): n is a, c, g, or t
  misc_feature (584)..(584): n is a, c, g, or t
  misc_feature (586)..(587): n is a, c, g, or t
  misc_feature (600)..(600): n is a, c, g, or t
  misc_feature (602)..(602): n is a, c, g, or t
  misc_feature (605)..(605): n is a, c, g, or t
  misc_feature (617)..(617): n is a, c, g, or t
  misc_feature (648)..(648): n is a, c, g, or t
  misc_feature (669)..(669): n is a, c, g, or t
  misc_feature (684)..(684): n is a, c, g, or t
  misc_feature (694)..(694): n is a, c, g, or t
  misc_feature (718)..(718): n is a, c, g, or t
  misc_feature (720)..(720): n is a, c, g, or t
  misc_feature (723)..(723): n is a, c, g, or t
  misc_feature (727)..(727): n is a, c, g, or t
  misc_feature (776)..(777): n is a, c, g, or t
  misc_feature (792)..(792): n is a, c, g, or t
  misc_feature (807)..(807): n is a, c, g, or t
  misc_feature (854)..(854): n is a, c, g, or t
  misc_feature (888)..(888): n is a, c, g, or t
  misc_feature (895)..(895): n is a, c, g, or t
  misc_feature (897)..(897): n is a, c, g, or t
  misc_feature (908)..(908): n is a, c, g, or t
  misc_feature (921)..(921): n is a, c, g, or t
  misc_feature (963)..(963): n is a, c, g, or t
  misc_feature (975)..(975): n is a, c, g, or t
  misc_feature (982)..(982): n is a, c, g, or t
  misc_feature (990)..(991): n is a, c, g, or t
  misc_feature (1006)..(1006): n is a, c, g, or t
  misc_feature (1008)..(1008): n is a, c, g, or t
  misc_feature (1021)..(1021): n is a, c, g, or t
  misc_feature (1024)..(1024): n is a, c, g, or t
  misc_feature (1042)..(1042): n is a, c, g, or t
  misc_feature (1050)..(1050): n is a, c, g, or t
  misc_feature (1053)..(1053): n is a, c, g, or t
  misc_feature (1069)..(1069): n is a, c, g, or t
  misc_feature (1071)..(1071): n is a, c, g, or t
  misc_feature (1079)..(1079): n is a, c, g, or t
  misc_feature (1081)..(1081): n is a, c, g, or t
  misc_feature (1104)..(1104): n is a, c, g, or t
  misc_feature (1112)..(1112): n is a, c, g, or t
  misc_feature (1131)..(1131): n is a, c, g, or t
  misc_feature (1149)..(1149): n is a, c, g, or t
  misc_feature (1152)..(1152): n is a, c, g, or t
  misc_feature (1190)..(1192): n is a, c, g, or t
  misc_feature (1198)..(1198): n is a, c, g, or t
  misc_feature (1232)..(1233): n is a, c, g, or t
  misc_feature (1239)..(1240): n is a, c, g, or t
  misc_feature (1251)..(1251): n is a, c, g, or t
  misc_feature (1259)..(1259): n is a, c, g, or t
  misc_feature (1264)..(1264): n is a, c, g, or t
  misc_feature (1287)..(1287): n is a, c, g, or t
  misc_feature (1291)..(1291): n is a, c, g, or t
  misc_feature (1308)..(1308): n is a, c, g, or t
  misc_feature (1318)..(1318): n is a, c, g, or t
  misc_feature (1325)..(1325): n is a, c, g, or t
  misc_feature (1330)..(1330): n is a, c, g, or t
  misc_feature (1343)..(1343): n is a, c, g, or t
  misc_feature (1398)..(1398): n is a, c, g, or t
  misc_feature (1410)..(1411): n is a, c, g, or t
  misc_feature (1416)..(1416): n is a, c, g, or t
  misc_feature (1420)..(1420): n is a, c, g, or t
  misc_feature (1431)..(1434): n is a, c, g, or t
  misc_feature (1444)..(1445): n is a, c, g, or t
  misc_feature (1449)..(1450): n is a, c, g, or t
  misc_feature (1463)..(1463): n is a, c, g, or t
  misc_feature (1509)..(1509): n is a, c, g, or t
atggcttcgactctgggcngcgatgngagnnncgagatagtgtttttcgatcttgagacngcngttncgaccnaatcgggncagccntttgcgattttggagtttggngctatcttagtttgccctatgaagctagnggagctctatagttacnccacnttggntcgaccnacngatctttcnctcatctncacgctcacgaagcgacgaagcggcattacgcgcgacggagttctctctgcacctacnttctctgaaatcgctgatgaantctacganattcnccacggacgaatttgggcnggacataacataaagagattcgattgtgtaagantangagatgcatttgcagnaattggtntcnctccccnngagncnaaagntncaattgatncactttcgttntngtctcagaagtttgggaagngagctggngacntgaagatggcntngcntgctacntatttcgngctaggagancangctcacaggagcttagatgatgtccggatgaatcttgaagtnntcaagnactgtncaaccntcttntttctggagtccagtgttcctgacattcttananncatgagctggttntnccnaagaaaaagtcngagaacacgaagtaatgagaagtcactgccnaatggagtcagagaaagcccnacttcttcctcttcnagccctaaanctgatccgagttcgtcttctgtanangcnacanctgtcaaaaaccatcccatcatttctcttctgacggaatgctcagaaanngatacatctagttgngaaatagatccatcngacataaccactctaataagtaaactacatattggaactcttaaganagatgctgcggacgaagccaaaactgtgagagangctgcgnangaagccaaaantgtaagacagcanggtgaatcaaccgatcccaatgccaaagatgaatcatttttnggcgttaatgangtatctntttctagnntcagggcaagtcttntnccgttatatcgtnggngtctgagaatggagctgnttcacaanganacccctctacatctcngntggtatancntgaaaattcggtttggaataagncggaagtntgtggatcatgtaggtcgnccaaagatgaatattgtngtngacatacctcctgatttatgcaagatcttggacgcatnnnatgctnctgcgcataacttactgattgactcaagcacaanntcagannggaggcctacngttatgangaaanaaggctttgccaactatcccacngccngactgcaaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatga

SEQ ID NO: 34 - DNA, 2498 bp, Boechera
  misc_feature (1)..(1): n is a, c, g, or t
  misc_feature (5)..(6): n is a, c, g, or t
  misc_feature (9)..(9): n is a, c, g, or t
  misc_feature (13)..(13): n is a, c, g, or t
  misc_feature (23)..(23): n is a, c, g, or t
  misc_feature (32)..(32): n is a, c, g, or t
  misc_feature (36)..(38): n is a, c, g, or t
  misc_feature (45)..(45): n is a, c, g, or t
  misc_feature (55)..(55): n is a, c, g, or t
  misc_feature (57)..(57): n is a, c, g, or t
  misc_feature (69)..(69): n is a, c, g, or t
  misc_feature (97)..(98): n is a, c, g, or t
  misc_feature (101)..(101): n is a, c, g, or t
  misc_feature (124)..(124): n is a, c, g, or t
  misc_feature (131)..(131): n is a, c, g, or t
  misc_feature (135)..(137): n is a, c, g, or t
  misc_feature (165)..(165): n is a, c, g, or t
  misc_feature (168)..(168): n is a, c, g, or t
  misc_feature (172)..(172): n is a, c, g, or t
  misc_feature (178)..(178): n is a, c, g, or t
  misc_feature (186)..(186): n is a, c, g, or t
  misc_feature (192)..(192): n is a, c, g, or t
  misc_feature (213)..(213): n is a, c, g, or t
  misc_feature (242)..(242): n is a, c, g, or t
  misc_feature (259)..(259): n is a, c, g, or t
  misc_feature (264)..(264): n is a, c, g, or t
  misc_feature (269)..(269): n is a, c, g, or t
  misc_feature (276)..(276): n is a, c, g, or t
  misc_feature (279)..(279): n is a, c, g, or t
  misc_feature (288)..(288): n is a, c, g, or t
  misc_feature (296)..(296): n is a, c, g, or t
  misc_feature (354)..(354): n is a, c, g, or t
  misc_feature (376)..(376): n is a, c, g, or t
  misc_feature (384)..(384): n is a, c, g, or t
  misc_feature (389)..(389): n is a, c, g, or t
  misc_feature (408)..(409): n is a, c, g, or t
  misc_feature (417)..(422): n is a, c, g, or t
  misc_feature (425)..(426): n is a, c, g, or t
  misc_feature (428)..(428): n is a, c, g, or t
  misc_feature (430)..(431): n is a, c, g, or t
  misc_feature (439)..(439): n is a, c, g, or t
  misc_feature (442)..(442): n is a, c, g, or t
  misc_feature (448)..(448): n is a, c, g, or t
  misc_feature (451)..(451): n is a, c, g, or t
  misc_feature (491)..(491): n is a, c, g, or t
  misc_feature (525)..(525): n is a, c, g, or t
  misc_feature (528)..(528): n is a, c, g, or t
  misc_feature (544)..(544): n is a, c, g, or t
  misc_feature (552)..(552): n is a, c, g, or t
  misc_feature (555)..(555): n is a, c, g, or t
  misc_feature (562)..(563): n is a, c, g, or t
  misc_feature (567)..(567): n is a, c, g, or t
  misc_feature (569)..(569): n is a, c, g, or t
  misc_feature (574)..(574): n is a, c, g, or t
  misc_feature (576)..(576): n is a, c, g, or t
  misc_feature (585)..(585): n is a, c, g, or t
  misc_feature (596)..(596): n is a, c, g, or t
  misc_feature (598)..(598): n is a, c, g, or t
  misc_feature (618)..(618): n is a, c, g, or t
  misc_feature (626)..(626): n is a, c, g, or t
  misc_feature (630)..(630): n is a, c, g, or t
  misc_feature (656)..(656): n is a, c, g, or t
  misc_feature (677)..(677): n is a, c, g, or t
  misc_feature (695)..(695): n is a, c, g, or t
  misc_feature (713)..(713): n is a, c, g, or t
  misc_feature (715)..(715): n is a, c, g, or t
  misc_feature (743)..(743): n is a, c, g, or t
  misc_feature (747)..(747): n is a, c, g, or t
  misc_feature (760)..(760): n is a, c, g, or t
  misc_feature (763)..(767): n is a, c, g, or t
  misc_feature (770)..(770): n is a, c, g, or t
  misc_feature (773)..(773): n is a, c, g, or t
  misc_feature (783)..(783): n is a, c, g, or t
  misc_feature (785)..(785): n is a, c, g, or t
  misc_feature (788)..(788): n is a, c, g, or t
  misc_feature (795)..(795): n is a, c, g, or t
  misc_feature (803)..(803): n is a, c, g, or t
  misc_feature (813)..(813): n is a, c, g, or t
  misc_feature (816)..(816): n is a, c, g, or t
  misc_feature (831)..(832): n is a, c, g, or t
  misc_feature (845)..(846): n is a, c, g, or t
  misc_feature (865)..(865): n is a, c, g, or t
  misc_feature (874)..(874): n is a, c, g, or t
  misc_feature (896)..(896): n is a, c, g, or t
  misc_feature (906)..(906): n is a, c, g, or t
  misc_feature (943)..(944): n is a, c, g, or t
  misc_feature (950)..(950): n is a, c, g, or t
  misc_feature (956)..(956): n is a, c, g, or t
  misc_feature (962)..(962): n is a, c, g, or t
  misc_feature (967)..(967): n is a, c, g, or t
  misc_feature (980)..(980): n is a, c, g, or t
  misc_feature (987)..(987): n is a, c, g, or t
  misc_feature (1007)..(1007): n is a, c, g, or t
  misc_feature (1013)..(1013): n is a, c, g, or t
  misc_feature (1017)..(1017): n is a, c, g, or t
  misc_feature (1020)..(1020): n is a, c, g, or t
  misc_feature (1028)..(1028): n is a, c, g, or t
  misc_feature (1036)..(1036): n is a, c, g, or t
  misc_feature (1039)..(1039): n is a, c, g, or t
  misc_feature (1044)..(1045): n is a, c, g, or t
  misc_feature (1057)..(1057): n is a, c, g, or t
  misc_feature (1066)..(1066): n is a, c, g, or t
  misc_feature (1072)..(1072): n is a, c, g, or t
  misc_feature (1075)..(1075): n is a, c, g, or t
  misc_feature (1081)..(1082): n is a, c, g, or t
  misc_feature (1085)..(1085): n is a, c, g, or t
  misc_feature (1088)..(1088): n is a, c, g, or t
  misc_feature (1098)..(1098): n is a, c, g, or t
  misc_feature (1130)..(1130): n is a, c, g, or t
  misc_feature (1154)..(1154): n is a, c, g, or t
  misc_feature (1214)..(1214): n is a, c, g, or t
  misc_feature (1216)..(1217): n is a, c, g, or t
  misc_feature (1230)..(1230): n is a, c, g, or t
  misc_feature (1232)..(1232): n is a, c, g, or t
  misc_feature (1235)..(1235): n is a, c, g, or t
  misc_feature (1247)..(1247): n is a, c, g, or t
  misc_feature (1278)..(1278): n is a, c, g, or t
  misc_feature (1299)..(1299): n is a, c, g, or t
  misc_feature (1314)..(1314): n is a, c, g, or t
  misc_feature (1324)..(1324): n is a, c, g, or t
  misc_feature (1348)..(1348): n is a, c, g, or t
  misc_feature (1350)..(1350): n is a, c, g, or t
  misc_feature (1353)..(1353): n is a, c, g, or t
  misc_feature (1357)..(1357): n is a, c, g, or t
  misc_feature (1406)..(1407): n is a, c, g, or t
  misc_feature (1422)..(1422): n is a, c, g, or t
  misc_feature (1437)..(1437): n is a, c, g, or t
  misc_feature (1484)..(1484): n is a, c, g, or t
  misc_feature (1488)..(1488): n is a, c, g, or t
  misc_feature (1495)..(1495): n is a, c, g, or t
  misc_feature (1497)..(1497): n is a, c, g, or t
  misc_feature (1508)..(1508): n is a, c, g, or t
  misc_feature (1521)..(1521): n is a, c, g, or t
  misc_feature (1563)..(1563): n is a, c, g, or t
  misc_feature (1575)..(1575): n is a, c, g, or t
  misc_feature (1582)..(1582): n is a, c, g, or t
  misc_feature (1590)..(1591): n is a, c, g, or t
  misc_feature (1606)..(1606): n is a, c, g, or t
  misc_feature (1608)..(1608): n is a, c, g, or t
  misc_feature (1621)..(1621): n is a, c, g, or t
  misc_feature (1624)..(1624): n is a, c, g, or t
  misc_feature (1642)..(1642): n is a, c, g, or t
  misc_feature (1650)..(1650): n is a, c, g, or t
  misc_feature (1653)..(1653): n is a, c, g, or t
  misc_feature (1669)..(1669): n is a, c, g, or t
  misc_feature (1671)..(1671): n is a, c, g, or t
  misc_feature (1679)..(1679): n is a, c, g, or t
  misc_feature (1681)..(1681): n is a, c, g, or t
  misc_feature (1704)..(1704): n is a, c, g, or t
  misc_feature (1712)..(1712): n is a, c, g, or t
  misc_feature (1731)..(1731): n is a, c, g, or t
  misc_feature (1749)..(1749): n is a, c, g, or t
  misc_feature (1752)..(1752): n is a, c, g, or t
  misc_feature (1790)..(1792): n is a, c, g, or t
  misc_feature (1798)..(1798): n is a, c, g, or t
  misc_feature (1832)..(1833): n is a, c, g, or t
  misc_feature (1839)..(1840): n is a, c, g, or t
  misc_feature (1851)..(1851): n is a, c, g, or t
  misc_feature (1859)..(1859): n is a, c, g, or t
  misc_feature (1864)..(1864): n is a, c, g, or t
  misc_feature (1887)..(1887): n is a, c, g, or t
  misc_feature (1891)..(1891): n is a, c, g, or t
  misc_feature (1910)..(1910): n is a, c, g, or t
  misc_feature (1922)..(1922): n is a, c, g, or t
  misc_feature (1935)..(1935): n is a, c, g, or t
  misc_feature (1941)..(1941): n is a, c, g, or t
  misc_feature (1946)..(1946): n is a, c, g, or t
  misc_feature (1949)..(1949): n is a, c, g, or t
  misc_feature (1963)..(1963): n is a, c, g, or t
  misc_feature (1976)..(1976): n is a, c, g, or t
  misc_feature (1983)..(1984): n is a, c, g, or t
  misc_feature (1986)..(1986): n is a, c, g, or t
  misc_feature (1993)..(1993): n is a, c, g, or t
  misc_feature (1999)..(1999): n is a, c, g, or t
  misc_feature (2026)..(2027): n is a, c, g, or t
  misc_feature (2039)..(2041): n is a, c, g, or t
  misc_feature (2051)..(2052): n is a, c, g, or t
  misc_feature (2078)..(2078): n is a, c, g, or t
  misc_feature (2083)..(2083): n is a, c, g, or t
  misc_feature (2112)..(2112): n is a, c, g, or t
  misc_feature (2127)..(2127): n is a, c, g, or t
  misc_feature (2139)..(2139): n is a, c, g, or t
  misc_feature (2154)..(2154): n is a, c, g, or t
  misc_feature (2163)..(2163): n is a, c, g, or t
  misc_feature (2178)..(2178): n is a, c, g, or t
  misc_feature (2184)..(2186): n is a, c, g, or t
  misc_feature (2195)..(2196): n is a, c, g, or t
  misc_feature (2198)..(2198): n is a, c, g, or t
  misc_feature (2200)..(2200): n is a, c, g, or t
  misc_feature (2203)..(2203): n is a, c, g, or t
  misc_feature (2206)..(2206): n is a, c, g, or t
  misc_feature (2208)..(2208): n is a, c, g, or t
  misc_feature (2210)..(2210): n is a, c, g, or t
  misc_feature (2218)..(2218): n is a, c, g, or t
  misc_feature (2230)..(2230): n is a, c, g, or t
  misc_feature (2240)..(2240): n is a, c, g, or t
  misc_feature (2247)..(2247): n is a, c, g, or t
  misc_feature (2252)..(2252): n is a, c, g, or t
  misc_feature (2265)..(2265): n is a, c, g, or t
  misc_feature (2320)..(2320): n is a, c, g, or t
  misc_feature (2332)..(2333): n is a, c, g, or t
  misc_feature (2338)..(2338): n is a, c, g, or t
  misc_feature (2342)..(2342): n is a, c, g, or t
  misc_feature (2353)..(2356): n is a, c, g, or t
  misc_feature (2366)..(2367): n is a, c, g, or t
  misc_feature (2371)..(2372): n is a, c, g, or t
  misc_feature (2385)..(2385): n is a, c, g, or t
  misc_feature (2431)..(2431): n is a, c, g, or t
  misc_feature (2477)..(2477): n is a, c, g, or t
  misc_feature (2483)..(2483): n is a, c, g, or t
  misc_feature (2490)..(2490): n is a, c, g, or t
  misc_feature (2492)..(2492): n is a, c, g, or t
  misc_feature (2494)..(2494): n is a, c, g, or t
ncgtnncgntgcntctctcaagnttagatttntttnnncgtaaanagaggaggancnattgctttaaancccaccaattagctccttcactctcagnnctnaacaatggcttcgactctgggcngcgatgngagnnncgagatagtgtttttcgatcttgagacngcngttncgaccnaatcgggncagccntttgcgattttggagtttggngctatcttagtttgccctatgaagctagnggagctctatagttacnccacnttggntcgaccnacngatctttcnctcatctncacgctcacgaagcgacgaagcggcattacgcgcgacggagttctctctgcacctacnttctctgaaatcgctgatgaantctacganattcnccacggtaagggtttctcnntttttttnnnnnnctnncncnntctctctnacncgaagntanaagtattgattttggtgtttctgtaggacgaatttgggcnggacataacataaagagattcgattgtgtaagantangagatgcatttgcagnaattggtntcnctccccnngagncnaaagntncaattgatncactttcgttntngtctcagaagtttgggaagngagctggngacntgaaggtctctcttttttcgtcttcncgatgataaatctcaaagccnatagcttccttgttatcnttatagatatgaatttcnangtaacttcaaagattcatcactcatcanagtngctaaaatttacnctnnnnnaanaangtagatggcntngcntgctacntatttcgngctaggagancangctcacaggtaaaannagtaaacgatacnntgtgccttttaacgattcnccagttgtntcaatatgggactaaacatggntatgattcancaggagcttagatgatgtccggatgaatcttgaagtnntcaagnactgtncaaccntcttntttctggtattgntgtcttntcatttcttgaataatgatnaactcntaanttnaaaaggantagattanagnggttnngacatatctganttctgtctncagttntgnaaaagnnggntcnatcttccttncagaccacaactttgcaagccgtaaacatggnttgcaacttgcaagtatagtttgntatatcactgagtttaagtacttggtgtttgcaggagtccagtgttcctgacattcttananncatgagctggttntnccnaagaaaaagtcngagaacacgaagtaatgagaagtcactgccnaatggagtcagagaaagcccnacttcttcctcttcnagccctaaanctgatccgagttcgtcttctgtanangcnacanctgtcaaaaaccatcccatcatttctcttctgacggaatgctcagaaanngatacatctagttgngaaatagatccatcngacataaccactctaataagtaaactacatattggaactcttaaganagangctgcgnangaagccaaaantgtaagacagcanggtgaatcaaccgatcccaatgccaaagatgaatcatttttnggcgttaatgangtatctntttctagnntcagggcaagtcttntnccgttatatcgtnggngtctgagaatggagctgnttcacaanganacccctctacatctcngntggtatancntgaaaattcggtttggaataagncggaagtntgtggatcatgtaggtcgnccaaagatgaatattgtngtngacatacctcctgatttatgcaagatcttggacgcatnnnatgctnctgcgcataacttactgattgactcaagcacaanntcagannggaggcctacngttatgangaaanaaggctttgccaactatcccacngccngactgcagtaagtattcancactctctctgnccttttacatacnagcatnaatcnacnggagagtctctaanaccatctccaacnctactcnntnttcaccnccaaantctattttggagttaaatccctccaanncttgcaaaatannnatcttcaaannttttctccatatttggagattttganttttnaagtcatgactccattttggagttgggtnggagaaaaacacaantccaaaataganttacttcattttggngtaaaaaantgaagaaatgggttngagatnnnctaacctcnntnancantcntntnttgttggnagaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatgatctagccaaggaaaaatcattcctctgtctcttnctggtnagtcggngngnactt

SEQ ID NO: 35 - DNA, 1491 bp, Boechera
  misc_feature (19)..(19): n is a, c, g, or t
  misc_feature (26)..(26): n is a, c, g, or t
  misc_feature (30)..(32): n is a, c, g, or t
  misc_feature (60)..(60): n is a, c, g, or t
  misc_feature (63)..(63): n is a, c, g, or t
  misc_feature (67)..(67): n is a, c, g, or t
  misc_feature (73)..(73): n is a, c, g, or t
  misc_feature (81)..(81): n is a, c, g, or t
  misc_feature (87)..(87): n is a, c, g, or t
  misc_feature (108)..(108): n is a, c, g, or t
  misc_feature (137)..(137): n is a, c, g, or t
  misc_feature (154)..(154): n is a, c, g, or t
  misc_feature (159)..(159): n is a, c, g, or t
  misc_feature (164)..(164): n is a, c, g, or t
  misc_feature (171)..(171): n is a, c, g, or t
  misc_feature (174)..(174): n is a, c, g, or t
  misc_feature (183)..(183): n is a, c, g, or t
  misc_feature (191)..(191): n is a, c, g, or t
  misc_feature (249)..(249): n is a, c, g, or t
  misc_feature (271)..(271): n is a, c, g, or t
  misc_feature (279)..(279): n is a, c, g, or t
  misc_feature (284)..(284): n is a, c, g, or t
  misc_feature (303)..(303): n is a, c, g, or t
  misc_feature (337)..(337): n is a, c, g, or t
  misc_feature (340)..(340): n is a, c, g, or t
  misc_feature (356)..(356): n is a, c, g, or t
  misc_feature (364)..(364): n is a, c, g, or t
  misc_feature (367)..(367): n is a, c, g, or t
  misc_feature (374)..(375): n is a, c, g, or t
  misc_feature (379)..(379): n is a, c, g, or t
  misc_feature (381)..(381): n is a, c, g, or t
  misc_feature (386)..(386): n is a, c, g, or t
  misc_feature (388)..(388): n is a, c, g, or t
  misc_feature (397)..(397): n is a, c, g, or t
  misc_feature (408)..(408): n is a, c, g, or t
  misc_feature (410)..(410): n is a, c, g, or t
  misc_feature (430)..(430): n is a, c, g, or t
  misc_feature (438)..(438): n is a, c, g, or t
  misc_feature (442)..(442): n is a, c, g, or t
  misc_feature (453)..(453): n is a, c, g, or t
  misc_feature (455)..(455): n is a, c, g, or t
  misc_feature (458)..(458): n is a, c, g, or t
  misc_feature (465)..(465): n is a, c, g, or t
  misc_feature (473)..(473): n is a, c, g, or t
  misc_feature (483)..(483): n is a, c, g, or t
  misc_feature (486)..(486): n is a, c, g, or t
  misc_feature (528)..(529): n is a, c, g, or t
  misc_feature (535)..(535): n is a, c, g, or t
  misc_feature (541)..(541): n is a, c, g, or t
  misc_feature (547)..(547): n is a, c, g, or t
  misc_feature (552)..(552): n is a, c, g, or t
  misc_feature (584)..(584): n is a, c, g, or t
  misc_feature (586)..(587): n is a, c, g, or t
  misc_feature (600)..(600): n is a, c, g, or t
  misc_feature (602)..(602): n is a, c, g, or t
  misc_feature (605)..(605): n is a, c, g, or t
  misc_feature (617)..(617): n is a, c, g, or t
  misc_feature (648)..(648): n is a, c, g, or t
  misc_feature (669)..(669): n is a, c, g, or t
  misc_feature (684)..(684): n is a, c, g, or t
  misc_feature (694)..(694): n is a, c, g, or t
  misc_feature (718)..(718): n is a, c, g, or t
  misc_feature (720)..(720): n is a, c, g, or t
  misc_feature (723)..(723): n is a, c, g, or t
  misc_feature (727)..(727): n is a, c, g, or t
  misc_feature (776)..(777): n is a, c, g, or t
  misc_feature (792)..(792): n is a, c, g, or t
  misc_feature (807)..(807): n is a, c, g, or t
  misc_feature (854)..(854): n is a, c, g, or t
  misc_feature (858)..(858): n is a, c, g, or t
  misc_feature (865)..(865): n is a, c, g, or t
  misc_feature (867)..(867): n is a, c, g, or t
  misc_feature (878)..(878): n is a, c, g, or t
  misc_feature (891)..(891): n is a, c, g, or t
  misc_feature (933)..(933): n is a, c, g, or t
  misc_feature (945)..(945): n is a, c, g, or t
  misc_feature (952)..(952): n is a, c, g, or t
  misc_feature (960)..(961): n is a, c, g, or t
  misc_feature (976)..(976): n is a, c, g, or t
  misc_feature (978)..(978): n is a, c, g, or t
  misc_feature (991)..(991): n is a, c, g, or t
  misc_feature (994)..(994): n is a, c, g, or t
  misc_feature (1012)..(1012): n is a, c, g, or t
  misc_feature (1020)..(1020): n is a, c, g, or t
  misc_feature (1023)..(1023): n is a, c, g, or t
  misc_feature (1039)..(1039): n is a, c, g, or t
  misc_feature (1041)..(1041): n is a, c, g, or t
  misc_feature (1049)..(1049): n is a, c, g, or t
  misc_feature (1051)..(1051): n is a, c, g, or t
  misc_feature (1074)..(1074): n is a, c, g, or t
  misc_feature (1082)..(1082): n is a, c, g, or t
  misc_feature (1101)..(1101): n is a, c, g, or t
  misc_feature (1119)..(1119): n is a, c, g, or t
  misc_feature (1122)..(1122): n is a, c, g, or t
  misc_feature (1160)..(1162): n is a, c, g, or t
  misc_feature (1168)..(1168): n is a, c, g, or t
  misc_feature (1202)..(1203): n is a, c, g, or t
  misc_feature (1209)..(1210): n is a, c, g, or t
  misc_feature (1221)..(1221): n is a, c, g, or t
  misc_feature (1229)..(1229): n is a, c, g, or t
  misc_feature (1234)..(1234): n is a, c, g, or t
  misc_feature (1257)..(1257): n is a, c, g, or t
  misc_feature (1261)..(1261): n is a, c, g, or t
  misc_feature (1278)..(1278): n is a, c, g, or t
  misc_feature (1288)..(1288): n is a, c, g, or t
  misc_feature (1295)..(1295): n is a, c, g, or t
  misc_feature (1300)..(1300): n is a, c, g, or t
  misc_feature (1313)..(1313): n is a, c, g, or t
  misc_feature (1368)..(1368): n is a, c, g, or t
  misc_feature (1380)..(1381): n is a, c, g, or t
  misc_feature (1386)..(1386): n is a, c, g, or t
  misc_feature (1390)..(1390): n is a, c, g, or t
  misc_feature (1401)..(1404): n is a, c, g, or t
  misc_feature (1414)..(1415): n is a, c, g, or t
  misc_feature (1419)..(1420): n is a, c, g, or t
  misc_feature (1433)..(1433): n is a, c, g, or t
  misc_feature (1479)..(1479): n is a, c, g, or t
atggcttcgactctgggcngcgatgngagnnncgagatagtgtttttcgatcttgagacngcngttncgaccnaatcgggncagccntttgcgattttggagtttggngctatcttagtttgccctatgaagctagnggagctctatagttacnccacnttggntcgaccnacngatctttcnctcatctncacgctcacgaagcgacgaagcggcattacgcgcgacggagttctctctgcacctacnttctctgaaatcgctgatgaantctacganattcnccacggacgaatttgggcnggacataacataaagagattcgattgtgtaagantangagatgcatttgcagnaattggtntcnctccccnngagncnaaagntncaattgatncactttcgttntngtctcagaagtttgggaagngagctggngacntgaagatggcntngcntgctacntatttcgngctaggagancangctcacaggagcttagatgatgtccggatgaatcttgaagtnntcaagnactgtncaaccntcttntttctggagtccagtgttcctgacattcttananncatgagctggttntnccnaagaaaaagtcngagaacacgaagtaatgagaagtcactgccnaatggagtcagagaaagcccnacttcttcctcttcnagccctaaanctgatccgagttcgtcttctgtanangcnacanctgtcaaaaaccatcccatcatttctcttctgacggaatgctcagaaanngatacatctagttgngaaatagatccatcngacataaccactctaataagtaaactacatattggaactcttaaganagangctgcgnangaagccaaaantgtaagacagcanggtgaatcaaccgatcccaatgccaaagatgaatcatttttnggcgttaatgangtatctntttctagnntcagggcaagtcttntnccgttatatcgtnggngtctgagaatggagctgnttcacaanganacccctctacatctcngntggtatancntgaaaattcggtttggaataagncggaagtntgtggatcatgtaggtcgnccaaagatgaatattgtngtngacatacctcctgatttatgcaagatcttggacgcatnnnatgctnctgcgcataacttactgattgactcaagcacaanntcagannggaggcctacngttatgangaaanaaggctttgccaactatcccacngccngactgcaaataagctcngaatccaatngaacccnggtanaccaaaaagaagnacctttgggaaccaatcaaaagctcgatttcagtagcgataattttgaaaagctngagtcagcactnnttccnggtnccctggttgannnnttcttctcanncganncttacgattatangaaaatggtagggatacgtctagcagccagaaagttggtaatccanctgaagaaatga

SEQ ID NO: 36 - DNA, 495 bp, Boechera
  misc_feature (21)..(21): n is a, c, g, or t
  misc_feature (24)..(24): n is a, c, g, or t
  misc_feature (28)..(28): n is a, c, g, or t
  misc_feature (34)..(34): n is a, c, g, or t
  misc_feature (42)..(42): n is a, c, g, or t
  misc_feature (48)..(48): n is a, c, g, or t
  misc_feature (69)..(69): n is a, c, g, or t
  misc_feature (98)..(98): n is a, c, g, or t
  misc_feature (115)..(115): n is a, c, g, or t
  misc_feature (120)..(120): n is a, c, g, or t
  misc_feature (125)..(125): n is a, c, g, or t
  misc_feature (132)..(132): n is a, c, g, or t
  misc_feature (135)..(135): n is a, c, g, or t
  misc_feature (144)..(144): n is a, c, g, or t
  misc_feature (152)..(152): n is a, c, g, or t
  misc_feature (210)..(210): n is a, c, g, or t
  misc_feature (232)..(232): n is a, c, g, or t
  misc_feature (240)..(240): n is a, c, g, or t
  misc_feature (245)..(245): n is a, c, g, or t
  misc_feature (264)..(264): n is a, c, g, or t
  misc_feature (298)..(298): n is a, c, g, or t
  misc_feature (301)..(301): n is a, c, g, or t
  misc_feature (317)..(317): n is a, c, g, or t
  misc_feature (325)..(325): n is a, c, g, or t
  misc_feature (328)..(328): n is a, c, g, or t
  misc_feature (335)..(336): n is a, c, g, or t
  misc_feature (340)..(340): n is a, c, g, or t
  misc_feature (342)..(342): n is a, c, g, or t
  misc_feature (347)..(347): n is a, c, g, or t
  misc_feature (349)..(349): n is a, c, g, or t
  misc_feature (358)..(358): n is a, c, g, or t
  misc_feature (369)..(369): n is a, c, g, or t
  misc_feature (371)..(371): n is a, c, g, or t
  misc_feature (391)..(391): n is a, c, g, or t
  misc_feature (399)..(399): n is a, c, g, or t
  misc_feature (403)..(403): n is a, c, g, or t
  misc_feature (414)..(414): n is a, c, g, or t
  misc_feature (416)..(416): n is a, c, g, or t
  misc_feature (419)..(419): n is a, c, g, or t
  misc_feature (426)..(426): n is a, c, g, or t
  misc_feature (434)..(434): n is a, c, g, or t
  misc_feature (444)..(444): n is a, c, g, or t
  misc_feature (447)..(447): n is a, c, g, or t
  misc_feature (489)..(490): n is a, c, g, or t
gtgtttttcgatcttgagacngcngttncgaccnaatcgggncagccntttgcgattttggagtttggngctatcttagtttgccctatgaagctagnggagctctatagttacnccacnttggntcgaccnacngatctttcnctcatctncacgctcacgaagcgacgaagcggcattacgcgcgacggagttctctctgcacctacnttctctgaaatcgctgatgaantctacganattcnccacggacgaatttgggcnggacataacataaagagattcgattgtgtaagantangagatgcatttgcagnaattggtntcnctccccnngagncnaaagntncaattgatncactttcgttntngtctcagaagtttgggaagngagctggngacntgaagatggcntngcntgctacntatttcgngctaggagancangctcacaggagcttagatgatgtccggatgaatcttgaagtnntcaag

SEQ ID NO: 37 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctcacgcgaagctacaagtattgattttggtgtttctgtaggacgaatttgggtgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcatgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagacatcttcaaaattttctccatatttggagattttgattttttaagtcatgactccattttggagttgggttggagaaaaacacaactccaaaatagatttacttcattttggagtaaaaaatgaagaaatgggttggagatactaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 38 - DNA, 1491 bp, Boechera
atggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggacgaatttgggtgggacataacataaagagattcgattgtgtaagaataagagatgcatttgcagaaattggtctccctcccccggagccgaaagctacaattgattcactttcgttgttgtctcagaagtttgggaagagagctggtgacatgaagatggcatcgcatgctacatatttcgggctaggagatcaagctcacaggagcttagatgatgtccggatgaatcttgaagttatcaagcactgttcaaccgtcttgtttctggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcaaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatga

SEQ ID NO: 39 - DNA, 495 bp, Boechera
gtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggacgaatttgggtgggacataacataaagagattcgattgtgtaagaataagagatgcatttgcagaaattggtctccctcccccggagccgaaagctacaattgattcactttcgttgttgtctcagaagtttgggaagagagctggtgacatgaagatggcatcgcatgctacatatttcgggctaggagatcaagctcacaggagcttagatgatgtccggatgaatcttgaagttatcaag

SEQ ID NO: 40 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaatttaaaaggattagattaaagaggttgagacatatctgacttctgtctacagtttgcaaaagttgggtccatcttccttccagaccacagctttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctcaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgtattcttctcagtcgagccttacgattataagaaaatggtagggatacgtctagcagccagaaagttggtaatccagctgaagaaatgatctagccaaggaaaaatcattcctctgtctcttcctgttcagtcggtgagcactt

SEQ ID NO: 41 - DNA, 1491 bp, Boechera
atggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagttatcaagcactgttcaaccgtcttgtttctggagtccagtgttcctgacattcttacagacatgagctggttattcccaagaaaaagtccgagaacacgaagtaatgagaagtcactgcctaatggagtcagagaaagcccgacttcttcctcttcgagccctcaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcaaataagctcagaatccaatggaacccaggtataccaaaaagaagaacctttgggaaccaatcaaaagctcgatttcagtagcgataattttgaaaagcttgagtcagcactacttcctggtaccctggttgatgtattcttctcagtcgagccttacgattataagaaaatggtagggatacgtctagcagccagaaagttggtaatccagctgaagaaatga

SEQ ID NO: 42 - DNA, 495 bp, Boechera
gtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagttatcaag

SEQ ID NO: 43 - DNA, 2488 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaacagtaaacgataccctgtgccttttaacgattcaccagttgtttcaatatgggactaaacatggatatgattcaccaggagcttagatgatgtccggatgaatcttgaagttatcaagcactgtgcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctttcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcacccgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 44 - DNA, 1491 bp, Boechera
atggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagttatcaagcactgtgcaaccgtcttgtttctggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcaaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcacccgaatcttacgattataagaaaatggtagggatacgtctagcagccagaaagttggtaatccacctgaagaaatga

SEQ ID NO: 45 - DNA, 495 bp, Boechera; identical to SEQ ID NO: 42

SEQ ID NO: 46 - DNA, 2519 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgagagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttctttctcaatctctctgacacgaagctacaagtattgattttggtgtttctgtaggacgaatttgggcgggacataacataaagagattcgattgtgtaagaataagagatgcatttgcaggaattggtgtctctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaagctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtgagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagatatcttcaaaattttctccatatttggagattttgaatttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcactacttcctggtaccctggttgatgcattcttctcactcgagccttacgattataagaaaatggtagggatacgtctagcagccagaaagttggtaatccacctgaagaaatgatctagccaaggaaaaatcattcctctgtctcttgctggtcagtcggtgagcactt

SEQ ID NO: 47 - DNA, 1521 bp, Boechera
atggcttcgactctgggcggcgatgagagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcaggaattggtgtctctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagtagtcaagtactgtgcaaccgtcttgtttctggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtgagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcaaataagctcagaatccaatggaacccaggtacaccaaaaagaagaacctttgggaaccaatcaaaagctcgatttcagtagcgataattttgaaaagcttgagtcagcactacttcctggtaccctggttgatgcattcttctcactcgagccttacgattataagaaaatggtagggatacgtctagcagccagaaagttggtaatccacctgaagaaatga

SEQ ID NO: 48 - DNA, 495 bp, Boechera
gtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcaggaattggtgtctctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagtagtcaag

SEQ ID NO: 49 - DNA, 2487 bp, Boechera
tcgtaccgttgcttctctcaaggttagattttttttccgtaaaaagaggaggatcgattgctttaaaacccaccaattagctccttcactctcagtccttaacaatggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccaccttggttcgacccacagatctttctctcatctccacgctcacgaagcgacgaagcggcattacgcgcgacggagttctctctgcacctacattctctgaaatcgctgatgaagtctacgacattctccacggtaagggtttctcttttttttttctctccatctctctcacacgaaggtacaagtattgattttggtgtttctgtaggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttttttcgtcttctcgatgataaatctcaaagccaatagcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcaccaggagcttagatgatgtccggatgaatcttgaagtagtcaagtactgtgcaaccgtcttatttctggtattgctgtcttctcatttcttgaataatgatcaactctaacttaaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaaaaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctgtcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 50 - DNA, 1491 bp, Boechera
atggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccaccttggttcgacccacagatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagtagtcaagtactgtgcaaccgtcttatttctggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcaaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgaatcttacgattataagaaaatggtagggatacgtctagcagccagaaagttggtaatccacctgaagaaatga

SEQ ID NO: 51 - DNA, 495 bp, Boechera
gtgtttttcgatcttgagacggcagttccgaccaaatcggggcagccttttgcgattttggagtttggggctatcttagtttgccctatgaagctagtggagctctatagttactccaccttggttcgacccacagatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaagatggcatcgcttgctacatatttcgggctaggagatcaagctcacaggagcttagatgatgtccggatgaatcttgaagtagtcaag

SEQ ID NO: 52 - DNA, 2486 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttggttccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctccgttggtatagcttgaaaattcggtttggaataagccggaagtatgtggatcatgtaggtcgtccaaagatgaatattgtcgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcagcactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagatttacttcattttggagtaaaaaatgaagaaatgggttggagatactaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 53 - DNA, 1491 bp, Boechera
atggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaagatggcatcgcttgctacatatttcgggctaggagatcaggctcacaggagcttagatgatgtccggatgaatcttgaagtagtcaagtactgtgcaaccgtcttgtttctggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctccgttggtatagcttgaaaattcggtttggaataagccggaagtatgtggatcatgtaggtcgtccaaagatgaatattgtcgtagacatacctcctgatttatgcaagatcttggacgcatccgatgctgctgcgcataacttactgattgactcaagcacaagctcagattggaggcctactgttatgaggaaagaaggctttgccaactatcccacagccagactgcaaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatga

SEQ ID NO: 54 - DNA, 495 bp, Boechera
gtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaagatggcatcgcttgctacatatttcgggctaggagatcaggctcacaggagcttagatgatgtccggatgaatcttgaagtagtcaag

SEQ ID NO: 55 - DNA, 115 bp, Boechera
  misc_feature (107)..(107): n is a, c, g, or t
tcgtaccgttgcttctctcaagtttagatttttttccgtaaaaagaggaggtggcccgtgaagtttattccctttaaaacccaccaattagctccttcactctcagntctcaaca

SEQ ID NO: 56 - DNA, 105 bp, Boechera
  misc_feature (1)..(1): n is a, c, g, or t
  misc_feature (5)..(6): n is a, c, g, or t
  misc_feature (9)..(9): n is a, c, g, or t
  misc_feature (13)..(13): n is a, c, g, or t
  misc_feature (23)..(23): n is a, c, g, or t
  misc_feature (32)..(32): n is a, c, g, or t
  misc_feature (36)..(38): n is a, c, g, or t
  misc_feature (45)..(45): n is a, c, g, or t
  misc_feature (55)..(55): n is a, c, g, or t
  misc_feature (57)..(57): n is a, c, g, or t
  misc_feature (69)..(69): n is a, c, g, or t
  misc_feature (97)..(98): n is a, c, g, or t
  misc_feature (101)..(101): n is a, c, g, or t
ncgtnncgntgcntctctcaagnttagatttntttnnncgtaaanagaggaggancnattgctttaaancccaccaattagctccttcactctcagnnctnaaca

SEQ ID NO: 57 - DNA, 115 bp, Boechera
tcgtaccgttgcttctctcaagtttagatttttttccgtaaaaagaggaggtggcccgtgaagtttattccctttaaaacccaccaattagctccttcactctcagttctcaaca

SEQ ID NO: 58 - DNA, 115 bp, Boechera; identical to SEQ ID NO: 57

SEQ ID NO: 59 - DNA, 115 bp, Boechera; identical to SEQ ID NO: 57

SEQ ID NO: 60 - DNA, 104 bp, Boechera
tcgtaccgttgcttctctcaagtttagattttttttccgtaaatagaggaggatcaattgctttaaaacccaccaattagctccttcactctcagttctcaaca

SEQ ID NO: 61 - DNA, 104 bp, Boechera
tcgtaccgttgcttctctcaaggttagattttttttccgtaaaaagaggaggatcgattgctttaaaacccaccaattagctccttcactctcagtccttaaca

SEQ ID NO: 62 - DNA, 104 bp, Boechera; identical to SEQ ID NO: 60

SEQ ID NO: 63 - protein, 10 aa, Boechera
DAADEAKTVR

SEQ ID NO: 64 - DNA, 30 bp, Boechera
agatgctgcggacgaagccaaaactgtgag

SEQ ID NO: 65 - DNA, 20 bp, Boechera
tggcccgtgaagtttattcc

SEQ ID NO: 66 - DNA, 10 bp, Boechera
  misc_feature (1)..(3): n is a, c, g, or t
  misc_feature (9)..(10): n is a, c, g, or t
nnnttattnn

SEQ ID NO: 67 - DNA, 10 bp, Boechera
  misc_feature (10)..(10): n is a, c, g, or t
agtttattcn

SEQ ID NO: 68 - DNA, 10 bp, Boechera
  misc_feature (1)..(3): n is a, c, g, or t
  misc_feature (9)..(10): n is a, c, g, or t
nnnggtggnn

SEQ ID NO: 69 - DNA, 12 bp, Boechera
  misc_feature (1)..(7): n is a, c, g, or t
nnnnnnnggtgg

SEQ ID NO: 70 - DNA, 12 bp, Boechera
aagaggaggtgg

SEQ ID NO: 71 - DNA, 10 bp, Boechera
  misc_feature (1)..(3): n is a, c, g, or t
  misc_feature (9)..(10): n is a, c, g, or t
nnnccaccnn

SEQ ID NO: 72 - DNA, 12 bp, Boechera
  misc_feature (6)..(12): n is a, c, g, or t
ccaccnnnnnnn

SEQ ID NO: 73 - DNA, 12 bp, Boechera
ccacctcctctt

SEQ ID NO: 74 - DNA, 10 bp, Boechera
  misc_feature (1)..(3): n is a, c, g, or t
  misc_feature (9)..(10): n is a, c, g, or t
nnngtggcnn

SEQ ID NO: 75 - DNA, 10 bp, Boechera
  misc_feature (1)..(2): n is a, c, g, or t
  misc_feature (8)..(10): n is a, c, g, or t
nngccacnnn

SEQ ID NO: 76 - DNA, 10 bp, Boechera
  misc_feature (1)..(3): n is a, c, g, or t
  misc_feature (9)..(10): n is a, c, g, or t
nnnggcccnn

SEQ ID NO: 77 - DNA, 10 bp, Boechera
  misc_feature (1)..(2): n is a, c, g, or t
  misc_feature (8)..(10): n is a, c, g, or t
nngggccnnn

SEQ ID NO: 78 - DNA, 10 bp, Boechera
  misc_feature (1)..(2): n is a, c, g, or t
  misc_feature (9)..(10): n is a, c, g, or t
nntttattnn

SEQ ID NO: 79 - DNA, 10 bp, Boechera
  misc_feature (1)..(2): n is a, c, g, or t
  misc_feature (9)..(10): n is a, c, g, or t
nnaataaann

SEQ ID NO: 80 - DNA, 11 bp, Boechera
  misc_feature (11)..(11): n is a, c, g, or t
ttgctttaaan

SEQ ID NO: 81 - DNA, 11 bp, Boechera
  misc_feature (1)..(1): n is a, c, g, or t
ntttaaagcaa

SEQ ID NO: 82 - DNA, 10 bp, Boechera
  misc_feature (1)..(2): n is a, c, g, or t
  misc_feature (9)..(10): n is a, c, g, or t
nntgctttnn

SEQ ID NO: 83 - DNA, 10 bp, Boechera
  misc_feature (1)..(2): n is a, c, g, or t
  misc_feature (9)..(10): n is a, c, g, or t
nnaaagcann

SEQ ID NO: 84 - DNA, 10 bp, Boechera
  misc_feature (1)..(3): n is a, c, g, or t
  misc_feature (9)..(10): n is a, c, g, or t
nnngctttnn

SEQ ID NO: 85 - DNA, 10 bp, Boechera
  misc_feature (1)..(2): n is a, c, g, or t
  misc_feature (8)..(10): n is a, c, g, or t
nnaaagcnnn

SEQ ID NO: 86 - DNA, 2490 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 87 - DNA, 2491 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaaagtcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 88 - DNA, 2491 bp, Boechera
tcgtaccgttgcttctctcaagtttagatttttttttccgtaaatagaggaggatcaattgctttaaagcccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctatttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 89 - DNA, 2489 bp, Boechera
tcgtaccgttgcttctctcaagtttagattttttttccgtaaatagaggaggatcaattgctttaaagcccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctatttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 90 - DNA, 2487 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttggttccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagatttacttcattttggagtaaaaaatgaagaaatgggttggagatactaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 91 - DNA, 2488 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttggttccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagatttacttcattttggagtaaaaaatgaagaaatgggttggagatactaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 92 - DNA, 2521 bp, Boechera
tcgtaccgttgcttctctcaagtttagatttttttttccgtaaatagaggaggatcaattgctttaaaacccaccaattagctccttcactctcagttctcaacaatggcttcgactctgggcggcgatgagagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttctttctcaatctctctgacacgaagctacaagtattgattttggtgtttctgtaggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaagctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactcttaacttaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtgagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagatatcttcaaaattttctccatatttggagattttgaatttttaagtcatgactccattttggagttgggttggagaaaaacacaactccaaaatagagttacttcattttggagtaaaaaatgaagaaatgggttggagatactctaacctcagtcaccattcttatgttgttggcagaataagctcagaatccaatggaacccaggtacaccaaaaagaagaacctttgggaaccaatcaaaagctcgatttcagtagcgataattttgaaaagcttgagtcagcactatttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggttagtcggtgagcactt

SEQ ID NO: 93 - DNA, 2490 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaactctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagtatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggtagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 94 - DNA, 2487 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgcttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtatccaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactcattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 95 - DNA, 2489 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgatattctccacggtaagggtttctctcttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagtatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcattaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 96 - DNA, 2487 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaaccacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 97 - DNA, 2491 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttaccccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttttctttctcaatctctctgacacgaagctataagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagtggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtctgagaacacgaagtaatgagaagtcactgcctaatggagtcagagaaagcccaacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcattttttggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccaatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 98 - DNA, 2488 bp, Boechera
tcgtaccgttgcttctctcaaggttagattttttttccgtaaaaagaggaggatcgattgctttaaaacccaccaattagctccttcactctcagttcttaacaatggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccactttggttcgacccacagatctttctctcatctccacgctcacgaagcgacgaagcggcattacgcgcgacggagttctctctgcacctacattctctgaaatcgctgatgaagtctacgacattctccacggtaagggtttctctttttttttttctctccatctctctcacacgaaggtacaagtattgattttggtgtttctgtaggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttttttcgtcttctcgatgataaatctcaaagccaatagcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcaccaggagcttagatgatgtccggatgaatcttgaagtagtcaagtactgtgcaaccgtcttatttctggtattgctgtcttctcatttcttgaataatgatcaactctaacttaaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaaaaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctgtcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 99 - DNA, 2488 bp, Boechera; identical to SEQ ID NO: 98

SEQ ID NO: 100 - DNA, 2488 bp, Boechera
tcgtaccgttgcttctctcaaggttagattttttttccgtaaaaagaggaggatcgattgctttaaaacccaccaattagctccttcactctcagttcttaacaatggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccactttggttcgacccacagatctttctctcatctccacgctcacgaagcgacgaagcggcattacgcgcgacggagttctctctgcacctacattctctgaaatcgctgatgaagtctacgacattctccacggtaagggtttctctttttttttttctctccatctctctcacacgaaggtacaagtattgattttggtgtttctgtaggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttttttcgtcttctcgatgataaatctcaaagccaatagcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcaccaggagcttagatgatgtccggatgaatcttgaagtagtcaagtactgtgcaaccgtcttatttctggtattgctgtcttctcatttcttgaataatgatcaactctaacttaaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatccgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaaaaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctgtcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 101 - DNA, 2475 bp, Boechera
tcgtaccgttgcttctctcaagtttagatttttttgccgtaaatagaggaggatcaattgctttaaaacccaccaattagctccttcactctcagttctcaacaatggcttcgactctgggcggcgatgagagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctgacacgaagctacaagtattgattttggtgtttctgtaggacgaatttgggcaggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttttttcgtcttctcgatgataaatctcaaagcctatagcttgcttgttatctttatagatatgaatttccatgtaacttcaaagattcatcactcatcagagttgctaaaatttactctttttaaaaaatgtagatggcatcgcttgctacatatttcgggctaggagatcaagctcacaggtaaaaagagtaaacgataccatgtgccttttaacgattcaccagttgtttcaatatgggactaaacatgtttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctagtggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataagtaaactacatattggaactcttaagagagatgctgcggacgaagccaaaattgtaagacagcagggtgaatcaaccgatcccaatgccaaagatgaatcatttttgggcgttaatgaagtatctgtttctagcatcagggcaagtcttctcccgttatatcgtgggagtctgagaatggagctgtttcacaatgaaacccctctacatctctgttggtatagcttgaaaattcggtttggaataagccggaagtatgtggatcatgtaggtcgtccaaagatgaatattgttgtagacatacctcctgatttatgcaagatcttggacgcatccgatgcttctgcgcataacttactgattgactcaagcacaagctcagattggaggcctactgttatgaggaaagaaggctttgccaactatcccacagccagactgcagtaagtattcaacactctctctgaccttttacatacgagcatgaatctaccggagagtctctaagaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaatgaagaaatgggttggacatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcactacttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 102 - DNA, 2447 bp, Boechera
tcgtaccgttgcttctctcaagattagatttttttttccgtaaaaagaggaggatcgattgctttaaaacccaccaattagctccttcactctcagttcttaacaatggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccactttggttcgacccacagatctttctctcatctccacgctcacgaagcgacgaagcggcattacgcgcgacggagttctctctgcacctacattctctgaaatcgctgatgaagtctacgacattctccacggtaagggtttctctcttttttttttctccatctctctcacacgaaggtacaagtattgattttggtgtttctgtaggacgaatttgggcgggacataacataaagagattcgattgtgtaagaataagagatgcatttgcaggaattggtctctctcccccggagccgaaagctacaattgattcactttcgttgttgtctcagaagtttgggaagagagctggtgacatgaaggtctctcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcatcaggagcttagatgatgtccggatgaatcttgaagtagtcaagtactgtgcaaccgtcttgtttctggtattgctgtcttctcatttcttgaataatgatcaactctaacttaaaaggattagattagagaggttgagacatatctgacttctgtctacagtttgcaaaagttgggtcaatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctttcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcacccgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 103 - DNA, 2483 bp, Boechera holboellii
tcgtaccgttgcttctctcaaggttagattttttttccgtaaaaagaggaggatcgattgctttaaaacccaccaattagctccttcactctcagttcttaacaatggcttcgactctgggcggcgatgagagatgcgagatagtgtttttcgatcttgagacggcagttccgaccaaatcggggcagccttttgcgattttggagtttggggctatcttagtttgccctatgaagctagtggagctctatagttactccactttggttcgacccacagatctttctctcatctccacgctcacgaagcgacgaagcggcattacgcgcgacggagttctctctgcacctacattctctgaaatcgctgatgaagtctacgacattctccacggtaagggtttctctttttttttttttctctccatctctctcacacgaaggtacaagtattgattttggtgtttctgtaggacgaatttgggcgggacataacataaagagattcgattgtgtacgaataagagatgcatttgcaggaattggtctctctcccccggagccgaaattgattcactttcgttgttgtctcagaagtttgggaagagagctggtgacatgaaggtctctcttttttcgtcttctcgatgataaatctcaaagccaatagcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcaccaggagcttagatgatgtccggatgaatcttgaagtagtcaagtactgtgcaaccgtcttatttctggtattgctgtcttctcatttcttgaataatgatcaactctaacttaaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaaaaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctgtcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgaatcttacgattataagaaaatggtagggatacgtctagcagccagaaagttggtaatccacctgaagaaatgatctagccaaggaaaaatcatttctctgtctcttcctggtcagtcggtgagcactt

SEQ ID NO: 104 - DNA, 2488 bp, Boechera
tcgtaccgttgcttctctcaaggttagattttttttccgtaaaaagaggaggatcgattgctttaaaacccaccaattagctccttcactctcagttcttaacaatggcttcgactctgggctgcgatgagagatgcgagatagtgtttttcgatcttgagacggcagttccgaccaaatcggggcagccttttgcgattttggagtttggggctatcttagtttgccctatgaagctagtggagctctatagttactccactttggttcgacccacagatctttctctcatctccacgctcacgaagcgacgaagcggcattacgcgcgacggagttctctctgcacctacattctctgaaatcgctgatgaagtctacgacattctccacggtaagggtttctctttttttttttctctccatctctctcacacgaaggtacaagtattgattttggtgtttctgtaggacgaatttgggcaggacataacataaagagattcgattgtgtaagaataagagatgcatttgcaggaattggtctctctcccccggagtcaaaagctacaattgattcactttcgttgttgtctcagaagtttgggaagagagctggtgacatgaaggtctctcttttttcgtcttctcgatgataaatctcaaagccaatagcttccttgttatctttatagatatgaatttccatgtaacttcaaagattcatcactcatcagagttgctaaaatttactctttttcaataacgtagatggcatcgcttgctacatatttcgggctaggagatcaagctcacaggtaaaaagagtaaacgataccctgtgccttttaacgattcaccagttgtttcaatatgggactaaacatggttatgattcaccaggagcttagatgatgtccggatgaatcttgaagtagtcaagtactgtgcaaccgtcttatttctggtattgctgtcttctcatttcttgaataatgatcaactctaacttaaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggatgaagccaaaattgtaagacagcagggtgaatcaaccgatcccaatgccaaagatgaatcatttttgggcgttaatgaagtatctgtttctagcatcagggcaagtcttatcccgttatatcgtgggagtctgagaatggagctgcttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatacgatgctgctgcgcataacttactgattgactcaagcacaagctcagattggaggcctactgttatgatgaaagaaggctttgccaactatcccacagccagactgcagtaagtattcaacactctctctgaccttttacatacgagcatgaatccaccggagagtctctaaaaccatctccaaccctactccgtattcacctccaaactctattttggagttaaatccctccaacccttgcaaaatagatatcttcaaaattttctccatatttggagattttgattttttaagtcatgactccattttggagttgggttggagaaaaacacaactccaaaatagagttacttcattttggagtaaaaaaatgaagaaatgggttggagatactctaacctctgtcaccattcttatgttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcgttcttctcactcgaatcttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 105 - DNA, 2518 bp, Boechera
tcgtaccgttgcttctctcaagtttagatttttttccgtaaatagaggaggatcaattgctttaaaacccaccaattagctccttcactctcagttctcaacaatggcttcgactctgggcggcgatgagagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctcttttttttttttctttctcaatctctctgacacgaagctacaagtattgattttggtgtttctgtaggacgaatttgggcgggacataacataaagagattcgattgtgtaagaataagagatgcatttgcaggaattggtgtctctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaagctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgacttctgtctacagtttgcaaaagttgggtccatcttcctttcagaccacaactttgcaagccgtaaacatgggttgcaacttgcaagtatagtttgtcatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtgagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggagtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagatatcttcaaaattttctccatatttggagattttgaatttttaagtcatgactccattttggagttgggttggagaaaaacacaactccaaaatagagttacttcattttggagtaaaaaatgaagaaatgggttggagatactctaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcactacttcctggtaccctggttgatgcattcttctcactcgagccttacgattataagaaaatggtagggatacgtctagcagccagaaagttggtaatccacctgaagaaatgatctagccaaggaaaaatcattcctctgtctcttgctggtcagtcggtgagcactt

SEQ ID NO: 106 - DNA, 2488 bp, Boechera
tcgtaccgttgcttctctcaagtttagattttttttccgtaaatagaggaggatcaattgctttaaaacccaccaattagctccttcactctcagttctcaacaatggcttcgactctgggcggcgatgggagatgcgagatagtgtttttcgatcttgagacggcggttccgaccaaatcggggcagccttttgcgattttggagtttggggctatcttagtttgccctatgaagctagtggagctctatagttactccactttggttcgacccaccgatctttctctcatctccacgctcacgaagcgacgaagcggcattacgcgcgacggagttctctctgcacctacattctctgaaatcgctgatgaagtctacgacattctccacggtaagggtttctctttttttttttttctttctcaatctctctgacacgaagctacaagtattgattttggtgtttctgtaggacgaatttgggcgggacataacataaagagattcgattgtgtaagaatacgagatgcatttgcaggaattggtctctctcccccggagccgaaagctacaattgattcactttcgttattgtctcagaagtttgggaagagagctggtgacatgaaggtctctcttttttcgtcttctcgatgataaatctcaaagcctatagcttccttgttatctttatagatatgaatttccatgtaacttcaaagattcatcactcatcagagttgctaaaatttactctttttaaaaaatgtagatggcatcgcttgctacatatttcgggctaggagatcaggctcacaggtaaaaagagtaaacgataccatgtgccttttaacgattcaccagttgtttcaatatgggactaaacatggttatgattcaccaggagcttagatgatgtccggatgaatcttgaagtagtcaagtactgtgcaaccgtcttgtttctggtattgctgtcttttcatttcttgaataatgattaactctaacttaaaaggattagattagagaggttgagacatatctgatttctgtctacagtttgcaaaagttgggtccatcttcctttcagaccacaactttgcaagccgtaaacatgggttgcaacttgcaagtatagtttgttatatcactgagtttaagtacttggtgtttgcaggagtccagtgttcctgacattcttaaagacatgagctggttttccccaagaaaaagtccgagaacacgaagtaatgagaagtcactgcctaatggagtcagagaaagcccgacttcttcctcttcaagccctaaaactgatccgagttcgtcttctgtagatgccacaactgtcaaaaaccatcccatcatttctcttctgacggaatgctcagaaagtgatacatctagttgtgaaatagatccatctgacataaccactctaataagtaaactacatattggaactcttaagagagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtgggggtctgagaatggagctgtttcacaatgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctactgttatgaggaaagaaggctttgccaactatcccacagccagactgcagtaagtattcaacactctctctgaccttttacatacgagcatgaatccaccggagagtctctaagaccatctccaaccctactccgtattcacctccaaactctattttggagttaaatccctccaacccttgcaaaatagacatcttcaaaattttctccatatttggagattttgattttttaagtcatgactccattttggagttgggttggagaaaaacacaactccaaaatagatttacttcattttggagtaaaaaatgaagaaatgggttggagatactaacctctgtcaccattcttatgttgttggcagaataagctcagaatccaatggaacccaggtacaccaaaaagaagaacctttgggaaccaatcaaaagctcgatttcagtagcgataattttgaaaagcttgagtcagcacttcttcctggtaccctggttgatgcattcttctcactcgagccttacgattataagaaaatggtagggatacgtctagcagccagaaagttggtaatccacctgaagaaatgatctagccaaggaaaaatcattcctctgtctcttcctggtcagtcggtgagcactt

SEQ ID NO: 107 - DNA, 2485 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 108 - DNA, 2487 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgtgcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctatattcacctccaaactctattttggagttaaatccctccaacccttgcaaaatagagatcttcaaattttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttttcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcactcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 109 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacataagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 110 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatggagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 111 - DNA, 2486 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcatgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 112 - DNA, 2484 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcgttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtataacttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 113 - DNA, 2486 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctctttttttttttctttctcaatctctctcacgcgaagctacaagtattgattttggtgtttctgtaggacgaatttgggcaggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcagtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 114 - DNA, 2485 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttctgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcatgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtctagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaagtccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 115 - DNA, 2483 bp, Boechera; identical to SEQ ID NO: 40

SEQ ID NO: 116 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaagactgtaagacagcagggtgaatcaaccgatcccaatgccaaagatgaatcatttttgggcgttaatgaagtatctgtttctaacatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgtattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 117 - DNA, 2486 bp, Boechera; identical to SEQ ID NO: 111

SEQ ID NO: 118 - DNA, 2483 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcttgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagtgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataggccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctattcacctccaaactctattttggagttaaatccctccaacccttgcaaaatagagatcttcaaatttttctccatatttggagattttgatttttaagtcatgactccattttggagttgggttggagaaaaacacaattccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt

SEQ ID NO: 119 - DNA, 2485 bp, Boechera
tcgtaccgttgcttctctcaagtttagatttttttccgtaaaaagaggaggtggcccgtgaagtttattccctttaaaacccaccaattagctccttcactctcagttctcaacaatggcttcgactctgggcggcgatgagagaaacgagatagtgtttttcgatcttgagactgcggttccgaccaaatcggggcagccttttgcgattttggagtttggggctatcttagtttgccctatgaagctagtggagctctatagttactccactttggttcgacctaccgatctttctctcatctccacgctcacgaagcgacgaagcggcattacgcgcgacggagttctctctgcacctacattctctgaaatcgctgatgaagtctacgacattctccacggtaagggtttctcttttttttttctttctcaatctctctcacgcgaagctacaagtattgattttggtgtttctgtaggacgaatttgggcgggacataacataaagagattcgattgtgtaagaataagagatgcatttgcagaaattggtctccctcccccggagccgaaagctacaattgattcactttcgttgttgtctcagaagtttgggaagagagctggtgacatgaaggtctctcttttttcgtcttctcgatgataaatctcaaagcctatagcttccttgttatctttatagatatgaatttcaatgtaacttcaaagattcatcactcatcaaagttgctaaaatttactctaaataatgtagatggcatcgcatgctacatatttcgggctaggagatcaagctcacaggtaaaagagtaaacgataccctgtgccttttaacgattcaccagttgtttcaatatgggactaaacatggatatgattcaccaggagcttagatgatgtccggatgaatcttgaagttatcaagcactgttcaaccgtcttgtttctggtattgttgtcttctcatttcttgaataatgattaactctaacttaaaaggattagattaaagaggttgagacatatctgacttctgtctacagtttgcaaaagttgggtccatcttccttccagaccacaactttgcaagccgtaaacatggtttgcaagtatagtttgtcatatcactgagtttaagtacttggtgtttgcaggagtccagtgttcctgacattcttacagacatgagctggttattcccaagaaaaagtccgagaacacgaagtaatgagaagtcactgcctaatggagtcagagaaagcccgacttcttcctcttcgagccctaaaactgatccgagttcgtcttctgtagatgccacagctgtcaaaaaccatcccatcatttctcttctgacggaatgctcagaaagcgatacatctagttgtgaaatagatccatctgacataaccactctaataagtaaactacatattggaactcttaagacagatgctgcggacgaagccaaaactgtaagacagcagggtgaatcaaccgatcccaatgccaaagatgaatcatttttgggcgttaatgaagtatctgtttctagcatcagggcaagtcttatcccgttatatcgtaggagtctgagaatggagctgtttcacaacgacacccctctacatctctgttggtatagcttgaaaattcggtttggaataagccggaagtatgtggatcatgtaggtcgtccaaagatgaatattgttgtagacatacctcctgatttatgcaagatcttggacgcatccgatgctgctgcgcataacttactgattgactcaagcacaagctcagattggaggcctaccgttatgaggaaaaaaggctttgccaactatcccacagccagactgcagtaagtattcaacactctctctgaccttttacatacgagcatgaatccaccggagagtctctaagaccatctccaaccctactctctattcacctccaaactctattttggagttaaatccctccaacccttgcaaaatagagatcttcaaatttttctccatatttggagattttgatttttaagtcatgactccattttggagttgggttggagaaaaacacaagtccaaaatagagttacttcattttggagtaaaaaatgaagaaatgggttcgagatgctctaacctctgtcaccattcttatcttgttggcagaataagctcagaatccaatggaacccaggtataccaaaaagaagaacctttgggaaccaatcaaaagctcgatttcagtagcgataattttgaaaagcttgagtcagcactacttcctggtaccctggttgatgcattcttctcagtcgagccttacgattataagaaaatggtagggatacgtctagcagccagaaagttggtaatccagctgaagaaatgatctagccaaggaaaaatcattcctctgtctcttcctggtcagtcggtgagcactt